SEQUENCE LISTING (1 sequence)

SEQ ID NO: 1 - protein, 31 aa, Homo sapiens
HAEGTFTSDVSSYLEGQAAKEFIAWLVKGRG

(19) United States
(12) Patent Application Publication
(10) Pub. No.: US 2016/0136246 A1
(43) Pub. Date: May 19, 2016

(54) Novel Uses of GLP-1 Receptor Agonists in Patients Treated with Insulin and/or Suffering from Type 1 Diabetes

(71) Applicant: NOVO NORDISK A/S, Bagsværd (DK)

(72) Inventor: Erik Christiansen, Charlottenlund (DK)

(21) Appl. No.: 14/899,821

(22) PCT Filed: Jun. 23, 2014
(86) PCT No.: PCT/EP2014/063115

§ 371 Date: Dec. 18, 2015

Related U.S. Application Data
(60) Provisional application No. 61/837,988, filed on Jun. 21, 2013.

(30) Foreign Application Priority Data
Jun. 21, 2013 (EP) 13173316.4

(51) Int. Cl. A61K 38/26; A61K 38/28

(52) U.S. Cl. CPC A61K 38/26; A61K 38/28

(57) ABSTRACT

The present invention relates to GLP-1 receptor agonists for use in hypoglycaemia or hypoglycaemic episodes in patients treated with insulin and/or suffering from type 1 diabetes, e.g. for reducing the number of hypoglycaemia or hypoglycaemic episodes.

### FIELD OF THE INVENTION

The present invention relates to GLP-1 receptor agonists for use in hypoglycaemia or hypoglycaemic episodes in patients treated with insulin and/or suffering from type 1 diabetes, e.g. for reducing the number of hypoglycaemia or hypoglycaemic episodes.

### BACKGROUND OF THE INVENTION

Nocturnal hypoglycaemia causes recurrent morbidity in most people with type 1 diabetes and is sometimes fatal. It frequently occurs with insulin therapy, and although blood glucose levels are often low during sleep, they are seldom measured routinely. Almost 50% of all episodes of severe hypoglycaemia occur at night during sleep. Such episodes can cause convulsions and coma and have been implicated as a precipitating factor in cardiac arrhythmias resulting in sudden death.

Hypoglycaemia can result from exogenous or endogenous insulin excess alone. However, nocturnal hypoglycaemia is typically the result of insulin excess and compromised glucose counterregulation. Decreases in insulin, increases in glucagon and/or epinephrine normally prevent or rapidly correct hypoglycaemia.

In insulin-deficient diabetes, exogenous insulin levels do not decrease as glucose levels fall, and the combination of deficient glucagon and epinephrine responses causes defective glucose counterregulation. Reduced sympathoadrenal responses cause hypoglycaemia unawareness. Hypoglycaemia causes both defective glucose counterregulation and hypoglycaemia unawareness, leading to a vicious cycle of recurrent hypoglycaemia and further impairment of glucose counterregulation.

The hormones regulating insulin secretion belong to the so-called enteroinsular axis, designating a group of hormones, released from the gastrointestinal mucosa in response to the presence and absorption of nutrients in the gut, which promote an early and potentiated release of insulin. The enhancing effect on insulin secretion, the so-called incretin effect, is probably essential for a normal glucose tolerance.

GLP-1, a product of the proglucagon, is one of the youngest members of the secretin-VIP family of peptides, but is already established as an important gut hormone with regulatory function in glucose metabolism and gastrointestinal secretion and metabolism. The glucagon gene is processed differently in the pancreas and in the intestine. In the pancreas, the processing leads to the formation and parallel secretion of 1) glucagon itself, occupying positions 33-61 of proglucagon; 2) an N-terminal peptide of 30 amino acids (proglucagon(1-30)) often called glicentin-related pancreatic peptide, GRPP; 3) a hexapeptide corresponding to proglucagon(64-69); 4) and, finally, the so-called major proglucagon fragment (proglucagon(72-158)), in which the two glucagon-like sequences are buried. Glucagon seems to be the only biologically active product. In contrast, in the intestinal mucosa, it is glucagon that is buried in a larger molecule, while the two glucagon-like peptides are formed separately (8). The following products are formed and secreted in parallel: 1) glicentin, corresponding to proglucagon(1-69), with the glucagon sequence occupying residues Nos. 33-61; 2) GLP-1(7-36)amide (proglucagon(78-107))amide, not as originally believed proglucagon(72-107) amide or 108, which is inactive). Small amounts of C-terminally glycine-extended but equally bioactive GLP-1(7-37), (proglucagon(78-108)) are also formed; 3) intervening peptide-2 (proglucagon(111-122)amide) (15); and 4) GLP-2 (proglucagon(126-158)). A fraction of glicentin is cleaved further into GRPP (proglucagon(1-30)) and oxyntomodulin (proglucagon(33-69)). Of these peptides, GLP-1 has the most conspicuous biological activities.

Being secreted in parallel with glicentin/enteroglucagon, it follows that the many studies of enteroglucagon secretion to some extent also apply to GLP-1 secretion, but GLP-1 is metabolised more quickly with a plasma half-life in humans of 2 min. Carbohydrate or fat-rich meals stimulate secretion, presumably as a result of direct interaction of yet unabsorbed nutrients with the microvilli of the open-type L-cells of the gut mucosa. Endocrine or neural mechanisms promoting GLP-1 secretion may exist but have not yet been demonstrated in humans.

The incretin function of GLP-1(29-31) has been clearly illustrated in experiments with the GLP-1 receptor antagonist, exendin 9-39, which dramatically reduces the incretin effect elicited by oral glucose in rats. The hormone interacts directly with the β-cells via the GLP-1 receptor which belongs to the glucagon/VIP/calcitonin family of G-protein-coupled 7-transmembrane spanning receptors. The importance of the GLP-1 receptor in regulating insulin secretion was illustrated in recent experiments in which a targeted disruption of the GLP-1 receptor gene was carried out in mice. Animals homozygous for the disruption had greatly deteriorated glucose tolerance and fasting hyperglycaemia, and even heterozygous animals were glucose intolerant. The signal transduction mechanism primarily involves activation of adenylate cyclase, but elevations of intracellular Ca2+ are also essential. The action of the hormone is best described as a potentiation of glucose stimulated insulin release, but the mechanism that couples glucose and GLP-1 stimulation is not known. It may involve a calcium-induced calcium release. As already mentioned, the insulinotropic action of GLP-1 is preserved in diabetic β-cells. The relation of the latter to its ability to convey “glucose competence” to isolated insulin-secreting cells, which respond poorly to glucose or GLP-1 alone, but fully to a combination of the two, is also not known.

However, the hormone also potently inhibits glucagon secretion. The mechanism is not known, but seems to be paracrine, via neighbouring insulin or somatostatin cells. Also the glucagonostatic action is glucose-dependent, so that the inhibitory effect decreases as blood glucose decreases. Because of this dual effect, if the plasma GLP-1 concentrations increase either by increased secretion or by exogenous infusion the molar ratio of insulin to glucagon in the blood that reaches the liver via the portal circulation is greatly increased, whereby hepatic glucose production decreases. As a result blood glucose concentrations decrease. Because of the glucose dependency of the insulinotropic and glucagonostatic actions, the glucose lowering effect is self-limiting, and the hormone, therefore, does not cause hypoglycaemia regardless of dose. The effects are preserved in patients with diabetes mellitus, in whom infusions of slightly supraphysiological doses of GLP-1 may completely normalise blood glucose values in spite of poor metabolic control and secondary failure to sulphonylurea.

Postprandial hyperglucagonaemia in subjects with type 1 diabetes (IDDM) is known to be a contributing factor to hyperglycaemia and a limiting factor in achieving glycaemic control. Hyperglucagonaemia in type 1 diabetes is proposed to be caused by the near total absence of endogenous insulin secretion from the β-cells and hereby impaired glucagon suppressive effects in the postprandial state. Inhibitors of glucagon secretion, such as glucagon-like peptide-1 (GLP-1) receptor agonists are therefore likely to be able to mitigate this effect in type 1 diabetes.

The glucagonostatic effect of GLP-1 is of interest in regards to the physiological defence mechanisms to hypoglycaemia. Glucagon is an important counterregulatory hormone to hypoglycaemia, but glucagon response is attenuated or absent in type 1 diabetes, and is inversely correlated to the duration of diabetes. Therefore, subjects with type 1 diabetes rely on other counterregulatory hormones than glucagon, such as epinephrine, during a hypoglycaemic event. However, also epinephrine has been shown to be attenuated in long-standing type 1 diabetes resulting in an increased risk of severe hypoglycaemia. Furthermore, reduction of the sympathetic neural responses is also seen during hypoglycaemia leading to impaired awareness of hypoglycaemia and reduced behavioural defence mechanisms (e.g. carbohydrate intake).

Recovery to euglycaemic levels after a hypoglycaemic episode is impaired in subjects with long-standing type 1 diabetes compared to subjects with recent-onset diabetes, likely related to the counterregulatory responses of cortisol and growth hormone. The mechanism of the attenuated response to hypoglycaemia is not known. If this response is further compromised by treatment with a GLP-1 receptor agonist, it could prevent the safe use of GLP-1 receptor agonists in the treatment of type 1 diabetes either alone or as adjuncts to insulin treatment in subjects with type 1 diabetes.

Hence, there is a continuing need to provide interventions for type 1 diabetes, particularly in the reduction of hypoglycaemic episodes associated with insulin therapy.

### SUMMARY

In an alternative or additional embodiment the invention relates to a method for reducing i) the number of hypoglycaemic episodes suffered by; ii) the severity of blood glucose excursions suffered by; iii) the amount of exogenous glucose needed for recovering from a hypoglycaemic episode in; and/or iv) the duration of hypoglycaemic episodes; in a subject being treated with insulin, said method comprising administering to said subject an amount of a GLP-1 receptor agonist effective to reduce i), ii), iii) and/or iv), respectively.

In an alternative or additional embodiment the invention relates to a method for treating type 1 diabetes in a subject in need of such treatment, said method comprising administering to said subject an amount of a GLP-1 receptor agonist effective to treat type 1 diabetes and reduce i) the number of hypoglycaemic episodes suffered by said subject; ii) the severity of blood glucose excursions suffered by said subject; iii) the amount of exogenous glucose needed for recovering from a hypoglycaemic episode for said subject; iv) the duration of hypoglycaemic episodes in said subject.

In an alternative or additional embodiment the invention relates to a GLP-1 receptor agonist for use in treating type 1 diabetes, wherein said use comprises reducing i) the number of hypoglycaemic episodes; ii) the severity of blood glucose excursions; iii) the amount of exogenous glucose needed for recovering from a hypoglycaemic episode; and/or iv) the duration of hypoglycaemic episodes.

### BRIEF DESCRIPTION OF DRAWINGS

FIG. 1 shows the trial design.

FIG. 2 shows total daily insulin dose during treatment.

FIG. 3 shows ADA classification of hypoglycaemia.

FIG. 4 shows change in body weight after 4 weeks of treatment.

FIG. 5 shows hypoglycaemic clamp design.

FIG. 6 shows glucagon concentration during the hypoglycaemic clamp.

FIG. 7 shows concentration of other counterregulatory hormones during the hypoglycaemic clamp.

FIG. 8 shows glucose infusion rate during the hypoglycaemic clamp.

FIG. 9 shows statistical analysis of hypoglycaemic episodes (IG≦3.9 mmol/L) during CGM.

FIG. 10 shows statistical analysis of duration of hypoglycaemic episodes (IG≦3.9 mmol/L) during CGM.

### DETAILED DESCRIPTION OF THE INVENTION

The present invention relates to i.a. reducing the frequency of and/or the symptoms of hypoglycaemias or hypoglycaemic episodes by administration of GLP-1 receptor agonists, for example in patients treated with insulin and/or suffering from type 1 diabetes.

In an alternative or additional embodiment the invention relates to a method for reducing i) the number of hypoglycaemias or hypoglycaemic episodes suffered by; ii) the severity of blood glucose excursions suffered by; iii) the amount of exogenous glucose needed for recovering from a hypoglycaemia in; and/or iv) the duration of hypoglycaemias or hypoglycaemic episodes; in a subject being treated with insulin, said method comprising administering to said subject an amount of a GLP-1 receptor agonist effective to reduce i), ii), iii) and/or iv), respectively.

In an alternative or additional embodiment the invention relates to a method for treating type 1 diabetes in a subject in need of such treatment, said method comprising administering to said subject an amount of a GLP-1 receptor agonist effective to treat type 1 diabetes and reduce i) the number of hypoglycaemias or hypoglycaemic episodes suffered by said subject; ii) the severity of blood glucose excursions suffered by said subject; iii) the amount of exogenous glucose needed for recovering from a hypoglycaemia for said subject; iv) the duration of hypoglycaemias or hypoglycaemic episodes in said subject.

In an alternative or additional embodiment the invention relates to a GLP-1 receptor agonist for use in treating type 1 diabetes, wherein said use comprises reducing i) the number of hypoglycaemias or hypoglycaemic episodes; ii) the severity of blood glucose excursions; iii) the amount of exogenous glucose needed for recovering from a hypoglycaemia; and/or iv) the duration of hypoglycaemias or hypoglycaemic episodes.

In an alternative or additional embodiment the invention relates to the use of a GLP-1 agonist in the preparation of a medicament for i) reducing the number of hypoglycaemic episodes; ii) reducing the severity of blood glucose excursions; iii) reducing the amount of exogenous glucose needed for recovering from a hypoglycaemic episode; iv) reducing the duration of hypoglycaemic episodes; and/or v) treating or preventing hypoglycaemia or hypoglycaemic episodes; in patients suffering from type 1 diabetes.

A first alternative or additional embodiment of the invention provides a GLP-1 receptor agonist for use in treating or preventing hypoglycaemia or hypoglycaemic episodes in patients suffering from type 1 diabetes. A second alternative or additional embodiment of the invention provides the use of a GLP-1 agonist in the preparation of a medicament for treating or preventing hypoglycaemia or hypoglycaemic episodes in patients suffering from type 1 diabetes. A third alternative or additional embodiment of the invention provides a method of treating or preventing hypoglycaemia or hypoglycaemic episodes in patients suffering from type 1 diabetes comprising administering an effective amount of a GLP-1 agonist to an individual in need of treatment. Unless otherwise stated, the following embodiments may be embodiments of the first, second and/or third alternative or additional embodiments of the invention. Similarly, embodiments described herein may be combined, unless otherwise stated.

In an alternative or additional embodiment the reduction as referred to herein is as compared to the subject, to whom the GLP-1 receptor agonist is administered, prior to treatment with a GLP-1 receptor agonist. In an alternative or additional embodiment, treatment or prevention according to the present invention comprises administration of insulin in addition to the GLP-1 receptor agonist of the invention or placebo thereof. In an alternative or additional embodiment the invention relates to treatment of type 1 diabetes, wherein a GLP-1 receptor agonist is administered adjunct to insulin.

In an alternative or additional embodiment, unless otherwise stated, the term “placebo” as used herein (in particular when the placebo is of a GLP-1 receptor agonist) refers to treatment or prevention comprising placebo in respect of a, or the, GLP-1 receptor agonist of the invention, i.e. treatment or prevention using the same composition as that of the GLP-1 receptor agonist and not comprising the GLP-1 receptor agonist itself. Accordingly, “placebo” as used herein may comprise administration of other therapeutically active ingredients other than a GLP-1 receptor agonist which the subject was administered before administration of the GLP-1 receptor agonist. In an alternative or additional embodiment the terms “placebo” and “placebo not undergoing treatment with a, or the, GLP-1 receptor agonist of the invention” (in particular when the placebo is of a GLP-1 receptor agonist) refers to the subject, to whom the GLP-1 receptor agonist is administered, prior to treatment with a GLP-1 receptor agonist of the invention.

### Hypoglycaemia

The terms “hypoglycaemia”, “hypoglycaemic episodes” or “hypoglycaemia episodes associated with insulin therapy” may include hypoglycaemia or hypoglycaemic episodes experienced during insulin therapy of diabetes, such as type 1 diabetes. In an alternative or additional embodiment, by “hypoglycaemia or hypoglycaemic episodes associated with insulin therapy of type 1 diabetes” we include hypoglycaemia or hypoglycaemic episodes experienced during insulin therapy of type 1 diabetes. In an alternative or additional embodiment, we include hypoglycaemia or hypoglycaemic episodes resulting from or exacerbated by insulin therapy (i.e., wherein the hypoglycaemia or hypoglycaemic episodes are absent, less frequent or less pronounced compared to placebo not receiving insulin therapy).

By “hypoglycaemia” we include that this is a physiological state at which the plasma glucose levels are lower than 3.9 mmol/L. Plasma glucose levels may be measured according to any suitable means known in the art, however, In an alternative or additional embodiment plasma glucose levels are determined according to the method described in Konig et al.: Comparative determinations of glucose concentrations in the urine with polarity and enzyme method hexokinaseglucose-6-phosphate dehydrogenase, Schweiz Med Wochenschr 101:860-866, 1971 which is incorporated by reference herein.

By “hypoglycaemic episode” we include that a physical state of hypoglycaemia is present for a certain period of time, which varies from patient to patient depending on their ability to respond/react on the hypoglycaemic state and provide an intervention/treatment. In untreated patients the hypoglycaemic episode may be present a few seconds, minutes, or hours. In an alternative or additional embodiment the hypoglycaemic episode is present for more than 5 seconds, such as more than 10 seconds, more than 20 seconds, or more than 30 seconds. In an alternative or additional embodiment the hypoglycaemic episode is present for more than 1 minute, such as more than 5 minutes, more than 10 minutes, or more than 30 minutes. In an alternative or additional embodiment the hypoglycaemic episode is present for more than 1 hour, such as more than 1.5 hour, more than 2 hours, or more than 3 hours.

Accordingly, in an alternative or additional embodiment hypoglycaemia is defined as a plasma glucose level of 3.9 mmol/L (70 mg/dL) or less.

In an alternative or additional embodiment hypoglycaemia is defined as a plasma glucose level of 3.9 mmol/L or less as well as one or more hypoglycaemic symptoms. As used herein the term “hypoglycaemic symptoms” refers to neuroglycopenic symptoms selected from the group consisting of difficulty thinking, confused, tired, drowsy, weak and/or faint, dizzy and seizure and/or coma; and/or neuropenic symptoms selected from the group consisting of heart pounding, tremulous, sweaty (diaphoresis), hungry, tingling, and pallor (pale appearance). In an alternative or additional embodiment the hypoglycaemic symptoms are neuroglycopenic symptoms. In an alternative or additional embodiment the hypoglycaemic symptoms are neuropenic symptoms.

In an alternative or additional embodiment the hypoglycaemia is severe (also referred to as major). In a severe hypoglycaemia the affected subject is unable to treat him/herself and is in need of assistance from another person to e.g. actively administer carbohydrate, glucagon, or take other corrective actions to restore plasma glucose levels in said subject. Plasma glucose values may not be available during severe hypoglycaemia, but neurological recovery following the return of plasma glucose to normal is considered sufficient evidence that the event was induced by a low plasma glucose concentration. In an alternative or additional embodiment severe hypoglycaemia may further be defined as i) plasma blood glucose level of less than 3.9 mmol/L (70 mg/dL), such as less than 2.2 mmol/L (40 mg/dL) or less than 2.8 mmol/L (50 mg/dL); ii) recovery of the subject from the severe hypoglycaemia following intake of carbohydrate, glucagon, or other corrective actions; or iii) the subject affected by the severe hypoglycaemia is in coma, had seizures, or died. The term “recovery of the subject from the severe hypoglycaemia” may be defined as the affected subject having a plasma glucose level of more than 3.9 mmol/L (70 mg/dL) in said subject. Alternatively, the term “recovery of the subject from the severe hypoglycaemia” may be defined as the affected subject no longer needing assistance from another person to e.g. actively administer carbohydrate, glucagon, or take other corrective actions to restore plasma glucose levels in said subject.

In an alternative or additional embodiment the hypoglycaemia is non-severe (also referred to as minor). In a non-severe hypoglycaemia the affected person can treat the hypoglycaemia him/herself without the need of assistance from another person.

In an alternative or additional embodiment the non-severe hypoglycaemia is defined by a plasma blood glucose level in the range of 2.2-3.8 mmol/L (40-69 mg/dL); also referred to herein as mild-to-moderate hypoglycaemia.

In an alternative or additional embodiment the non-severe hypoglycaemia is asymptomatic hypoglycaemia which is defined by a plasma glucose level of 3.9 mmol/L (70 mg/dL) or less and no hypoglycaemic symptoms.

In an alternative or additional embodiment the non-severe hypoglycaemia is documented symptomatic hypoglycaemia which is defined by a plasma glucose level of 3.9 mmol/L (70 mg/dL) or less and one or more hypoglycaemic symptoms.

In an alternative or additional embodiment the non-severe hypoglycaemia is pseudo-hypoglycaemia which is defined by a plasma glucose level of more than 3.9 mmol/L (70 mg/dL) and one or more hypoglycaemic symptoms.

In an alternative or additional embodiment the non-severe hypoglycaemia is probable symptomatic hypoglycaemia which is defined by no measurement of plasma glucose level and one or more hypoglycaemic symptoms.

In an alternative or additional embodiment, the terms “hypoglycaemia”, “hypoglycaemic episode”, “hypoglycaemic event” and/or “hypo” are used interchangeably herein.

In an alternative or additional embodiment the GLP-1 receptor agonist for use in relation to hypoglycaemia, such as insulin induced hypoglycaemia, reactive hypoglycaemia, diabetic hypoglycaemia, non-diabetic hypoglycaemia, fasting hypoglycaemia, drug-induced hypoglycaemia, gastric by-pass induced hypoglycaemia, hypoglycaemia in pregnancy, alcohol induced hypoglycaemia, and/or insulinoma.

In an alternative or additional embodiment the hypoglycaemia or hypoglycaemic episodes may be selected from the group consisting of: hypoglycaemia; overall hypoglycaemic episodes; nocturnal hypoglycaemic episodes; and daytime hypoglycaemic episodes.

By “overall hypoglycaemic episodes” we include the number of hypoglycaemic episodes experienced over a period of time, for example, over one day, one day and night (i.e. 24 hours), seven days, four weeks, 42 weeks or more. In an alternative or additional embodiment the overall hypoglycaemic episodes is the number of hypoglycaemic episodes experienced over a period of time of seven days, four weeks, or one year. The term “overall hypoglycaemic episodes” includes daytime hypoglycaemic episodes and nocturnal hypoglycaemic episodes.

By “daytime hypoglycaemic episodes” we include as hypoglycaemia experienced when awake. In particular, we include hypoglycaemia experience during day time. In particular, we include hypoglycaemia experience during non-fasting wakefulness (i.e., when the patient has been fasting for less than 6 hours, for example, 5, 4.5, 4, 3.5, 3, 2.5, 2, 1.5, 1, 0.5 or 0 hours). In particular, we include hypoglycaemia experience during fasting wakefulness (i.e., when the patient has been fasting for more than 6 hours, for example, 5, 4.5, 4, 3.5, 3, 2.5, 2, 1.5, 1, 0.5 or 0 hours).

By “nocturnal hypoglycaemia” we include as hypoglycaemia experienced during sleep. In particular, we include hypoglycaemia experience during sleep at night time. In particular, we include hypoglycaemia experience during fasting sleep (i.e., when the patient has been fasting for 6 or more hours, for example, 7, 8, 9 10, 11, 12, 13, 14, 15, 16, 17 or 18 hours, or more). In particular, we include hypoglycaemia experience during non-fasting sleep (i.e., when the patient has been fasting for less than 6 hours, for example, 5, 4.5, 4, 3.5, 3, 2.5, 2, 1.5, 1, 0.5 or 0 hours).

### Number of Hypoglycaemias

In an alternative or additional embodiment the invention relates to use of the GLP-1 receptor agonist for reducing the number of hypoglycaemias. By “reducing the number of hypoglycaemias” we include reducing the number of hypoglycaemias experienced over a period of time. Accordingly, in an alternative or additional embodiment the invention relates to use of the GLP-1 receptor agonist for reducing the number of hypoglycaemias experienced over a period of time, for example, over one day (i.e. 24 hours), seven days, four weeks, 42 weeks, one year or more compared to placebo not undergoing treatment with a, or the, GLP-1 receptor agonist of the invention.

In an alternative or additional embodiment the invention relates to a GLP-1 receptor agonist for use in treating type 1 diabetes, wherein said use comprises reducing the number of hypoglycaemias or hypoglycaemic episodes.

In an alternative or additional embodiment the invention relates to a method for reducing the number of hypoglycaemias or hypoglycaemic episodes suffered by a subject being treated with insulin, said method comprising administering to said subject an amount of a GLP-1 receptor agonist effective to reduce the number of hypoglycaemias or hypoglycaemic episodes suffered by said subject.

In an alternative or additional embodiment the invention relates to a method for treating type 1 diabetes in a subject in need of such treatment, said method comprising administering to said subject an amount of a GLP-1 receptor agonist effective to treat type 1 diabetes and reduce the number of hypoglycaemias or hypoglycaemic episodes suffered by said subject.

In an alternative or additional embodiment the GLP-1 receptor agonist is capable of reducing the number of hypoglycaemias by at least 25%, for example, 30%, 35%, or 40%.

In an alternative or additional embodiment the GLP-1 receptor agonist is capable of reducing the number of hypoglycaemias by at least 31%, when dose of said GLP-1 receptor agonist is in the range of 0.5-2.0 mg/day, such as 0.6 mg/day, 1.2 mg/day or 1.8 mg/day. In an alternative or additional embodiment the GLP-1 receptor agonist is capable of reducing the number of hypoglycaemias by at least 31%, when dose of said GLP-1 receptor agonist is 0.6 mg/day. In an alternative or additional embodiment the GLP-1 receptor agonist is capable of reducing the number of hypoglycaemias by at least 35%, when dose of said GLP-1 receptor agonist is 1.2 mg/day. In an alternative or additional embodiment the GLP-1 receptor agonist is capable of reducing the number of hypoglycaemias by at least 39%, when dose of said GLP-1 receptor agonist is 1.8 mg/day.

In an alternative or additional embodiment the GLP-1 receptor agonist is capable of reducing the number of hypoglycaemias to 1.2 or fewer, such as 1.1 or fewer, events per person days of exposure (PDE). In an alternative or additional embodiment the GLP-1 receptor agonist is capable of reducing the number of hypoglycaemias to 1.0 or fewer, such as 0.9 or fewer or 0.8 or fewer, events per person days of exposure (PDE).

In an alternative or additional embodiment the GLP-1 receptor agonist is capable of reducing the number of hypoglycaemias to 1.2 or fewer events per person days of exposure (PDE) when dose of said GLP-1 receptor agonist is in the range of 0.5-2.0 mg/day, such as 0.6 mg/day, 1.2 mg/day or 1.8 mg/day.

In an alternative or additional embodiment reducing the number of hypoglycaemias experienced over a period of time is reducing overall hypoglycaemic episodes.

By “reducing the number of hypoglycaemias experienced over a period of time” we include reducing the number of hypoglycaemias experienced over a period of time, for example, over one day (i.e. 24 hours), seven days, four weeks, 42 weeks, one year or more, compared to placebo not undergoing treatment with a, or the, GLP-1 receptor agonist of the invention. In an alternative or additional embodiment the invention relates to use of the GLP-1 receptor agonist for reducing overall hypoglycaemic episodes (i.e. the number of hypoglycaemic episodes experienced over a period of time), e.g. over a period of time of 4 weeks, 42 weeks, or one year.

By “reducing overall hypoglycaemic episodes” we include reducing the number of hypoglycaemic episodes experienced over a period of time, for example, over one day (i.e. 24 hours), seven days, four weeks, 42 weeks, one year or more compared to placebo not undergoing treatment with a, or the, GLP-1 receptor agonist of the invention.

In an alternative or additional embodiment the GLP-1 receptor agonist is capable of reducing overall hypoglycaemic episodes by at least 25%, for example, 30%, 35%, or 40%.

In an alternative or additional embodiment the GLP-1 receptor agonist is capable of reducing the number of hypoglycaemias or hypoglycaemic episodes by at least 31%. In an alternative or additional embodiment the GLP-1 receptor agonist is capable of reducing the number of hypoglycaemias or hypoglycaemic episodes by at least 35%. In an alternative or additional embodiment the GLP-1 receptor agonist is capable of reducing the number of hypoglycaemias or hypoglycaemic episodes by at least 39%.

In an alternative or additional embodiment the GLP-1 receptor agonist is capable of reducing overall hypoglycaemic episodes by at least 31%, when dose of said GLP-1 receptor agonist is in the range of 0.5-2.0 mg/day, such as 0.6 mg/day, 1.2 mg/day or 1.8 mg/day. In an alternative or additional embodiment the GLP-1 receptor agonist is capable of reducing overall hypoglycaemic episodes by at least 31%, when dose of said GLP-1 receptor agonist is 0.6 mg/day. In an alternative or additional embodiment the GLP-1 receptor agonist is capable of reducing overall hypoglycaemic episodes by at least 35% %, when dose of said GLP-1 receptor agonist is 1.2 mg/day. In an alternative or additional embodiment the GLP-1 receptor agonist is capable of reducing overall hypoglycaemic episodes by at least 39%, when dose of said GLP-1 receptor agonist is 1.8 mg/day.

In an alternative or additional embodiment the GLP-1 receptor agonist is capable of reducing overall hypoglycaemic episodes to 1.2 or fewer, such as 1.1 or fewer, events per person days of exposure (PDE). In an alternative or additional embodiment the GLP-1 receptor agonist is capable of reducing overall hypoglycaemic episodes to 1.0 or fewer, such as 0.9 or fewer or 0.8 or fewer, events per person days of exposure (PDE).

In an alternative or additional embodiment the GLP-1 receptor agonist is capable of reducing overall hypoglycaemic episodes to 1.2 or fewer events per person days of exposure (PDE) when dose of said GLP-1 receptor agonist is in the range of 0.5-2.0 mg/day, such as 0.6 mg/day, 1.2 mg/day or 1.8 mg/day.

### Severity of Blood Glucose Excursions

In an alternative or additional embodiment the invention relates to reducing the severity of blood glucose excursions. Accordingly, in an alternative or additional embodiment the GLP-1 receptor agonist is capable of reducing the severity of blood glucose excursions. By “reducing the severity of blood glucose excursions” we include reducing the degree to which blood plasma levels fall below 3.9 mmol/L during blood glucose excursions compared to placebo not undergoing treatment with a, or the, GLP-1 receptor agonist of the invention.

In an alternative or additional embodiment the invention relates to a GLP-1 receptor agonist for use in treating type 1 diabetes, wherein said use comprises reducing the severity of blood glucose excursions.

In an alternative or additional embodiment the invention relates to a method for reducing the severity of blood glucose excursions suffered by a subject being treated with insulin, said method comprising administering to said subject an amount of a GLP-1 receptor agonist effective to reduce the severity of blood glucose excursions suffered by said subject.

In an alternative or additional embodiment the invention relates to a method for treating type 1 diabetes in a subject in need of such treatment, said method comprising administering to said subject an amount of a GLP-1 receptor agonist effective to treat type 1 diabetes and reduce the severity of blood glucose excursions suffered by said subject.

In an alternative or additional embodiment the GLP-1 receptor agonist, when administered at a dose 1.2 mg once daily, is capable of reducing the amount of exogenous glucose needed for recovering after hypoglycaemia or a hypoglycaemic episode by at least 19%.

In an alternative or additional embodiment the GLP-1 receptor agonist, when administered in a dose 0.6 mg once daily, is capable of reducing the amount of exogenous glucose needed for recovering after hypoglycaemia or a hypoglycaemic episode by at least 23%.

In an alternative or additional embodiment the GLP-1 receptor agonist, when administered in a dose 1.8 mg once daily, is capable of reducing the amount of exogenous glucose needed for recovering after hypoglycaemia or a hypoglycaemic episode by at least about 37% relative to placebo not undergoing treatment with a, or the, GLP-1 receptor agonist of the invention.

By “reducing the severity of blood glucose excursions” we include reducing the degree to which blood plasma levels fall below 3.9 mmol/L (i.e., hypoglycaemia) and/or rise above 7.8 mmol/L (i.e., hyperglycaemia) during blood glucose excursions compared to placebo not undergoing treatment with a, or the, GLP-1 receptor agonist of the invention. In an alternative or additional embodiment the severity of blood glucose excursions is reduced by at least 5%. In an alternative or additional embodiment the severity of blood glucose excursions is reduced by at least 10%. In an alternative or additional embodiment the severity of blood glucose excursions is reduced by at least 15%. In an alternative or additional embodiment the severity of blood glucose excursions is reduced by at least 20%. In an alternative or additional embodiment the severity of blood glucose excursions is reduced by at least 25%. In an alternative or additional embodiment the severity of blood glucose excursions is reduced by at least 30%. In an alternative or additional embodiment the severity of blood glucose excursions is reduced by at least 40%. In an alternative or additional embodiment the severity of blood glucose excursions is reduced by at least 50%. In an alternative or additional embodiment the severity of blood glucose excursions is reduced by at least 60%. In an alternative or additional embodiment the severity of blood glucose excursions is reduced by at least 70%. In an alternative or additional embodiment the severity of blood glucose excursions is reduced by at least 80%. In an alternative or additional embodiment the severity of blood glucose excursions is reduced by at least 90%. In an alternative or additional embodiment the severity of blood glucose excursions is reduced by at least 95%. In an alternative or additional embodiment the severity of blood glucose excursions is reduced by at least 97.5%. In an alternative or additional embodiment the severity of blood glucose excursions is reduced by at least 99%. In an alternative or additional embodiment the severity of blood glucose excursions is reduced by at least 100%.

### Exogenous Glucose

In an alternative or additional embodiment the invention relates to reducing the amount of exogenous glucose needed for recovering from a hypoglycaemia.

In an alternative or additional embodiment the GLP-1 receptor agonist is capable of reducing the amount of exogenous glucose needed for recovering from a hypoglycaemic episode and/or reducing the severity of blood glucose excursions.

In an alternative or additional embodiment the invention relates to a GLP-1 receptor agonist for use in treating type 1 diabetes, wherein said use comprises reducing the amount of exogenous glucose needed for recovering from a hypoglycaemia or a hypoglycaemic episode.

In an alternative or additional embodiment the invention relates to a method for reducing the amount of exogenous glucose needed for recovering from a hypoglycaemia or a hypoglycaemic episode in a subject being treated with insulin, said method comprising administering to said subject an amount of a GLP-1 receptor agonist effective to reduce the amount of exogenous glucose needed for recovering from a hypoglycaemia or a hypoglycaemic episode by said subject.

In an alternative or additional embodiment the invention relates to a method for treating type 1 diabetes in a subject in need of such treatment, said method comprising administering to said subject an amount of a GLP-1 receptor agonist effective to treat type 1 diabetes and reduce the amount of exogenous glucose needed for recovering from a hypoglycaemia or a hypoglycaemic episode suffered by said subject.

By “reducing the amount of exogenous glucose needed for recovering from a hypoglycaemia” we include reducing the units of exogenous glucose required to restore plasma glucose from less than 3.9 mmol/L to 3.9 mmol/L or greater compared to placebo not undergoing treatment with a, or the, GLP-1 receptor agonist of the invention. In an alternative or additional embodiment the amount of exogenous glucose needed for recovering from a hypoglycaemia is reduced by at least 5%, such as at least 10%, at least 15%, or at least 20%. In an alternative or additional embodiment the amount of exogenous glucose needed for recovering from a hypoglycaemia is reduced by at least 25%, such as at least 30%, at least 40%, or at least 50%. In an alternative or additional embodiment the amount of exogenous glucose needed for recovering from a hypoglycaemia is reduced by at least 60%, such as at least 70%, at least 80%, or at least 90%. In an alternative or additional embodiment the amount of exogenous glucose needed for recovering from a hypoglycaemia is reduced by at least 95%, such as at least 97.5%, at least 99%, or 100%.

By “reducing the amount of exogenous glucose needed for recovering from a hypoglycaemic episode” we include reducing the units of exogenous glucose required to restore plasma glucose from less than 3.9 mmol/L to 3.9 mmol/L or greater compared to placebo not undergoing treatment with a, or the, GLP-1 receptor agonist of the invention. In an alternative or additional embodiment the amount of exogenous glucose needed for recovering from a hypoglycaemic episode is reduced by at least 5%. In an alternative or additional embodiment the amount of exogenous glucose needed for recovering from a hypoglycaemic episode is reduced by at least 10%. In an alternative or additional embodiment the amount of exogenous glucose needed for recovering from a hypoglycaemic episode is reduced by at least 15%. In an alternative or additional embodiment the amount of exogenous glucose needed for recovering from a hypoglycaemic episode is reduced by at least 20%. In an alternative or additional embodiment the amount of exogenous glucose needed for recovering from a hypoglycaemic episode is reduced by at least 25%. In an alternative or additional embodiment the amount of exogenous glucose needed for recovering from a hypoglycaemic episode is reduced by at least 30%. In an alternative or additional embodiment the amount of exogenous glucose needed for recovering from a hypoglycaemic episode is reduced by at least 40%. In an alternative or additional embodiment the amount of exogenous glucose needed for recovering from a hypoglycaemic episode is reduced by at least 50%. In an alternative or additional embodiment the amount of exogenous glucose needed for recovering from a hypoglycaemic episode is reduced by at least 60%. In an alternative or additional embodiment the amount of exogenous glucose needed for recovering from a hypoglycaemic episode is reduced by at least 70%. In an alternative or additional embodiment the amount of exogenous glucose needed for recovering from a hypoglycaemic episode is reduced by at least 80%. In an alternative or additional embodiment the amount of exogenous glucose needed for recovering from a hypoglycaemic episode is reduced by at least 90%. In an alternative or additional embodiment the amount of exogenous glucose needed for recovering from a hypoglycaemic episode is reduced by at least 95%. In an alternative or additional embodiment the amount of exogenous glucose needed for recovering from a hypoglycaemic episode is reduced by at least 97.5%. In an alternative or additional embodiment the amount of exogenous glucose needed for recovering from a hypoglycaemic episode is reduced by at least 99%. In an alternative or additional embodiment the amount of exogenous glucose needed for recovering from a hypoglycaemic episode is reduced by at least 100%.

In an alternative or additional embodiment the GLP-1 receptor agonist is administered at a dose sufficient to reduce the exogenous glucose requirement of the patient to recover from hypoglycaemia or a hypoglycaemic episode.

In an alternative or additional embodiment the GLP-1 receptor agonist is administered at a dose sufficient to maximally reduce the exogenous glucose requirement in the patient to recover from a potential hypoglycaemia or potential hypoglycaemic episode.

In an alternative or additional embodiment the GLP-1 receptor agonist administered is liraglutide and the dose sufficient to optimally reduce the exogenous glucose requirement in the patient recovering from potential hypoglycaemias is between 0.6 mg per day and 1.8 mg per day (for example, between 0.6 mg and 1.2 mg per day).

In an alternative or additional embodiment the GLP-1 receptor agonist administered is liraglutide and the dose is sufficient to optimally reduce the exogenous glucose requirement in the patient recovering from potential hypoglycaemic episodes is between 0.6 mg per day and 1.8 mg per day (for example, between 0.6 mg and 1.2 mg per day).

In an alternative or additional embodiment the GLP-1 receptor agonist is liraglutide and the dose sufficient to optimally reduce the exogenous glucose requirement in the patient recovering from potential hypoglycaemias is selected from the group consisting of 0.6 mg per day; 1.2 mg per day and 1.8 mg per day.

In an alternative or additional embodiment the GLP-1 receptor agonist is liraglutide and the dose sufficient to optimally reduce the exogenous glucose requirement in the patient recovering from potential hypoglycaemic episodes is selected from the group consisting of 0.6 mg per day; 1.2 mg per day and 1.8 mg per day.

In an alternative or additional embodiment the GLP-1 receptor agonist is administered at a dose of 0.6 mg once daily to reduce the exogenous glucose requirement of the patient to recover from hypoglycaemia or a hypoglycaemic episode.

In an alternative or additional embodiment the GLP-1 receptor agonist is administered at a dose of 1.2 mg once daily to reduce the exogenous glucose requirement of the patient to recover from hypoglycaemia or a hypoglycaemic episode.

In an alternative or additional embodiment the GLP-1 receptor agonist is administered at a dose of 1.8 mg once daily to reduce the exogenous glucose requirement of the patient to recover from hypoglycaemia or a hypoglycaemic episode.

In an alternative or additional embodiment the GLP-1 receptor agonist, when administered at a dose 1.2 mg once daily, is capable of reducing the amount of exogenous glucose needed for recovering after a hypoglycaemic episode by at least 19%. In an alternative or additional embodiment the GLP-1 receptor agonist, when administered in a dose 0.6 mg once daily, is capable of reducing the amount of exogenous glucose needed for recovering after a hypoglycaemic episode by at least 23%. In an alternative or additional embodiment the GLP-1 receptor agonist, when administered in a dose 1.8 mg once daily, is capable of reducing the amount of exogenous glucose needed for recovering after a hypoglycaemic episode by at least about 37% relative to placebo.

In an alternative or additional embodiment the GLP-1 receptor agonist is administered at a dose sufficient to reduce the exogenous glucose requirement of the patient to recover from a hypoglycaemic episode. In an alternative or additional embodiment the GLP-1 receptor agonist is administered at a dose sufficient to maximally reduce the exogenous glucose requirement in the patient to recover from a potential hypoglycaemic episode.

In an alternative or additional embodiment the GLP-1 receptor agonist is administered at a dose of 0.6 mg once daily to reduce the exogenous glucose requirement of the patient to recover from a hypoglycaemic episode. In an alternative or additional embodiment the GLP-1 receptor agonist is administered at a dose of 1.2 mg once daily to reduce the exogenous glucose requirement of the patient to recover from a hypoglycaemic episode. In an alternative or additional embodiment the GLP-1 receptor agonist is administered at a dose of 1.8 mg once daily to reduce the exogenous glucose requirement of the patient to recover from a hypoglycaemic episode.

### Duration of Hypoglycaemic Episodes

In an alternative or additional embodiment the invention relates to reducing the duration of hypoglycaemias. Accordingly, in an alternative or additional embodiment the GLP-1 receptor agonist is capable of reducing the duration of hypoglycaemia or hypoglycaemic episodes. By “reducing the duration of hypoglycaemias” we include reducing the duration of hypoglycaemias experienced compared to placebo not undergoing treatment with a, or the, GLP-1 receptor agonist of the invention over a period of time, e.g. over a period of time of 24 hours or more, such as 7 days, 4 weeks, or one year. By “reducing the duration of hypoglycaemic episodes” we include reducing the duration of hypoglycaemic episodes experienced compared to placebo not undergoing treatment with a, or the, GLP-1 receptor agonist of the invention over a period of time, e.g. over a period of time of 24 hours or more, such as 7 days, 4 weeks, or one year.

In an alternative or additional embodiment the invention relates to a GLP-1 receptor agonist for use in treating type 1 diabetes, wherein said use comprises reducing the duration of hypoglycaemia or hypoglycaemic episodes.

In an alternative or additional embodiment the invention relates to a method for reducing the duration of hypoglycaemias or hypoglycaemic episodes in a subject being treated with insulin, said method comprising administering to said subject an amount of a GLP-1 receptor agonist effective to reduce the duration of hypoglycaemias or hypoglycaemic episodes suffered by said subject.

In an alternative or additional embodiment the invention relates to a method for treating type 1 diabetes in a subject in need of such treatment, said method comprising administering to said subject an amount of a GLP-1 receptor agonist effective to treat type 1 diabetes and reduce the duration of hypoglycaemias or hypoglycaemic episodes suffered by said subject.

In an alternative or additional embodiment the GLP-1 receptor agonist is capable of reducing the duration of hypoglycaemias by at least 5%, such as at least 10%, at least 15% or at least 20%. In an alternative or additional embodiment the GLP-1 receptor agonist is capable of reducing the duration of hypoglycaemias by at least 30%, such as at least 40%, at least 45% or at least 50%.

In an alternative or additional embodiment the GLP-1 receptor agonist is capable of reducing the duration of hypoglycaemias by at least 20%, wherein said GLP-1 receptor agonist is administered in a dose of at least 1.0 mg per day, such as 1.0-3.0 mg per day.

In an alternative or additional embodiment the GLP-1 receptor agonist is capable of reducing the duration of hypoglycaemias by at least 0.1 hour per 24 hours, such as at least 0.2 hours per 24 hours, at least 0.3 hours per 24 hours or at least 0.4 hours per 24 hours. In an alternative or additional embodiment the GLP-1 receptor agonist is capable of reducing the duration of hypoglycaemias by at least 0.5 hours per 24 hours, such as at least 0.6 hours per 24 hours, at least 0.7 hours per 24 hours or at least 0.8 hours per 24 hours.

In an alternative or additional embodiment the GLP-1 receptor agonist is capable of reducing the duration of hypoglycaemias by at least 0.5 hours per 24 hours, wherein said GLP-1 receptor agonist is administered in a dose of at least 1.0 mg per day, such as 1.0-3.0 mg per day.

### Blood or Plasma Glucose Measurement

Blood or plasma glucose measurements may be taken using any suitable method known in the art. However, preferably, blood/plasma glucose blood samples are assayed using a hexokinase-UV method. More preferably, blood/plasma glucose are performed using a lancet and glucose monitor for self-measurement of blood glucose (SMBG) glucose by the patient. For more information on SMBG see Benjamin, 2002, ‘Self-Monitoring of Blood Glucose: The Basics’ Clinical Diabetes; 20(1):45-47 which is incorporated by reference herein.

Preferably the sample provided is a blood sample (preferably a capillary blood sample) but equally, the blood sample provided may be a plasma sample. Preferably the fasting blood/plasma glucose measurement is taken at the same time on each day of measurement, or within ±5 hours of that time. In an alternative or additional embodiment the fasting blood/plasma glucose measurement is taken within ±4.5 hours of that time. In an alternative or additional embodiment the fasting blood/plasma glucose measurement is taken within ±4 hours of that time. In an alternative or additional embodiment the fasting blood/plasma glucose measurement is taken within ±3.5 hours of that time. In an alternative or additional embodiment the fasting blood/plasma glucose measurement is taken within ±3 hours of that time. In an alternative or additional embodiment the fasting blood/plasma glucose measurement is taken within ±2.5 hours of that time. In an alternative or additional embodiment the fasting blood/plasma glucose measurement is taken, within ±2 hours of that time. In an alternative or additional embodiment the fasting blood/plasma glucose measurement is taken within ±1.5 hours of that time. In an alternative or additional embodiment the fasting blood/plasma glucose measurement is taken within ±1 hour of that time. In an alternative or additional embodiment the fasting blood/plasma glucose measurement is taken within, ±45 min of that time. In an alternative or additional embodiment the fasting blood/plasma glucose measurement is taken within ±30 min of that time. In an alternative or additional embodiment the fasting blood/plasma glucose measurement is taken within ±15 min of that time. In an alternative or additional embodiment the fasting blood/plasma glucose measurement is taken within ±10 min of that time. In an alternative or additional embodiment the fasting blood/plasma glucose measurement is taken within ±5 min of that time. In an alternative or additional embodiment the fasting blood/plasma glucose measurement is taken within or ±1 min of that time). Preferably, the fasting blood/glucose measurement is taken at least 8 hours after eating (most preferably at least 9, 10, 11 or 12 hours after eating). Preferably, the blood/plasma glucose measurement is taken before breakfast.

The blood/glucose measurement may be a fasting or a non-fasting blood/plasma glucose measurement.

Two major methods have been used to measure glucose. The first, still in use in some places, is a chemical method exploiting the nonspecific reducing property of glucose in a reaction with an indicator substance that changes color when reduced. Since other blood compounds also have reducing properties (e.g., urea, which can be abnormally high in uremic patients), this technique can produce erroneous readings in some situations (5 to 15 mg/dL has been reported). The more recent technique, using enzymes specific to glucose, is less susceptible to this kind of error. The two most common employed enzymes are glucose oxidase and hexokinase.

In either case, the chemical system is commonly contained on a test strip which is inserted into a meter, and then has a blood sample applied. Test-strip shapes and their exact chemical composition vary between meter systems and cannot be interchanged. Formerly, some test strips were read (after timing and wiping away the blood sample) by visual comparison against a color chart printed on the vial label. Strips of this type are still used for urine glucose readings, but for blood glucose levels they are obsolete. Their error rates were, in any case, much higher.

Urine glucose readings, however taken, are much less useful. In properly functioning kidneys, glucose does not appear in urine until the renal threshold for glucose has been exceeded. This is substantially above any normal glucose level, and is evidence of an existing severe hyperglycaemic condition. However, as urine is stored in the bladder, any glucose in it might have been produced at any time since the last time the bladder was emptied. Since metabolic conditions change rapidly, as a result of any of several factors, this is delayed news and gives no warning of a developing condition. Blood glucose monitoring is far preferable, both clinically and for home monitoring by patients. Healthy urine glucose levels were first standardized and published in 1965 by Hans Renschler.

Treating and/or Preventing Hypoglycaemia

In an alternative or additional embodiment the invention relates to a GLP-1 receptor agonist for use in treating or preventing hypoglycaemia or hypoglycaemic episodes in patients suffering from type 1 diabetes. In an alternative or additional embodiment the GLP-1 receptor agonist is for use in treating hypoglycaemia or hypoglycaemic episodes associated with insulin therapy of type 1 diabetes.

In an alternative or additional embodiment the GLP-1 receptor agonist is for use in treating hypoglycaemia or hypoglycaemic episodes associated with insulin therapy of type 1 diabetes.

The treatment or prevention of hypoglycaemia or hypoglycaemic episodes in patients suffering from type 1 diabetes may comprise or may consist of treating or preventing one or more symptom selected from the group consisting of: hypoglycaemia; overall hypoglycaemic episodes; nocturnal hypoglycaemic episodes; and daytime hypoglycaemic episodes.

In an alternative or additional embodiment the treatment or prevention of hypoglycaemia or hypoglycaemic episodes in patients suffering from type 1 diabetes comprises or consists of treating or preventing one or more symptoms selected from the group consisting of: hypoglycaemia; overall hypoglycaemic episodes; nocturnal hypoglycaemic episodes; and daytime hypoglycaemic episodes.

Preferably, the severe hypoglycaemia (which can be defined as a blood glucose level of 40 mg/dL (2.2 mmol/L) or 50 mg/dL (2.8 mmol/L)) is prevented. Preferably mild-to-moderate hypoglycaemia (which can be defined as 40-69 mg/dL (2.2-3.8 mmol/L)) is prevented.

### Diabetes

In an alternative or additional embodiment the type 1 diabetes exhibits complete or partial insulin deficiency. By “exhibits partial insulin deficiency” we include that the individual produces at least some residual endogenous insulin. Such residual endogenous insulin may be provided by a residual β-cell function, which will decline further over time, leading to a “complete insulin deficiency” in Type 1 Diabetes Mellitus patients. Endogenous insulin production can be determined by measuring blood c-peptide. Any suitable method for measuring blood c-peptide known in the art can be used. However, in an alternative or additional embodiment, blood c-peptide is measured according to the method described in Hideshi Kuzuya, Petra M Blix, David L Horwitz, Donald F Steiner and Arthur H Rubenstein, Determination of Free and Total Insulin and C-Peptide in Insulin-treated Diabetics, Diabetes, 1977, 26(1):22-29, which is incorporated by reference herein. Various method of c-peptide measurements and their relative merits are discussed in Bonser A M and Garcia-Webb P., C-peptide measurement: methods and clinical utility, Crit Rev Clin Lab Sci. 1984; 19(4):297-352, which is also incorporated by reference herein.

In an alternative or additional embodiment the type 1 diabetes exhibits absolute, or substantially absolute, insulin deficiency. By “exhibits absolute, or substantially absolute, insulin deficiency” we include that the individual produces no, or ≦1% of normal levels of endogenous insulin (for example, as determined by measuring C-peptide levels). C-peptide levels are normal if ≧300 pmol/L and a T1DM patient has a level of <300 mmol/L.

By “diabetes” we include the exhibition of one or more of the following symptoms:

i) HbA_{1C} ≧6.5%. The test should be performed in a laboratory using a method that is NGSP certified and standardized to the DCCT assay (In the absence of an unequivocal outcome, result should be confirmed by repeat testing);

ii) Fasting plasma glucose (FPG) ≧126 mg/dL (7.0 mmol/L). Fasting is defined as no caloric intake for at least 8 h (In the absence of unequivocal hyperglycaemia, result should be confirmed by repeat testing);

iii) 2-h plasma glucose ≧200 mg/dL (11.1 mmol/L) during an oral glucose tolerance test (OGTT). The test should be performed as described by the WHO, using a glucose load containing the equivalent of 75 g anhydrous glucose dissolved in water (In the absence of unequivocal hyperglycaemia, result should be confirmed by repeat testing); and

iv) In a patient with classic symptoms of hyperglycaemia or hyperglycaemic crisis, a random plasma glucose ≧200 mg/dL (11.1 mmol/L) (In the absence of unequivocal hyperglycaemia, result should be confirmed by repeat testing).

If two different tests are available in an individual and the results are discordant, the test whose result is above the diagnostic cut point should be repeated, and the diagnosis is made on the basis of the confirmed test. That is, if a patient meets the diabetes criterion of the A1C (two results ≧6.5%) but not the FPG (<126 mg/dL or 7.0 mmol/L), or vice versa, that person should be considered to have diabetes.

By “type 1 diabetes” we include diabetics exhibiting one or more of the following: Islet Cell Antibodies (ICA, against cytoplasmic proteins in the beta cell); antibodies to Glutamic Acid Decarboxylase (GAD-65); Insulin Autoantibodies (IAA); IA-2A, to protein tyrosine phosphatise; diabetic ketoacidosis; insulin treatment from diagnosis; family history (blood relatives with type 1 diabetes); and A blood C-peptide measurement indicative of type 1 diabetes. Preferably, by “type 1 diabetes” we include diabetics exhibiting one or more of the following: diabetic ketoacidosis; insulin treatment from diagnosis; family history (blood relatives with type 1 diabetes); and a blood C-peptide measurement indicative of type 1 diabetes.

By “blood C-peptide measurement indicative of type 1 diabetes” we include blood C-peptide measurements lower than the normal range for non-diabetic patients. In an alternative or additional embodiment by “blood C-peptide measurement indicative of type 1 diabetes” we include blood C-peptide measurements of less than 0.51 nanograms per millilitre (ng/mL) (0.17 nanomoles per litre (nmol/L)). Blood C-peptide measurements may be made with any suitable means known to the skilled person, however, In an alternative or additional embodiment blood C-peptide measurements are taken according to the method described in Hideshi Kuzuya, Petra M Blix, David L Horwitz, Donald F Steiner and Arthur H Rubenstein, Determination of Free and Total Insulin and C-Peptide in Insulin-treated Diabetics, Diabetes, 1977, 26(1):22-29 which are incorporated by reference herein. Various method of c-peptide measurements and their relative merits are discussed in Bonser A M and Garcia-Webb P., C-peptide measurement: methods and clinical utility, Crit Rev Clin Lab Sci. 1984; 19(4):297-352, which is also incorporated by reference herein.

Two major methods have been used to measure glucose. The first, still in use in some places, is a chemical method exploiting the nonspecific reducing property of glucose in a reaction with an indicator substance that changes color when reduced. Since other blood compounds also have reducing properties (e.g., urea, which can be abnormally high in uremic patients), this technique can produce erroneous readings in some situations (5 to 15 mg/dL has been reported). The more recent technique, using enzymes specific to glucose, is less susceptible to this kind of error. The two most common employed enzymes are glucose oxidase and hexokinase.

In either case, the chemical system is commonly contained on a test strip which is inserted into a meter, and then has a blood sample applied. Test-strip shapes and their exact chemical composition vary between meter systems and cannot be interchanged.

Formerly, some test strips were read (after timing and wiping away the blood sample) by visual comparison against a color chart printed on the vial label. Strips of this type are still used for urine glucose readings, but for blood glucose levels they are obsolete. Their error rates were, in any case, much higher.

Urine glucose readings, however taken, are much less useful. In properly functioning kidneys, glucose does not appear in urine until the renal threshold for glucose has been exceeded. This is substantially above any normal glucose level, and is evidence of an existing severe hyperglycaemic condition. However, as urine is stored in the bladder, any glucose in it might have been produced at any time since the last time the bladder was emptied. Since metabolic conditions change rapidly, as a result of any of several factors, this is delayed news and gives no warning of a developing condition. Blood glucose monitoring is far preferable, both clinically and for home monitoring by patients. Healthy urine glucose levels were first standardized and published in 1965 by Hans Renschler.

In an alternative or additional embodiment the GLP-1 receptor agonist is for use in the treatment or prevention of one or more additional symptoms associated with insulin therapy of type 1 diabetes. By “one or more additional symptoms associated with insulin therapy of type 1 diabetes” we include any adverse effect seen in type 1 diabetes patients receiving insulin therapy compared to placebo not receiving insulin therapy other than hypoglycaemia or hypoglycaemic episodes. In an alternative or additional embodiment the one or more additional symptoms associated with insulin therapy of type 1 diabetes are selected from the group consisting of: reduced glycaemic control; insulin deficiency; and obesity. By “reduced glycaemic control” we include that HbA_{1c} levels are higher compared to placebo. HbA_{1c} levels can be measured by any suitable method known in the art. However, in an alternative or additional embodiment HbA_{1c} is measured using a Bio-Rad high-performance liquid chromatography method. By “insulin deficiency” we include the dose/amount of insulin required by the patient to maintain target blood glucose and/or HbA_{1c} levels. By “obesity” we mean accumulation of body fat associated with, or brought about by, insulin therapy.

The treatment with a derivative according to this invention may also be combined with a surgery that influences the glucose levels, and/or lipid homeostasis such as gastric banding or gastric bypass.

### Nocturnal Hypoglycaemia

Nocturnal hypoglycaemia is common in patients with type 1 diabetes and may be asymptomatic. In an alternative or additional embodiment, nocturnal hypoglycaemia is common in patients with type 1 diabetes and is usually asymptomatic. Nocturnal hyperinsulinemia frequently occurs with insulin therapy, and although blood glucose levels are often low during sleep, they are seldom measured routinely. Almost 50% of all episodes of severe hypoglycaemia occur at night during sleep. Such episodes can cause convulsions and coma and have been implicated as a precipitating factor in cardiac arrhythmias resulting in sudden death—the “dead-in-bed syndrome”. Nocturnal hypoglycaemia seems to have no immediate detrimental effect on cognitive function; however, on the following day, mood and well-being may be adversely affected. Recurrent exposure to nocturnal hypoglycaemia may impair cognitive function; other substantial long-term morbidity includes the development of acquired hypoglycaemia syndromes, such as impaired awareness of hypoglycaemia, through the putative effect of unsuspected recurrent episodes of nocturnal hypoglycaemia (see, for example, Endocr Pract. 2003 November-December; 9(6):530-43. Nocturnal hypoglycaemia: clinical manifestations and therapeutic strategies toward prevention. Allen K V, Frier B M).

Disturbances in hormonal counterregulation may be the main reason why many type 1 diabetic patients are asymptomatic during nighttime hypoglycaemia. While it is known that sleep attenuates counterregulatory responses to hypoglycaemia, the influence of the time of day on hormonal counterregulation regulation remains obscure (see, for example, Metabolism. 2004 July; 53(7):894-8. Differences between nighttime and daytime hypoglycaemia counterregulation in healthy humans. Merl V, Kern W, Peters A, Oltmanns K M, Gais S, Born J, Fehm H L, Schultes).

In an alternative or additional embodiment the GLP-1 receptor agonist is capable of reducing overall nocturnal hypoglycaemic episodes.

By “reducing overall nocturnal hypoglycaemic episodes” we include reducing the number of nocturnal hypoglycaemic episodes experienced over a period of time, for example, over one day, one night, one day and night (i.e. 24 hours), seven days, four weeks, 42 weeks or more compared to placebo not undergoing treatment with a, or the, GLP-1 receptor agonist of the invention.

In an alternative or additional embodiment the GLP-1 receptor agonist is capable of reducing overall nocturnal hypoglycaemic episodes by at least 25%, for example, 30%, 35%, or 40%. In an alternative or additional embodiment the GLP-1 receptor agonist is capable of reducing overall nocturnal hypoglycaemic episodes by at least 31%. In an alternative or additional embodiment the GLP-1 receptor agonist is capable of reducing overall nocturnal hypoglycaemic episodes by at least 35%. In an alternative or additional embodiment the GLP-1 receptor agonist is capable of reducing overall nocturnal hypoglycaemic episodes by at least 39%. In an alternative or additional embodiment the GLP-1 receptor agonist is capable of reducing overall nocturnal hypoglycaemic episodes by at least 31%, when said dose of GLP-1 receptor agonist is 0.6 mg/day. In an alternative or additional embodiment the GLP-1 receptor agonist is capable of reducing overall nocturnal hypoglycaemic episodes by at least 35% %, when said dose of GLP-1 receptor agonist is 1.2 mg/day. In an alternative or additional embodiment the GLP-1 receptor agonist is capable of reducing overall nocturnal hypoglycaemic episodes by at least 39%, when said dose of GLP-1 receptor agonist is 1.8 mg/day.

In an alternative or additional embodiment the GLP-1 receptor agonist is capable of reducing the duration of nocturnal hypoglycaemic episodes. By “reducing the duration of nocturnal hypoglycaemic episodes” we include reducing_the duration of nocturnal hypoglycaemic episodes experienced compared to placebo not undergoing treatment with a, or the, GLP-1 receptor agonist of the invention.

### GLP-1 Receptor Agonist

In an alternative or additional embodiment the GLP-1 receptor agonist comprises no more than 5, such as no more than 4 or no more than 3, amino acid residues which have been substituted, inserted or deleted as compared to GLP-1(7-37). The GLP-1 receptor agonist may be a GLP-1 fragment, derivative or analogue. The GLP-1 receptor agonist may be suitable for once daily administration. The GLP-1 receptor agonist may be suitable for once weekly administration. The GLP-1 receptor agonist may be selected from the group consisting of: GLP-1(7-37); GLP-1(7-36) amide; Exenatide; Exenatide LAR; Liraglutide; Semaglutide; Taspoglutide; Albiglutide; Lixisenatide; and Dulaglutide. The GLP-1 receptor agonist may be Liraglutide or Semaglutide. The GLP-1 receptor agonist may be liraglutide. The GLP-1 receptor agonist may be semaglutide. The GLP-1 receptor agonist may be a long-acting GLP-1 derivative or analogue. The long-acting GLP-1 derivative or analogue may be selected from the group consisting of: Liraglutide; Semaglutide; Taspoglutide; Albiglutide; Taspoglutide; Dulaglutide; and Exenatide LAR. The long-acting GLP-1 derivative or analogue may be Liraglutide. The long-acting GLP-1 derivative or analogue may be Semaglutide.

In an alternative or additional embodiment the GLP-1 receptor agonist is suitable for once daily administration. By “suitable for once daily administration” we include that the GLP-1 receptor agonist is suitable for treating diabetes (type 1 diabetes and/or type 2 diabetes) by administration once every 24 hours or once every approximately 24 hours. By “suitable for administration once every approximately 24 hours” we include that the GLP-1 receptor agonist is suitable for treating diabetes (type 1 diabetes and/or type 2 diabetes) by administration once every 24 hours±6 hours or less. Hence, The GLP-1 receptor agonist may be suitable for administration every 24 hours±5 hours or less. Hence, The GLP-1 receptor agonist may be suitable for administration every 24 hours±4 hours or less. Hence, The GLP-1 receptor agonist may be suitable for administration every 24 hours±3 hours or less. Hence, The GLP-1 receptor agonist may be suitable for administration every 24 hours±2 hours or less. Hence, The GLP-1 receptor agonist may be suitable for administration every 24 hours±1 hour or less. Hence, The GLP-1 receptor agonist may be suitable for administration every or 24 hours±30 minutes or less. In an alternative or additional embodiment, the GLP-1 receptor agonist of the invention has a duration of action suitable for once daily administration in the treatment of diabetes (type 1 diabetes and/or type 2 diabetes). However, in an alternative or additional embodiment, by “GLP-1 receptor agonist has a duration of action suitable for once daily administration in the treatment of diabetes” we include GLP-1 receptor agonists with a plasma half-life (T_{1/2}) of between 30 minutes and 10 hours. In an alternative or additional embodiment the GLP-1 receptor agonist has a plasma half-life (T_{1/2}) of between 1 hour and 8 hours. In an alternative or additional embodiment the GLP-1 receptor agonist has a plasma half-life (T_{1/2}) of between 2 hours and 6 hours. In an alternative or additional embodiment the GLP-1 receptor agonist has a plasma half-life (T_{1/2}) of between 2 hours and 4 hours. In an alternative or additional embodiment the GLP-1 receptor agonist has a plasma half-life (T_{1/2}) of between 2 hours and 3 hours. Alternatively, in an alternative or additional embodiment, by “GLP-1 receptor agonist has a duration of action suitable for once daily administration in the treatment of diabetes” we include GLP-1 receptor agonists with a plasma half-life (T_{1/2}) of between 30 minutes and 20 hours in humans. In an alternative or additional embodiment the GLP-1 receptor agonist has a plasma half-life (T_{1/2}) of between 8 hours and 18 hours, such as between 9 hours and 16 hours or between 10 hours and 15 hours, in humans. In an alternative or additional embodiment the GLP-1 receptor agonist has a plasma half-life (T_{1/2}) of between 1 hour and 8 hours, such as between 2 hours and 6 hours or between 2 hours and 4 hours, in humans. In an alternative or additional embodiment, the GLP-1 receptor agonist of the invention is selected from the group consisting of exenatide and lixisenatide.

In an alternative or additional embodiment the GLP-1 receptor agonist is suitable for once weekly administration. By “suitable for once weekly administration” we include that the GLP-1 receptor agonist is suitable for treating diabetes (type 1 diabetes and/or type 2 diabetes) by administration once every 168 hours or once every approximately 168 hours. By “suitable for administration once every approximately 168 hours” we include that the GLP-1 receptor agonist is suitable for treating diabetes (type 1 diabetes and/or type 2 diabetes) by administration once every 168 hours±48 hours or less. In an alternative or additional embodiment the GLP-1 receptor agonist is suitable for treating diabetes (type 1 diabetes and/or type 2 diabetes) by administration once every 168 hours±36 hours or less. In an alternative or additional embodiment the GLP-1 receptor agonist is suitable for treating diabetes (type 1 diabetes and/or type 2 diabetes) by administration once every 168 hours±24 hours or less. In an alternative or additional embodiment the GLP-1 receptor agonist is suitable for treating diabetes (type 1 diabetes and/or type 2 diabetes) by administration once every 168 hours±18 hours or less. In an alternative or additional embodiment the GLP-1 receptor agonist is suitable for treating diabetes (type 1 diabetes and/or type 2 diabetes) by administration once every 168 hours±15 hours or less. In an alternative or additional embodiment the GLP-1 receptor agonist is suitable for treating diabetes (type 1 diabetes and/or type 2 diabetes) by administration once every 168 hours±9 hours or less. In an alternative or additional embodiment the GLP-1 receptor agonist is suitable for treating diabetes (type 1 diabetes and/or type 2 diabetes) by administration once every 168 hours±6 hours or less. In an alternative or additional embodiment the GLP-1 receptor agonist is suitable for treating diabetes (type 1 diabetes and/or type 2 diabetes) by administration once every 168 hours±3 hours or less. In an alternative or additional embodiment the GLP-1 receptor agonist is suitable for treating diabetes (type 1 diabetes and/or type 2 diabetes) by administration once every 168 hours±1 hour or less. In an alternative or additional embodiment, the GLP-1 receptor agonist of the invention has a duration of action suitable for once weekly administration in the treatment of diabetes (type 1 diabetes and/or type 2 diabetes). In an alternative or additional embodiment, by “GLP-1 receptor agonist has a duration of action suitable for once weekly administration in the treatment of diabetes” we include GLP-1 receptor agonists with a plasma half-life (T_{1/2}) of between 2 days and 15 days. In an alternative or additional embodiment the GLP-1 receptor agonist has a plasma half-life (T_{1/2}) of between 3 days and 12 days. In an alternative or additional embodiment the GLP-1 receptor agonist has a plasma half-life (T_{1/2}) of between 4 days and 9 days. In an alternative or additional embodiment the GLP-1 receptor agonist has a plasma half-life (T_{1/2}) of between 4 days and 7 days. In an alternative or additional embodiment, the GLP-1 receptor agonist of the invention is selected from the group consisting of dulaglutide, albiglutide, exenatide LAR and taspoglutide.

In an alternative or additional embodiment the plasma half-life of the GLP-1 receptor agonist is determined in humans. Plasma half-life can be determined by any suitable means known in the art. However, In an alternative or additional embodiment, circulating half-life is determined using the method described in Deacon C F, Nauck M A, Meier J, Hucking K, Hoist J J: Degradation of endogenous and exogenous gastric inhibitory polypeptide in healthy and in type 2 diabetic subjects as revealed using a new assay for the intact peptide, J Clin Endocrinol Metab 85:3575-3581, 2000, which is incorporated by reference herein. In an alternative embodiment, half-life is determined through a pharmacodynamic or pharmacokinetic assay described in the present Examples section (Example C). The terms “half-life”, “plasma half-life”, “terminal half-life”, “terminal plasma half-life”, “t½”, and “T½” may be used interchangeably herein.

Duration of action is a function of several parameters including plasma half-life, the time to equilibrate between plasma and target compartments, and the off rate of the drug from its biological target.

In an alternative or additional embodiment the GLP-1 receptor agonist is a GLP-1 fragment, derivative or analogue, e.g. a GLP-1 analogue or GLP-1 derivative.

The term “GLP-1 peptide” as used herein refers to the human Glucagon-Like Peptide-1 (GLP-1(7-37)), the sequence of which is included in the sequence listing as SEQ ID NO: 1, or an analogue thereof. The peptide having the sequence of SEQ ID NO: 1 may also be designated human GLP-1 or “native” GLP-1, alternatively native” GLP-1(7-37).

In an alternative or additional embodiment the term “GLP-1 analogue” or “analogue of GLP-1” as used herein may refer to a peptide, or a compound, which is a variant of GLP-1(7-37) (SEQ ID NO: 1): HAEGTFTSDVSSYLEGQAAKEFIAWLVKGRG [SEQ ID NO: 1]. In an alternative or additional embodiment the term “variant” when used in connection with GLP-1, such as GLP-1(7-37), refers to a peptide which comprises one or more amino acid residue changes, such as substitutions, additions or deletions, compared to human GLP-1(7-37) (SEQ ID NO: 1). In the sequence listing, the first amino acid residue of SEQ ID NO: 1 (histidine) is assigned no. 1. However, as used herein and in what follows—according to established practice in the art—this histidine residue is referred to as no. 7, and subsequent amino acid residues are numbered accordingly, ending with glycine no. 37. Therefore, generally, any reference herein to an amino acid residue number or a position number of the GLP-1(7-37) sequence is to the sequence starting with His at position 7 and ending with Gly at position 37. GLP-1 receptor agonists, such as GLP-1 analogues of the derivatives of the invention, may be described by reference to i) the number of the amino acid residue in native GLP-1(7-37) which corresponds to the amino acid residue which is changed (i.e., the corresponding position in native GLP-1), and to ii) the actual change. In other words, a GLP-1 analogue may be a GLP-1(7-37) peptide in which a number of amino acid residues have been changed when compared to native GLP-1(7-37) (SEQ ID NO: 1). These changes may represent, independently, one or more amino acid substitutions, additions, and/or deletions. Similarly, in an alternative or additional embodiment, the term “analogue” as used herein, and unless otherwise stated, refers to human GLP-1(7-37) in which one or more amino acid residues have been changed (i.e. substituted, added, and/or deleted) when compared to native GLP-1(7-37) (SEQ ID NO: 1); t. These changes may represent, independently, one or more amino acid

The following are non-limiting examples of suitable nomenclature for GLP-1 receptor agonists, such as analogue nomenclature.

GLP-1 receptor agonists, such as analogues, “comprising” certain specified changes may comprise further changes, when compared to SEQ ID NO: 1. In an alternative or additional embodiment the GLP-1 receptor agonist, such as analogue, “has” the specified changes.

As is apparent from the above examples, amino acid residues may be identified by their full name, their one-letter code, and/or their three-letter code. These three ways are fully equivalent.

The expressions “a position equivalent to” or “corresponding position” may be used to characterise the site of change in a variant GLP-1(7-37) sequence by reference to native GLP-1(7-37) (SEQ ID NO: 1). Equivalent or corresponding positions, as well as the number of changes, are easily deduced, e.g. by simple handwriting and eyeballing; and/or a standard protein or peptide alignment program may be used, such as “align” which is based on a Needleman-Wunsch algorithm. This algorithm is described in Needleman, S. B. and Wunsch, C. D., (1970), Journal of Molecular Biology, 48: 443-453, and the align program by Myers and W. Miller in “Optimal Alignments in Linear Space” CABIOS (computer applications in the biosciences) (1988) 4:11-17. For the alignment, the default scoring matrix BLOSUM62 and the default identity matrix may be used, and the penalty for the first residue in a gap may be set at −12, or preferably at −10, and the penalties for additional residues in a gap at −2, or preferably at −0.5.

In case of non-natural amino acids such as Imp and/or Aib being included in the sequence, these may, for alignment purposes, be replaced with, e.g., X. If desired, X can later be manually corrected.

The term “peptide”, as e.g. used in the context of the GLP-1 receptor agonists, such as GLP-1 analogues of the derivatives of the invention, refers to a compound which comprises a series of amino acids interconnected by amide (or peptide) bonds. The peptides of the invention comprise at least five constituent amino acids connected by peptide bonds. In an alternative or additional embodiment the peptide comprises at least 10, preferably at least 15, more preferably at least 20, even more preferably at least 25, or most preferably at least 28 amino acids. In an alternative or additional embodiment the peptide is composed of at least five constituent amino acids, preferably composed of at least 10, at least 15, at least 20, at least 25, or most preferably composed of at least 28 amino acids. In an alternative or additional embodiment the peptide is a) composed of, or b) consists of, i) 29, ii) 30, iii) 31, or iv) 32 amino acids. In an alternative or additional embodiment the peptide consists of amino acids interconnected by peptide bonds.

Amino acids are molecules containing an amine group and a carboxylic acid group, and, optionally, one or more additional groups, often referred to as a side chain. The term “amino acid” includes proteinogenic (or natural) amino acids (amongst those the 20 standard amino acids), as well as non-proteinogenic (or non-natural) amino acids. Proteinogenic amino acids are those which are naturally incorporated into proteins. The standard amino acids are those encoded by the genetic code. Non-proteinogenic amino acids are either not found in proteins, or not produced by standard cellular machinery (e.g., they may have been subject to post-translational modification). Non-limiting examples of non-proteinogenic amino acids are Aib (α-aminoisobutyric acid), des-amino-histidine (alternative name imidazopropionic acid, abbreviated Imp), as well as the D-isomers of the proteinogenic amino acids.

In an alternative or additional embodiment the GLP-1 receptor agonist comprises no more than 5, such as no more than 4 or no more than 3, amino acid residues which have been substituted, inserted or deleted as compared to GLP-1(7-37). In an alternative or additional embodiment the GLP-1 receptor agonist comprises no more than 4 amino acid residues which are not encoded by the genetic code.

As used herein, and in what follows, all amino acids of the GLP-1 receptor agonist or GLP-1 peptide for which the optical isomer is not stated is to be understood to mean the L-isomer (unless otherwise specified).

The GLP-1 receptor agonists, such as GLP-1 derivatives and analogues, of the invention have GLP-1 activity. This term refers to the ability to bind to the GLP-1 receptor and initiate a signal transduction pathway resulting in insulinotropic action or other physiological effects as is known in the art. For example, GLP-1 receptor agonists, such as the analogues and derivatives of the invention, can be tested for GLP-1 activity using the assay described in the Examples section herein.

The term “derivative” as used herein in the context of a GLP-1 receptor agonist, such as a GLP-1 peptide or analogue, means a chemically modified GLP-1 receptor agonist or GLP-1 peptide, in which one or more substituents have been covalently attached to the peptide. The substituent may also be referred to as a side chain.

In an alternative or additional embodiment the side chain is capable of forming non-covalent aggregates with albumin, thereby promoting the circulation of the GLP-1 receptor agonist (e.g. derivative) with the blood stream, and also having the effect of protracting the time of action of the GLP-1 receptor agonist (e.g. derivative), due to the fact that the aggregate of the GLP-1 receptor agonist (e.g. GLP-1-derivative) and albumin is only slowly disintegrated to release the active pharmaceutical ingredient. Thus, the substituent, or side chain, as a whole is preferably referred to as an albumin binding moiety.

In an alternative or additional embodiment the side chain has at least 10 carbon atoms, or at least 15, 20, 25, 30, 35, or at least 40 carbon atoms. In an alternative or additional embodiment the side chain may further include at least 5 hetero atoms, in particular 0 and N, for example at least 7, 9, 10, 12, 15, 17, or at least 20 hetero atoms, such as at least 1, 2, or 3 N-atoms, and/or at least 3, 6, 9, 12, or 15 O-atoms.

In an alternative or additional embodiment the albumin binding moiety comprises a portion which is particularly relevant for the albumin binding and thereby the protraction, which portion may accordingly be referred to as a protracting moiety. The protracting moiety may be near, preferably at, the terminal (or distal, or free) end of the albumin binding moiety, relative to its point of attachment to the peptide.

In an alternative or additional embodiment the albumin binding moiety comprises a portion between the protracting moiety and the point of attachment to the peptide, which portion may be referred to as a linker, linker moiety, spacer, or the like. The linker may be optional, and hence in that case the albumin binding moiety may be identical to the protracting moiety.

In an alternative or additional embodiment the albumin binding moiety and/or the protracting moiety is lipophilic, and/or negatively charged at physiological pH (7.4).

The albumin binding moiety, the protracting moiety, or the linker may be covalently attached to a lysine residue of the GLP-1 receptor agonist or GLP-1 peptide by acylation, i.e., via an amide bond formed between a carboxylic acid group thereof (of the albumin binding moiety, the protracting moiety, or the linker) and an amino group of the lysine residue. Additional or alternative conjugation chemistry includes alkylation, ester formation, or amide formation, or coupling to a cysteine residue, such as by maleimide or haloacetamide (such as bromo-/fluoro-/iodo-) coupling.

In an alternative or additional embodiment, an active ester of the albumin binding moiety, preferably comprising a protracting moiety and a linker, is covalently linked to an amino group of a lysine residue, preferably the epsilon amino group thereof, under formation of an amide bond, as explained above.

Unless otherwise stated, when reference is made to an acylation of a lysine residue, it is understood to be to the epsilon-amino group thereof.

The term “fatty acid” refers to aliphatic monocarboxylic acids having from 4 to 28 carbon atoms, it is preferably un-branched, and it may be saturated or unsaturated.

The term “fatty diacid” refers to fatty acids as defined above but with an additional carboxylic acid group in the omega position. Thus, fatty diacids are dicarboxylic acids.

In an alternative or additional embodiment, each of the two linkers of the GLP-1 receptor agonist (e.g. derivative) of the invention may comprise the following first linker element:

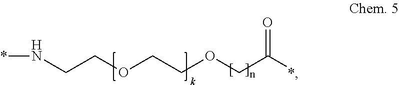

wherein k is an integer in the range of 1-5, and n is an integer in the range of 1-5.

In an alternative or additional embodiment, when k=1 and n=1, this linker element may be designated OEG, or a di-radical of 8-amino-3,6-dioxaoctanic acid, and/or it may be represented by the following formula:

*—NH—(CH₂)₂—O—(CH₂)₂—O—CH₂—CO—*. Chem. 5a:

In an alternative or additional embodiment each linker of the GLP-1 receptor agonist (e.g. derivative) of the invention may further comprise, independently, a second linker element, preferably a Glu di-radical, such as Chem. 6:

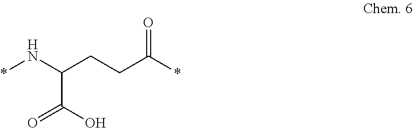 [SMILES: *NC(CCC(*)=O)C(=O)O]

wherein the Glu di-radical may be included p times, where p is an integer in the range of 1-3. Chem. 6 may also be referred to as gamma-Glu, or briefly gGlu, due to the fact that it is the gamma carboxy group of the amino acid glutamic acid which is here used for connection to another linker element, or to the epsilon-amino group of lysine. As explained above, the other linker element may, for example, be another Glu residue, or an OEG molecule. The amino group of Glu in turn forms an amide bond with the carboxy group of the protracting moiety, or with the carboxy group of, e.g., an OEG molecule, if present, or with the gamma-carboxy group of, e.g., another Glu, if present. As explained above, in an alternative or additional embodiment, the GLP-1 receptor agonists (e.g. the GLP-1 derivatives) of the present invention are double-acylated, i.e. two albumin binding moieties are covalently attached to the GLP-1 receptor agonist, e.g. the GLP-1 peptide. In an alternative or additional embodiment the two albumin binding moieties (i.e. the entire side chains) are similar, preferably substantially identical, or, most preferably, identical. In an alternative or additional embodiment the two protracting moieties are similar, preferably substantially identical, or, most preferably, identical. In an alternative or additional embodiment the two linkers are similar, preferably substantially identical, or, most preferably identical. The term “substantially identical” includes differences from identity which are due to formation of one or more salts, esters, and/or amides; preferably formation of one or more salts, methyl esters, and simple amides; more preferably formation of no more than two salts, methyl esters, and/or simple amides; even more preferably formation of no more than one salt, methyl ester, and/or simple amide; or most preferably formation of no more than one salt.

In the context of chemical compounds such as the albumin binding moieties, protracting moieties, and linkers, similarity and/or identity may be determined using any suitable computer program and/or algorithm known in the art.

For example, the similarity of two protracting moieties, two linkers, and/or two entire side chains may suitably be determined using molecular fingerprints. Fingerprints is a mathematical method of representing a chemical structure (see e.g. Chemoinformatics: A textbook, Johann Gasteiger and Thomas Engel (Eds), Wiley-VCH Verlag, 2003). Examples of suitable fingerprints include, without limitation, UNITY fingerprints, MDL fingerprints, and/or ECFP fingerprints, such as ECFP_6 fingerprints (ECFP stands for extended-connectivity fingerprints).

In an alternative or additional embodiment the two protracting moieties, the two linkers, and/or the two entire side chains are represented as a) ECFP_6 fingerprints; b) UNITY fingerprints; and/or c) MDL fingerprints.

The Tanimoto coefficient is preferably used for calculating the similarity of the two fingerprints, whether a), b), or c) is used.

In an alternative or additional embodiment whether a), b), or c) is used, the two protracting moieties, the two linkers, and/or the two entire side chains, respectively, have a similarity of at least 0.5 (50%); preferably at least 0.6 (60%); more preferably at least 0.7 (70%), or at least 0.8 (80%); even more preferably at least 0.9 (90%); or most preferably at least 0.99 (99%), such as a similarity of 1.0 (100%).

UNITY fingerprints may be calculated using the programme SYBYL (available from Tripos, 1699 South Hanley Road, St. Louis, Mo. 63144-2319 USA). ECFP_6 and MDL fingerprints may be calculated using the programme Pipeline Pilot (available from Accelrys Inc., 10188 Telesis Court, Suite 100, San Diego, Calif. 92121, USA).

For more details, see for example J. Chem. Inf. Model. 2008, 48, 542-549; J. Chem. Inf. Comput. Sci. 2004, 44, 170-178; J. Med. Chem. 2004, 47, 2743-2749; J. Chem. Inf. Model. 2010, 50, 742-754; as well as SciTegic Pipeline Pilot Chemistry Collection: Basic Chemistry User Guide, March 2008, SciTegic Pipeline Pilot Data Modeling Collection, 2008—both from Accelrys Software Inc., San Diego, US, and the guides:

http://www.tripos.com/tripos_resources/fileroot/pdfs/Unity_111408.pdf, and

http://www.tripos.com/data/SYBYL/SYBYL_072505. pdf.

An example of a similarity calculation is inserted hereinbelow, in which a known entire side chain of a known GLP-1 derivative was compared with a methyl ester thereof:

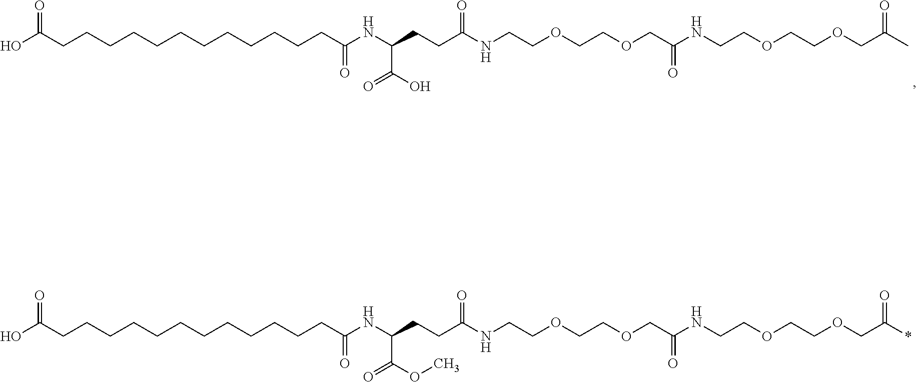 [SMILES: CC(=O)COCCOCCNC(=O)COCCOCCNC(=O)CC[C@H](NC(=O)CCCCCCCCCCCCC(=O)O)C(=O)O ; *C(=O)COCCOCCNC(=O)COCCOCCNC(=O)CC[C@H](NC(=O)CCCCCCCCCCCCC(=O)O)C(=O)OC]

Using a) ECFP_6 fingerprints the similarity is 0.798, using b) UNITY fingerprints the similarity is 0.957; and using MDL fingerprints the similarity is 0.905.

In case of two identical side chains (albumin binding moieties) the GLP-1 receptor agonist (e.g. the derivative) may be designated symmetrical.

In an alternative or additional embodiment the similarity coefficient is at least 0.80, preferably at least 0.85, more preferably at least 0.90, even more preferably at least 0.95, or most preferably at least 0.99.

The GLP-1 receptor agonists, such as derivatives, of the invention may exist in different stereoisomeric forms having the same molecular formula and sequence of bonded atoms, but differing only in the three-dimensional orientation of their atoms in space. The stereoisomerism of the exemplified GLP-1 receptor agonists, such as derivatives, of the invention is indicated in the experimental section, in the names as well as the structures, using standard nomenclature. Unless otherwise stated the invention relates to all stereoisomeric forms of the claimed GLP-1 receptor agonist (e.g. derivative).

The concentration in plasma of the GLP-1 receptor agonists, such as GLP-1 derivatives, of the invention may be determined using any suitable method. For example, LC-MS (Liquid Chromatography Mass Spectroscopy) may be used, or immunoassays such as RIA (Radio Immuno Assay), ELISA (Enzyme-Linked Immuno Sorbent Assay), and LOCI (Luminescence Oxygen Channeling Immunoasssay). General protocols for suitable RIA and ELISA assays are found in, e.g., WO 2009/030738 on p. 116-118. A preferred assay is the LOCI assay described in the Examples section herein.

The GLP-1 receptor agonists, such as the derivatives, analogues, and intermediate products, of the invention may be in the form of a pharmaceutically acceptable salt, amide, or ester. Salts are e.g. formed by a chemical reaction between a base and an acid, e.g.: 2NH₃+H₂SO₄→(NH₄)₂SO₄. The salt may be a basic salt, an acid salt, or it may be neither nor (i.e. a neutral salt). Basic salts produce hydroxide ions and acid salts hydronium ions in water. The salts of the GLP-1 receptor agonists, such as derivatives, of the invention may be formed with added cations or anions between anionic or cationic groups, respectively. These groups may be situated in the peptide moiety, and/or in the side chain of the GLP-1 receptor agonists, such as derivatives, of the invention.

Non-limiting examples of anionic groups of the GLP-1 receptor agonists, such as derivatives, of the invention include free carboxylic groups in the side chain, if any, as well as in the peptide moiety. The peptide moiety often includes a free carboxylic acid group at the C-terminus, and it may also include free carboxylic groups at internal acid amino acid residues such as Asp and Glu. Non-limiting examples of cationic groups in the peptide moiety include the free amino group at the N-terminus, if present, as well as any free amino group of internal basic amino acid residues such as His, Arg, and Lys.

The ester of the GLP-1 receptor agonists, such as derivatives, of the invention may, e.g., be formed by the reaction of a free carboxylic acid group with an alcohol or a phenol, which leads to replacement of at least one hydroxyl group by an alkoxy or aryloxy group. The ester formation may involve the free carboxylic group at the C-terminus of the peptide, and/or any free carboxylic group in the side chain.

The amide of the GLP-1 receptor agonists, such as derivatives, of the invention may, e.g., be formed by the reaction of a free carboxylic acid group with an amine or a substituted amine, or by reaction of a free or substituted amino group with a carboxylic acid. The amide formation may involve the free carboxylic group at the C-terminus of the peptide, any free carboxylic group in the side chain, the free amino group at the N-terminus of the peptide, and/or any free or substituted amino group of the peptide in the peptide and/or the side chain.

In an alternative or additional embodiment the GLP-1 receptor agonist (e.g. the peptide or derivative) is in the form of a pharmaceutically acceptable salt. In an alternative or additional embodiment the GLP-1 receptor agonist (e.g. derivative) is in the form of a pharmaceutically acceptable amide, preferably with an amide group at the C-terminus of the peptide. In an alternative or additional embodiment the GLP-1 receptor agonist (e.g. peptide or derivative) is in the form a pharmaceutically acceptable ester.

In an alternative or additional embodiment the GLP-1 receptor agonist, such as the GLP-1 derivative or analogue, is selected from the group consisting of: GLP-1(7-37); GLP-1(7-36) amide; Exenatide (Byette); Exenatide LAR (BYDUREON®); Liraglutide (Victoza®); Semaglutide; Taspoglutide; Albiglutide; Lixisenatide (Sanofi); and Dulaglutide.

In an alternative or additional embodiment the GLP-1 receptor agonist, such as the GLP-1 derivative or analogue, is selected from the group consisting of: Liraglutide (Victoza®) and Semaglutide.

Liraglutide, a mono-acylated GLP-1 derivative for once daily administration which is marketed as of 2009 by Novo Nordisk A/S, is disclosed in WO 98/08871 Example 37.

WO 2006/097537 discloses additional GLP-1 receptor agonist, such as GLP-1 derivatives, including semaglutide (Example 4).

Exenatide is a synthetic version of exendin-4, a hormone found in the saliva of the Gila monster. It displays biological properties similar to GLP-1.

Dulaglutide is a GLP-1-Fc construct (GLP-1-linker-Fc from IgG4).

Lixisenatide is based on exendin-4(1-39) modified C-terminally with six Lys residues (see IDrugs, 2009 August; 12(8):503-13)).

Taspoglutide is the 8-(2-methylalanine)-35-(2-methylalanine)-36-L-argininamide derivative of the amino acid sequence 7-36 of human GLP-1.

Albiglutide is a recombinant human serum albumin (HSA)-GLP-1 hybrid protein, likely a GLP-1 dimer fused to HSA. The constituent GLP-1 receptor agonist or GLP-1 peptide is an analogue, in which Ala at position 8 has been substituted by Gly (see Curr Opin Mol Ther, 2009 October; 11(5):579-88]).

In an alternative or additional embodiment the GLP-1 receptor agonist is a long-acting GLP-1 receptor agonist. By “long-acting GLP-1 receptor agonist” we include GLP-1 receptor agonists with increased terminal half-life and/or decreased clearance compared to the wild type protein (SEQ ID NO: 1). In an alternative or additional embodiment the GLP-1 derivative or analogue is a long-acting GLP-1 derivative or analogue. By “long-acting GLP-1 derivative or analogue”, we include GLP-1 derivatives or analogues with increased terminal half-life and/or decreased clearance compared to the wild type protein (SEQ ID NO: 1).

Terminal plasma half-life is the time required to divide the plasma concentration by two after reaching pseudo-equilibrium. When the process of absorption is not a limiting factor, half-life is a hybrid parameter controlled by plasma clearance and extent of distribution. In contrast, when the process of absorption is a limiting factor, the terminal half-life reflects rate and extent of absorption and not the elimination process. The terminal half-life is especially relevant to multiple dosing regimens, because it controls the degree of drug accumulation, concentration fluctuations and the time taken to reach equilibrium.

In an alternative or additional embodiment the long-acting GLP-1 receptor agonist, such as the long-acting GLP-1 derivative or analogue, is selected from the group consisting of: Liraglutide (Victoza®); Semaglutide; Taspoglutide; Albiglutide; Taspoglutide; Dulaglutide; and Exenatide LAR. In an alternative or additional embodiment the long-acting GLP-1 receptor agonist, such as the long-acting GLP-1 derivative or analogue, is Liraglutide (Victoza™). In an alternative or additional embodiment the long-acting GLP-1 receptor agonist, such as the long-acting GLP-1 derivative or analogue, is Semaglutide.

In an alternative or additional embodiment the GLP-1 receptor agonist is semaglutide. Semaglutide is a mono-acylated GLP-1 derivative for once weekly administration disclosed in Example 4 of WO 2006/097537 which is incorporated herein by reference.

By “GLP-1 receptor agonist” we include any agent capable of binding to and/or activating the GLP-1 receptor. In an alternative or additional embodiment the terms “GLP-1 receptor agonist” and “GLP-1 agonist” are used interchangeably herein. In an alternative or additional embodiment the GLP-1 receptor is mammalian, preferably human. In an alternative or additional embodiment the GLP-1 receptor is the GLP-1 receptor derived from GLP-1 receptor precursor protein Entrez accession number NP_002053 (e.g., version NP_002053.3). A receptor agonist may be defined as a GLP-1 receptor agonist, such as an analogue, that binds to a receptor and elicits a response typical of the natural ligand. A full agonist may be defined as one that elicits a response of the same magnitude as the natural ligand (see e.g. “Principles of Biochemistry”, A L Lehninger, D L Nelson, M M Cox, Second Edition, Worth Publishers, 1993, page 763). GLP-1 receptor binding can be determined by any suitable means known in the art. However, preferably, GLP-1 receptor binding and activation is determined by the ability to stimulate formation of cAMP in a cell line expressing the cloned human GLP-1 receptor, most preferably according to the method described in Examples section, below. A “full” GLP-1 receptor agonist may be defined as a GLP-1 receptor agonist which is capable of eliciting a magnitude of GLP-1 receptor response that is similar (for example, ±5%) or identical to native GLP-1, e.g. human GLP-1(7-37).

### Functional Properties

In a first functional alternative or additional embodiment, the GLP-1 receptor agonists, such as the derivatives, of the invention have a good potency (e.g., at least 50% that of the native protein (e.g. human GLP-1(7-37)), for example, at least 75%, at least 95%, at least 100%, at least 125%, at least 150% or at least 175% that of the native protein). Also, or alternatively, they bind very well to the GLP-1 receptor at a low concentration of albumin (≦0.0005% (wt/vol) albumin [e.g., HSA]). Preferably they are full GLP-1 receptor agonists as is reflected by their ability to bind strongly to the GLP-1 receptor combined with the capacity to activate the receptor. Also, or alternatively, in a third functional alternative or additional embodiment, they have improved pharmacokinetic properties compared to SEQ ID NO: 1. Also, or alternatively, in a fourth functional alternative or additional embodiment, they have a high oral bioavailability (for example, improved bioavailability compared to SEQ ID NO: 1). Also, or alternatively, in a fifth functional alternative or additional embodiment, they have good biophysical properties (for example, improved bioavailability compared to SEQ ID NO: 1).

The GLP-1 receptor agonists, such as the derivatives, of the invention, as well as the constituent GLP-1 peptides or analogues as such, are biologically active, or potent. In an alternative or additional embodiment, potency and/or activity refers to in vitro potency, i.e. performance in a functional GLP-1 receptor assay, more in particular to the capability of activating the human GLP-1 receptor. The in vitro potency may, e.g., be determined in a medium containing membranes expressing the human GLP-1 receptor, and/or in an assay with whole cells expressing the human GLP-1 receptor. For example, purified plasma membranes from a stable transfected cell line expressing the human GLP-1 receptor may be stimulated with the GLP-1 receptor agonist, such as the GLP-1 analogue or derivative, in question, and the potency of cAMP production measured, e.g. based on competition between endogenously formed cAMP and exogenously added biotin-labelled cAMP, which may be captured using a specific antibody, e.g. as described in Example C.

Also, or alternatively, the response of the human GLP-1 receptor may be measured in a reporter gene assay, e.g. in a stably transfected BHK cell line that expresses the human GLP-1 receptor and contains the DNA for the cAMP response element (CRE) coupled to a promoter and the gene for firefly luciferase (CRE luciferase). When cAMP is produced as a result of activation of the GLP-1 receptor this in turn results in the luciferase being expressed. Luciferase may be determined by adding luciferin, which by the enzyme is converted to oxyluciferin and produces bioluminescence, which is measured and is a measure of the in vitro potency. One non-limiting example of such an assay is described in Example C.

The term half maximal effective concentration (EC₅₀) generally refers to the concentration which induces a response halfway between the baseline and maximum, by reference to the dose response curve. EC₅₀ is used as a measure of the potency of a compound and represents the concentration where 50% of its maximal effect is observed.

The in vitro potency of the GLP-1 receptor agonists, such as derivatives, of the invention may be determined as described above, and the EC₅₀ of the GLP-1 receptor agonist (e.g. derivative) in question determined. The lower the EC₅₀ value, the better the potency. In an alternative or additional embodiment the GLP-1 receptor agonist (e.g. derivative) of the invention has an in vitro potency determined using the methods of Example C corresponding to an EC₅₀ at or below 10000 pM, more preferably below 5000 pM, even more preferably below 1000 pM, or most preferably below 500 pM.

In an alternative or additional embodiment the GLP-1 receptor agonists, such as derivatives, of the invention as well as the constituent GLP-1 peptides or analogues hereof as such are potent in vivo, which may be determined as is known in the art in any suitable animal model, as well as in clinical trials. The diabetic db/db mouse is one example of a suitable animal model, and the blood glucose lowering effect may be determined in such mice in vivo, e.g. as described in Example C, or as described in Example 43 of WO09/030738. The LYD pig is another example of a suitable animal model, and the reduction in food intake may be determined in a PD study in such pigs in vivo, e.g. as described in Example C. The GLP-1 receptor agonists, such as derivatives, of the invention are very potent in vivo, which is evidenced by a fine reduction in food intake in this PD study in pigs.

The GLP-1 receptor agonists, such as the derivatives, of the invention, as well as the constituent GLP-1 peptides or analogues hereof as such bind very well to the GLP-1 receptor at a low concentration of albumin. This may be determined as described in Example C. Generally, the binding to the GLP-1 receptor at low albumin concentration should be as good as possible, corresponding to a low IC₅₀ value.

In an alternative or additional embodiment the GLP-1 receptor binding affinity (IC₅₀) in the presence of 0.005% HSA (low albumin) is below 1000 nM, preferably below 600 nM, more preferably below 100 nM, or most preferably below 50 nM.

In an alternative or additional embodiment the GLP-1 receptor agonist, such as the derivative, of the invention is a full GLP-1 receptor agonist with a potency similar to GLP-1(7-37) (SEQ ID NO: 1). A full and potent GLP-1 receptor agonist may, e.g., be defined as a GLP-1 receptor agonist, such as a GLP-1 peptide or GLP-1 derivative, that (i) binds to the GLP-1 receptor with an IC₅₀ value equal to or less than 5 nM in a receptor binding affinity assay (such as the one of Example C herein); and (ii) activates the receptor with an EC₅₀ value equal to or less than 100 pM in a reporter gene assay (such as the one of Example C herein), and/or an EC₅₀ value equal to or less than 500 pM in a cAMP assay (such as the one of Example C herein).

The GLP-1 receptor agonists, such as the derivatives, of the invention may have improved pharmacokinetic properties such as increased terminal half-life, decreased clearance, and/or increased oral bioavailability. Increasing terminal half-life and/or decreasing of the clearance means that the compound in question is eliminated slower from the body. For the GLP-1 receptor agonists, such as derivatives, of the invention this entails an extended duration of pharmacological effect. Increased oral bioavailability means that a larger fraction of the dose administered orally reach the systemic circulation from where it can distribute to exhibit pharmacological effect.

The pharmacokinetic properties of the GLP-1 receptor agonists, such as derivatives, of the invention may suitably be determined in-vivo in pharmacokinetic (PK) studies. Such studies are conducted to evaluate how pharmaceutical compounds are absorbed, distributed, and eliminated in the body, and how these processes affect the concentration of the compound in the body, over the course of time.

In the discovery and preclinical phase of pharmaceutical drug development, animal models such as the mouse, rat, monkey, dog, or pig, may be used to perform this characterisation. Any of these models can be used to test the pharmacokinetic properties of the GLP-1 receptor agonists, such as derivatives, of the invention.

In such studies, animals are typically administered with a single dose of the drug, either intravenously, subcutaneously (s.c.), or orally (p.o.) in a relevant formulation. Blood samples are drawn at predefined time points after dosing, and samples are analysed for concentration of drug with a relevant quantitative assay. Based on these measurements, time-plasma concentration profiles for the compound of study are plotted and a so-called non-compartmental pharmacokinetic analysis of the data is performed.

For most compounds, the terminal part of the plasma-concentration profiles will be linear when drawn in a semi-logarithmic plot, reflecting that after the initial absorption and distribution, drug is removed from the body at a constant fractional rate. The rate (lambda Z or λ_{z}) is equal to minus the slope of the terminal part of the plot. From this rate, also a terminal half-life may be calculated, as t_{1/2}=ln(2)/λ₂ (see, e.g., Johan Gabrielsson and Daniel Weiner: Pharmacokinetics and Pharmacodynamic Data Analysis. Concepts & Applications, 3rd Ed., Swedish Pharmaceutical Press, Stockholm (2000)).

Clearance can be determined after i.v. administration and is defined as the dose (D) divided by area under the curve (AUC) on the plasma concentration versus time profile (Rowland, M and Tozer T N: Clinical Pharmacokinetics: Concepts and Applications, 3^{rd} edition, 1995 Williams Wilkins).

The estimate of terminal half-life and/or clearance is relevant for evaluation of dosing regimens and an important parameter in drug development, in the evaluation of new drug compounds.

The GLP-1 receptor agonists, such as the derivatives, of the invention may have improved pharmacokinetic properties.

In an alternative or additional embodiment the pharmacokinetic properties may be determined as terminal half-life (T_{1/2}) in vivo in rats after i.v. administration. In an alternative or additional embodiment the half-life is at least 4 hours, preferably at least 6 hours, even more preferably at least 8 hours, or most preferably at least 10 hours.

A suitable assay for determining terminal half-life in vivo in rats after i.v. administration is disclosed in Example C herein.

The GLP-1 receptor agonists, such as the derivatives, of the invention may have improved pharmacokinetic properties.

In an alternative or additional embodiment the pharmacokinetic properties may be determined as terminal half-life (T_{1/2}) in vivo in minipigs after i.v. administration, e.g. as described in Example C herein.

In an alternative or additional embodiment the terminal half-life in minipigs is at least 8 hours, preferably at least 24 hours, even more preferably at least 40 hours, or most preferably at least 60 hours.

The GLP-1 receptor agonist, such as the derivatives, of the invention may have a high oral bioavailability.

Generally, the term bioavailability refers to the fraction of an administered dose of the active pharmaceutical ingredient (API), such as a GLP-1 receptor agonist (e.g. derivative) of the invention that reaches the systemic circulation unchanged. By definition, when an API is administered intravenously, its bioavailability is 100%. However, when it is administered via other routes (such as orally), its bioavailability decreases (due to degradation and/or incomplete absorption and first-pass metabolism). Knowledge about bioavailability is important when calculating dosages for non-intravenous routes of administration.

A plasma concentration versus time plot is made after both oral and intravenous administration. The absolute bioavailability (F) is the (AUC-oral divided by dose), divided by (AUC-intravenous divided by dose). The GLP-1 receptor agonists, such as derivatives, of the invention have an absolute oral bioavailability which is higher than that of a) liraglutide, and/or b) semaglutide; preferably at least 10% higher, more preferably at least 20% higher, even more preferably at least 30% higher, or most preferably at least 40% higher.

Before testing oral bioavailability the GLP-1 receptor agonists, such as derivatives, of the invention may suitably be formulated as is known in the art of oral formulations of insulinotropic compounds, e.g. using any one or more of the formulations described in WO 2008/145728.

The GLP-1 receptor agonists, such as derivatives, of the invention may have good biophysical properties. These properties include but are not limited to physical stability and/or solubility. These and other biophysical properties may be measured using standard methods known in the art of protein chemistry. In an alternative or additional embodiment these properties are improved as compared to native GLP-1 (SEQ ID NO: 1). Changed oligomeric properties of the GLP-1 receptor agonists (e.g. derivatives) may be at least partly responsible for the improved biophysical properties.

### Methods of Preparation

The production of GLP-1 receptor agonists, such as peptides like GLP-1(7-37) and GLP-1 analogues, is well known in the art.

The GLP-1 moiety of the GLP-1 receptor agonists, such as derivatives, of the invention (or fragments thereof), may for instance be produced by classical peptide synthesis, e.g., solid phase peptide synthesis using t-Boc or Fmoc chemistry or other well established techniques, see, e.g., Greene and Wuts, “Protective Groups in Organic Synthesis”, John Wiley & Sons, 1999, Florencio Zaragoza Dorwald, “Organic Synthesis on solid Phase”, Wiley-VCH Verlag GmbH, 2000, and “Fmoc Solid Phase Peptide Synthesis”, Edited by W. C. Chan and P. D. White, Oxford University Press, 2000. In an alternative or additional embodiment the term “GLP-1 moiety” as used herein refers to the unbranched peptide part of the GLP-1 receptor agonist.

Also, or alternatively, the GLP-1 moiety of the GLP-1 receptor agonists, such as derivatives, of the invention (or fragments thereof) may be produced by recombinant methods, viz. by culturing a host cell containing a DNA sequence encoding the analogue and capable of expressing the peptide in a suitable nutrient medium under conditions permitting the expression of the peptide. Non-limiting examples of host cells suitable for expression of these peptides are: Escherichia coli, Saccharomyces cerevisiae, as well as mammalian BHK or CHO cell lines.

Those GLP-1 receptor agonists, such as derivatives, of the invention which include non-natural amino acids and/or a covalently attached N-terminal mono- or dipeptide mimetic may e.g. be produced as described in the experimental part. Or see e.g., Hodgson et al: “The synthesis of peptides and proteins containing non-natural amino acids”, Chemical Society Reviews, vol. 33, no. 7 (2004), p. 422-430; and WO 2009/083549 A1 entitled “Semi-recombinant preparation of GLP-1 analogues”.

Specific examples of methods of preparing a number of the GLP-1 receptor agonists, such as derivatives, of the invention are mentioned herein.

### Pharmaceutical Compositions

In an alternative or additional embodiment the GLP-1 receptor agonist is formulated together with a pharmaceutically acceptable carrier, vehicle, diluent and/or excipient. Such formulation may be referred to as a pharmaceutical composition herein. Pharmaceutical compositions comprising a GLP-1 receptor agonist, such as a derivative, of the invention or a pharmaceutically acceptable salt, amide, or ester thereof, and a pharmaceutically acceptable excipient may be prepared as is known in the art. The term “excipient” broadly refers to any component other than the active therapeutic ingredient(s). The excipient may be an inert substance, an inactive substance, and/or a not medicinally active substance. The excipient may serve various purposes, e.g. as a carrier, vehicle, diluent, tablet aid, and/or to improve administration, and/or absorption of the active substance. The formulation of pharmaceutically active ingredients with various excipients is known in the art, see e.g. Remington: The Science and Practice of Pharmacy (e.g. 19^{th} edition (1995), and any later editions). Non-limiting examples of excipients are: Solvents, diluents, buffers, preservatives, tonicity regulating agents, chelating agents, and stabilisers.

Examples of formulations include liquid formulations, i.e. aqueous formulations comprising water. A liquid formulation may be a solution, or a suspension. An aqueous formulation typically comprises at least 50% w/w water, or at least 60%, 70%, 80%, or even at least 90% w/w of water. Alternatively, a pharmaceutical composition may be a solid formulation, e.g. a freeze-dried or spray-dried composition, which may be used as is, or whereto the physician or the patient adds solvents, and/or diluents prior to use. The pH in an aqueous formulation may be anything between pH 3 and pH 10, for example from about 7.0 to about 9.5; or from about 3.0 to about 7.0.

A pharmaceutical composition may comprise a buffer. The buffer may e.g. be selected from sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid, and mixtures thereof.

A pharmaceutical composition may comprise a preservative. The preservative may e.g. be selected from phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol), and mixtures thereof. The preservative may be present in a concentration from 0.1 mg/ml to 20 mg/ml. A pharmaceutical composition may comprise an isotonic agent. The isotonic agent may e.g. be selected from a salt (e.g. sodium chloride), a sugar or sugar alcohol, an amino acid (e.g. glycine, histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol)polyethyleneglycol (e.g. PEG400), and mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, alfa and beta HPCD, soluble starch, hydroxyethyl starch and carboxymethylcellulose-Na may be used. Sugar alcohol is defined as a C4-C8 hydrocarbon having at least one —OH group and includes, for example, mannitol, sorbitol, inositol, galactitol, dulcitol, xylitol, and arabitol. In an alternative or additional embodiment, the sugar alcohol additive is mannitol.

A pharmaceutical composition may comprise a chelating agent. The chelating agent may e.g. be selected from salts of ethylenediaminetetraacetic acid (EDTA), citric acid, and aspartic acid, and mixtures thereof.

A pharmaceutical composition may comprise a stabiliser. The stabiliser may e.g. be one or more oxidation inhibitors, aggregation inhibitors, surfactants, and/or one or more protease inhibitors. Non-limiting examples of these various kinds of stabilisers are disclosed in the following.

The term “aggregate formation” refers to a physical interaction between the polypeptide molecules resulting in formation of oligomers, which may remain soluble, or large visible aggregates that precipitate from the solution. Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

A pharmaceutical composition may comprise an amount of an amino acid base sufficient to decrease aggregate formation of the polypeptide during storage of the composition. The term “amino acid base” refers to one or more amino acids (such as methionine, histidine, imidazole, arginine, lysine, isoleucine, aspartic acid, tryptophan, threonine), or analogues thereof. Any amino acid may be present either in its free base form or in its salt form. Any stereoisomer (i.e., L, D, or a mixture thereof) of the amino acid base may be present.

Methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. Any stereoisomer of methionine (L or D) or combinations thereof can be used.

A pharmaceutical composition may comprise a stabiliser selected from high molecular weight polymers or low molecular compounds. The stabiliser may e.g. be selected from polyethylene glycol (e.g. PEG 3350), polyvinyl alcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). A pharmaceutical composition may comprise additional stabilising agents such as, but not limited to, methionine and EDTA, which protect the polypeptide against methionine oxidation, and a nonionic surfactant, which protects the polypeptide against aggregation associated with freeze-thawing or mechanical shearing.

A pharmaceutical composition may comprise one or more surfactants. The term “surfactant” refers to any molecules or ions that are comprised of a water-soluble (hydrophilic) part, and a fat-soluble (lipophilic) part. The surfactant may e.g. be selected from anionic surfactants, cationic surfactants, nonionic surfactants, and/or zwitterionic surfactants.

A pharmaceutical composition may comprise one or more protease inhibitors, such as, e.g., EDTA (ethylenediamine tetraacetic acid), and/or benzamidine HCl.

Additional, optional, ingredients of a pharmaceutical composition include, e.g., wetting agents, emulsifiers, antioxidants, bulking agents, metal ions, oily vehicles, proteins (e.g., human serum albumin, gelatine), and/or a zwitterion (e.g., an amino acid such as betaine, taurine, arginine, glycine, lysine and histidine).

Still further, a pharmaceutical composition may be formulated as is known in the art of oral formulations of insulinotropic compounds, e.g. using any one or more of the formulations described in WO 2008/145728.

A composition may be administered in several dosage forms, for example as a solution; a suspension; an emulsion; a microemulsion; multiple emulsions; a foam; a salve; a paste; a plaster; an ointment; a tablet; a coated tablet; a chewing gum; a rinse; a capsule such as hard or soft gelatine capsules; a suppositorium; a rectal capsule; drops; a gel; a spray; a powder; an aerosol; an inhalant; eye drops; an ophthalmic ointment; an ophthalmic rinse; a vaginal pessary; a vaginal ring; a vaginal ointment; an injection solution; an in situ transforming solution such as in situ gelling, setting, precipitating, and in situ crystallisation; an infusion solution; or as an implant. A composition may be a tablet, optionally coated, a capsule, or a chewing gum. A composition may further be compounded in a drug carrier or drug delivery system, e.g. in order to improve stability, bioavailability, and/or solubility. In an alternative or additional embodiment a composition may be attached to such system through covalent, hydrophobic, and/or electrostatic interactions. The purpose of such compounding may be, e.g., to decrease adverse effects, achieve chronotherapy, and/or increase patient compliance.

A composition may also be used in the formulation of controlled, sustained, protracting, retarded, and/or slow release drug delivery systems. Parenteral administration may be performed by subcutaneous, intramuscular, intraperitoneal, or intravenous injection by means of a syringe, optionally a pen-like syringe, or by means of an infusion pump. A composition may be administered nasally in the form of a solution, a suspension, or a powder; or it may be administered pulmonally in the form of a liquid or powder spray. Transdermal administration is a still further option, e.g. by needle-free injection, from a patch such as an iontophoretic patch, or via a transmucosal route, e.g. buccally. A composition may be a stabilised formulation. The term “stabilised formulation” refers to a formulation with increased physical and/or chemical stability, preferably both. In general, a formulation must be stable during use and storage (in compliance with recommended use and storage conditions) until the expiration date is reached.

The term “physical stability” refers to the tendency of the polypeptide to form biologically inactive and/or insoluble aggregates as a result of exposure to thermo-mechanical stress, and/or interaction with destabilising interfaces and surfaces (such as hydrophobic surfaces). The physical stability of an aqueous polypeptide formulation may be evaluated by means of visual inspection, and/or by turbidity measurements after exposure to mechanical/physical stress (e.g. agitation) at different temperatures for various time periods. Alternatively, the physical stability may be evaluated using a spectroscopic agent or probe of the conformational status of the polypeptide such as e.g.

Thioflavin T or “hydrophobic patch” probes.

The term “chemical stability” refers to chemical (in particular covalent) changes in the polypeptide structure leading to formation of chemical degradation products potentially having a reduced biological potency, and/or increased immunogenic effect as compared to the intact polypeptide. The chemical stability can be evaluated by measuring the amount of chemical degradation products at various time-points after exposure to different environmental conditions, e.g. by SEC-HPLC, and/or RP-HPLC.

### Administration

In an alternative or additional embodiment the dose of GLP-1 receptor agonist is 0.6 mg/day. In an alternative or additional embodiment the dose of GLP-1 receptor agonist is 1.2 mg/day. In an alternative or additional embodiment the dose of GLP-1 receptor agonist is 1.8 mg/day.

In an alternative or additional embodiment the GLP-1 receptor agonist is for use in treatment period of greater than one day (for example, a period of, or at least of, 2 days, a period of, or at least of, 3 days, a period of, or at least of, 4 days, a period of, or at least of, 5 days, a period of, or at least of, 6 days, a period of, or at least of, 1 week, a period of, or at least of, 2 weeks, a period of, or at least of, 3 weeks, a period of, or at least of, 4 weeks, a period of, or at least of, 1 month, a period of, or at least of, 2 months, a period of, or at least of, 3 months, a period of, or at least of, 4 months, a period of, or at least of, 5 months, a period of, or at least of, 6 months, and a continuous or indefinite period). The GLP-1 receptor agonist may be for use in a daily treatment. The GLP-1 receptor agonist may be for use in a weekly treatment. In an alternative or additional embodiment the GLP-1 receptor agonist is for use in treatment period of greater than one day. In an alternative or additional embodiment the GLP-1 receptor agonist is for use in treatment period of, or at least of, at least of, 2 days. In an alternative or additional embodiment the GLP-1 receptor agonist is for use in treatment period of, or at least of, 3 days. In an alternative or additional embodiment the GLP-1 receptor agonist is for use in treatment period of, or at least of, 4 days. In an alternative or additional embodiment the GLP-1 receptor agonist is for use in treatment period of, or at least of, 5 days. In an alternative or additional embodiment the GLP-1 receptor agonist is for use in treatment period of, or at least of, 6 days. In an alternative or additional embodiment the GLP-1 receptor agonist is for use in treatment period of, or at least of, 1 week. In an alternative or additional embodiment the GLP-1 receptor agonist is for use in treatment period of, or at least of, 2 weeks. In an alternative or additional embodiment the GLP-1 receptor agonist is for use in treatment period of, or at least of, 3 weeks. In an alternative or additional embodiment the GLP-1 receptor agonist is for use in treatment period of, or at least of, 4 weeks. In an alternative or additional embodiment the GLP-1 receptor agonist is for use in treatment period of, or at least of, 1 month. In an alternative or additional embodiment the GLP-1 receptor agonist is for use in treatment period of, or at least of, 2 months. In an alternative or additional embodiment the GLP-1 receptor agonist is for use in treatment period of, or at least of, 3 months. In an alternative or additional embodiment the GLP-1 receptor agonist is for use in treatment period of, or at least of, 4 months. In an alternative or additional embodiment the GLP-1 receptor agonist is for use in treatment period of, or at least of, 5 months. In an alternative or additional embodiment the GLP-1 receptor agonist is for use in treatment period of, or at least of, 6 months. In an alternative or additional embodiment the GLP-1 receptor agonist is for use in a continuous or indefinite treatment period). In an alternative or additional embodiment the GLP-1 receptor agonist is for use in daily treatment. In an alternative or additional embodiment the GLP-1 receptor agonist is for use in weekly treatment. By “treatment period” we mean the period over which a therapeutically effective dose of GLP-1 receptor agonist is provided to an individual in need of treatment. In an alternative or additional embodiment the term “daily treatment” refers to a treatment regimen with one administration of GLP-1 receptor agonist per day. In an alternative embodiment, by “daily treatment” a treatment regimen with one administration of GLP-1 per day. In an alternative or additional embodiment the term “weekly treatment” refers to a treatment regimen with one administration of GLP-1 receptor agonist per week. In an alternative embodiment, by “weekly treatment” a treatment regimen with one administration of GLP-1 per week.

An administered dose may contain from 0.01 mg-100 mg of the GLP-1 receptor agonist (e.g. derivative), or from 0.01-50 mg, or from 0.01-20 mg, or from 0.01-10 mg of the GLP-1 receptor agonist (e.g. derivative).

The GLP-1 receptor agonist (e.g. derivative) may be administered in the form of a pharmaceutical composition. It may be administered to a patient in need thereof at several sites, for example, at topical sites such as skin or mucosal sites; at sites which bypass absorption such as in an artery, in a vein, or in the heart; and at sites which involve absorption, such as in the skin, under the skin, in a muscle, or in the abdomen.

The route of administration may be, for example, lingual; sublingual; buccal; in the mouth; oral; in the stomach; in the intestine; nasal; pulmonary, such as through the bronchioles, the alveoli, or a combination thereof; parenteral, epidermal; dermal; transdermal; conjunctival; uretal; vaginal; rectal; and/or ocular. A composition may be an oral composition, and the route of administration is per oral.

The treatment with a GLP-1 receptor agonist, such as a derivative, according to the present invention may also be combined with one or more additional pharmacologically active substances, e.g. selected from antidiabetic agents, antiobesity agents, appetite regulating agents, antihypertensive agents, agents for the treatment and/or prevention of complications resulting from or associated with diabetes and agents for the treatment and/or prevention of complications and disorders resulting from or associated with obesity. Examples of these pharmacologically active substances are: Insulin, sulphonylureas, biguanides, meglitinides, glucosidase inhibitors, glucagon antagonists, DPP-IV (dipeptidyl peptidase-IV) inhibitors, inhibitors of hepatic enzymes involved in stimulation of gluconeogenesis and/or glycogenolysis, glucose uptake modulators, compounds modifying the lipid metabolism such as antihyperlipidemic agents as HMG CoA inhibitors (statins), Gastric Inhibitory Polypeptides (GIP analogs), compounds lowering food intake, RXR agonists and agents acting on the ATP-dependent potassium channel of the β-cells; Cholestyramine, colestipol, clofibrate, gemfibrozil, lovastatin, pravastatin, simvastatin, probucol, dextrothyroxine, neteglinide, repaglinide; β-blockers such as alprenolol, atenolol, timolol, pindolol, propranolol and metoprolol, ACE (angiotensin converting enzyme) inhibitors such as benazepril, captopril, enalapril, fosinopril, lisinopril, alatriopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin; CART (cocaine amphetamine regulated transcript) agonists, NPY (neuropeptide Y) antagonists, PYY agonists, Y2 receptor agonists, Y4 receptor agonits, mixed Y2/Y4 receptor agonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 agonists, oxyntomodulin and analogues, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, RXR (retinoid X receptor) modulators, TR β agonists; histamine H3 antagonists, Gastric Inhibitory Polypeptide agonists or antagonists (GIP analogs), gastrin and gastrin analogs.

In an alternative or additional embodiment the one or more further or additional drug is administered, concurrently or consecutively, with the GLP-1 receptor agonist.

In an alternative or additional embodiment the GLP-1 receptor agonist is for use in combination with one or more additional anti-diabetic drug.

By “additional diabetic drug” we include any agent used in the treatment of diabetes other than the GLP-1 receptor agonist (or the GLP-1 receptor agonist for use). Preferably, the “additional diabetic drug” is not a GLP-1 receptor agonist.

In an alternative or additional embodiment the one or more further or additional drug is administered, concurrently or consecutively, with the GLP-1 receptor agonist.

In an alternative or additional embodiment the one or more additional anti-diabetic drug is administered, concurrently or consecutively, with the GLP-1 receptor agonist.

In an alternative or additional embodiment the one or more additional anti-diabetic drug is administered, concurrently with the GLP-1 receptor agonist for use in the same formulation.

In an alternative or additional embodiment the one or more additional anti-diabetic drug is selected from the group consisting of: Insulins; and Sensitizers (such as biguanides and thiazolidinediones).

In an alternative or additional embodiment the one or more anti-diabetic drug is, or includes, an insulin. An “insulin” or “naturally occurring insulin” according to the invention is herein to be understood as human insulin or an insulin from another species such as porcine or bovine insulin. The term “insulin peptide” as used herein means a peptide which is either human insulin or an analog or a derivative thereof with insulin activity. The term “parent insulin” as used herein is intended to mean an insulin before any modifications have been applied thereto. The term “insulin analogue” as used herein means a modified human insulin wherein one or more amino acid residues of the insulin have been substituted by other amino acid residues and/or wherein one or more amino acid residues have been deleted from the insulin and/or wherein one or more amino acid residues have been added and/or inserted to the insulin.

In an alternative or additional embodiment an insulin analogue comprises 10 or fewer amino acid modifications (substitutions, deletions, additions (including insertions) and any combination thereof) relative to human insulin. In an alternative or additional embodiment an insulin analogue comprises 9 or fewer amino acid modifications relative to human insulin. In an alternative or additional embodiment an insulin analogue comprises 8 or fewer amino acid modifications relative to human insulin. In an alternative or additional embodiment an insulin analogue comprises 7 or fewer amino acid modifications relative to human insulin. In an alternative or additional embodiment an insulin analogue comprises 6 or fewer amino acid modifications relative to human insulin. In an alternative or additional embodiment an insulin analogue comprises 5 or fewer amino acid modifications relative to human insulin. In an alternative or additional embodiment an insulin analogue comprises 4 or fewer amino acid modifications relative to human insulin. In an alternative or additional embodiment an insulin analogue comprises 3 or fewer amino acid modifications relative to human insulin. In an alternative or additional embodiment an insulin analogue comprises 2 or fewer amino acid modifications relative to human insulin. In an alternative or additional embodiment an insulin analogue comprises 1 modification relative to human insulin.

Modifications in the insulin molecule are denoted stating the chain (A or B), the position, and the one or three letter code for the amino acid residue substituting the native amino acid residue.

By “desB30” or “B(1-29)” is meant a natural insulin B chain or an analogue thereof lacking the B30 amino acid and “A(1-21)” means the natural insulin A chain. Thus, e.g., A21Gly, B28Asp, desB30 human insulin is an analogue of human insulin where the amino acid in position 21 in the A chain is substituted with glycine, the amino acid in position 28 in the B chain is substituted with aspartic acid, and the amino acid in position 30 in the B chain is deleted.

Herein terms like “A1”, “A2” and “A3” etc. indicates the amino acid in position 1, 2 and 3 etc., respectively, in the A chain of insulin (counted from the N-terminal end). Similarly, terms like B1, B2 and B3 etc. indicates the amino acid in position 1, 2 and 3 etc., respectively, in the B chain of insulin (counted from the N-terminal end). Using the one letter codes for amino acids, terms like A21A, A21G and A21Q designates that the amino acid in the A21 position is A, G and Q, respectively. Using the three letter codes for amino acids, the corresponding expressions are A21Ala, A21Gly and A21Gln, respectively.

Herein the terms “A(0)” or “B(0)” indicate the positions of the amino acids N-terminally to A1 or B1, respectively. The terms A(−1) or B(−1) indicate the positions of the first amino acids N-terminally to A(0) or B(0), respectively. Thus A(−2) and B(−2) indicate positions of the amino acids N-terminally to A(−1) and B(−1), respectively, A(−3) and B(−3) indicate positions of the amino acids N-terminally to A(−2) and B(−2), respectively, and so forth. The terms A22 or B31 indicate the positions of the amino acids C-terminally to A21 or B30, respectively. The terms A23 or B32 indicate the positions of the first amino acids C-terminally to A22 or B31, respectively. Thus A24 and B33 indicate positions of the amino acids C-terminally to A23 and B32, respectively, and so forth.

Herein, the term “amino acid residue” is an amino acid from which, formally, a hydroxy group has been removed from a carboxy group and/or from which, formally, a hydrogen atom has been removed from an amino group.

Examples of insulin analogues are such wherein Pro in position 28 of the B chain is substituted with Asp, Lys, Leu, Val, or Ala and/or Lys at position B29 is substituted with Pro, Glu or Asp. Furthermore, Asn at position B3 may be substituted with Thr, Lys, Gln, Glu or Asp. The amino acid residue in position A21 may be substituted with Gly. Also one or more amino acids may be added to the C-terminal of the A-chain and/or B-chain such as, e.g., Lys. The amino acid in position B1 may be substituted with Glu. The amino acid in position B16 may be substituted with Glu or His. Further examples of insulin analogues are the deletion analogues, e.g., analogues where the B30 amino acid in human insulin has been deleted (des(B30) human insulin), insulin analogues wherein the B1 amino acid in human insulin has been deleted (des(B1) human insulin), des(B28-B30) human insulin and des(B27) human insulin. Insulin analogues wherein the A-chain and/or the B-chain have an N-terminal extension and insulin analogues wherein the A-chain and/or the B-chain have a C-terminal extension such as with two arginine residues added to the C-terminal of the B-chain are also examples of insulin analogues. Further examples are insulin analogues comprising combinations of the mentioned mutations. Insulin analogues wherein the amino acid in position A14 is Asn, Gln, Glu, Arg, Asp, Gly or His, the amino acid in position B25 is His and which optionally further comprises one or more additional mutations are further examples of insulin analogues. Insulin analogues of human insulin wherein the amino acid residue in position A21 is Gly and wherein the insulin analogue is further extended in the C-terminal with two arginine residues are also examples of insulin analogues.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### Non-Limiting List of Embodiments

1. A GLP-1 receptor agonist for use in treating type 1 diabetes, wherein said use comprises reducing the number of hypoglycaemias or hypoglycaemic episodes.
2. A GLP-1 receptor agonist for use in treating type 1 diabetes, wherein said use comprises reducing the severity of blood glucose excursions.
3. A GLP-1 receptor agonist for use in treating type 1 diabetes, wherein said use comprises reducing the amount of exogenous glucose needed for recovering from a hypoglycaemia or a hypoglycaemic episode.
4. A GLP-1 receptor agonist for use in treating type 1 diabetes, wherein said use comprises reducing the duration of hypoglycaemia or hypoglycaemic episodes.
5. A GLP-1 receptor agonist for use in treating or preventing hypoglycaemia or hypoglycaemic episodes in patients suffering from type 1 diabetes.
6. The GLP-1 receptor agonist for use according to any one of the preceding embodiments wherein said reduction as defined in any one of the preceding embodiments as compared to the subject, to whom the GLP-1 receptor agonist is administered, prior to treatment with a GLP-1 receptor agonist.
7. The GLP-1 receptor agonist for use according to any one of the preceding embodiments wherein said reduction as defined in any one of the preceding embodiments is as compared to placebo not comprising the GLP-1 receptor agonist.
8. The GLP-1 receptor agonist for use according to any one of the preceding embodiments wherein said reduction as defined in any one of the preceding embodiments is at least 5%, such as at least 10% or at least 15%.
9. The GLP-1 receptor agonist for use according to any one of the preceding embodiments wherein said number of hypoglycaemias or hypoglycaemic episodes suffered by a subject over a period of time is reduced compared to the number of episodes suffered by said subject for the same period of time prior to administration of said GLP-1 receptor agonist.
10. The GLP-1 receptor agonist for use according to any one of the preceding embodiments wherein the period of time is one year.
11. The GLP-1 receptor agonist for use according to any one of the previous embodiments wherein the GLP-1 receptor agonist is suitable for once daily administration.
12. The GLP-1 receptor agonist for use according to any one of the preceding embodiments wherein the GLP-1 receptor agonist is suitable for once weekly administration.
13. The GLP-1 receptor agonist for use according to any one of the preceding embodiments wherein the GLP-1 receptor agonist is a GLP-1 fragment, derivative or analogue.
14. The GLP-1 receptor agonist for use according to any one of the preceding embodiments wherein the GLP-1 receptor agonist comprises no more than 5, such as no more than 4 or no more than 3, amino acid residues which have been substituted, inserted or deleted as compared to GLP-1(7-37).
15. The GLP-1 receptor agonist for use according to any one of the preceding embodiments wherein the GLP-1 derivative or analogue is selected from the group consisting of: GLP-1(7-37); GLP-1(7-36) amide; Exenatide; Exenatide LAR; Liraglutide; Semaglutide; Taspoglutide; Albiglutide; Lixisenatide; and Dulaglutide.
16. The GLP-1 receptor agonist for use according to any one of the preceding embodiments wherein the GLP-1 receptor agonist is selected from the group consisting of Liraglutide and Semaglutide.
17. The GLP-1 receptor agonist for use according to any one of the preceding embodiments wherein GLP-1 receptor agonist is a long-acting GLP-1 derivative or analogue.
18. The GLP-1 receptor agonist for use according to any one of the preceding embodiments wherein the long-acting GLP-1 derivative or analogue is selected from the group consisting of: Liraglutide; Semaglutide; Taspoglutide; Albiglutide; Taspoglutide; Dulaglutide; and Exenatide LAR.
19. The GLP-1 receptor agonist for use according to any one of the preceding embodiments wherein the long-acting GLP-1 derivative or analogue is Liraglutide.
20. The GLP-1 receptor agonist for use according to any one of the preceding embodiments wherein the long-acting GLP-1 derivative or analogue is Semaglutide.
21. The GLP-1 receptor agonist according to any one of the preceding embodiments for use in treating hypoglycaemia or hypoglycaemic episodes associated with insulin therapy of type 1 diabetes.
22. The GLP-1 receptor agonist for use according to any one of the preceding embodiments wherein the GLP-1 receptor agonist is for use in the treatment or prevention of one or more additional symptoms associated with insulin therapy of type 1 diabetes.
23. The GLP-1 receptor agonist for use according to any one of the preceding embodiments, wherein the one or more additional symptoms associated with insulin therapy of type 1 diabetes are selected from the group consisting of: reduced glycaemic control; insulin deficiency; and obesity.
24. The GLP-1 receptor agonist for use according to any one of the preceding embodiments wherein the treatment or prevention of hypoglycaemia or hypoglycaemic episodes in patients suffering from type 1 diabetes comprises or consists of treating or preventing one or more symptom selected from the group consisting of: hypoglycaemia; overall hypoglycaemic episodes; nocturnal hypoglycaemic episodes; and daytime hypoglycaemic episodes.
25. The GLP-1 receptor agonist for use according to any one of the preceding embodiments wherein the GLP-1 receptor agonist is capable of reducing overall nocturnal hypoglycaemic episodes.
26. The GLP-1 receptor agonist for use according to any one of the preceding embodiments wherein the GLP-1 receptor agonist is capable of reducing the duration of nocturnal hypoglycaemic episodes.
27. The GLP-1 receptor agonist for use according to any one of the preceding embodiments wherein the GLP-1 receptor agonist is capable of reducing overall nocturnal hypoglycaemic episodes by at least 25%, for example, 30%, 35%, or 40%.
28. The GLP-1 receptor agonist for use according to any one of the preceding embodiments wherein the GLP-1 receptor agonist is capable of reducing overall nocturnal hypoglycaemic episodes by at least 31%.
29. The GLP-1 receptor agonist for use according to any one of the preceding embodiments wherein the GLP-1 receptor agonist is capable of reducing overall nocturnal hypoglycaemic episodes by at least 35%.
30. The GLP-1 receptor agonist for use according to any one of the preceding embodiments wherein the GLP-1 receptor agonist is capable of reducing overall nocturnal hypoglycaemic episodes by at least 39%.
31. The GLP-1 receptor agonist for use according to any one of the preceding embodiments wherein the dose of GLP-1 receptor agonist is 0.6 mg/day.
32. The GLP-1 receptor agonist for use any one of the preceding embodiments wherein the dose of GLP-1 receptor agonist is 1.2 mg/day.
33. The GLP-1 receptor agonist for use according to any one of the preceding embodiments wherein the dose of GLP-1 receptor agonist is 1.2 mg/day.
34. The GLP-1 receptor agonist for use according to any of the preceding embodiments wherein the GLP-1 receptor agonist is capable of reducing the amount of exogenous glucose needed for recovering from a hypoglycaemic episode and/or reducing the severity of blood glucose excursions.
35. The GLP-1 receptor agonist for use according to any of the preceding embodiments wherein the GLP-1 receptor agonist, when administered at a dose 1.2 mg once daily, is capable of reducing the amount of exogenous glucose needed for recovering after a hypoglycaemic episode by at least 19%.
36. The GLP-1 receptor agonist for use according to any according to any one of the preceding embodiments wherein the GLP-1 receptor agonist, when administered in a dose 0.6 mg once daily, is capable of reducing the amount of exogenous glucose needed for recovering after a hypoglycaemic episode by at least 23%.
37. The GLP-1 receptor agonist for use according to any according to any one of the preceding embodiments wherein the GLP-1 receptor agonist, when administered in a dose 1.8 mg once daily, is capable of reducing the amount of exogenous glucose needed for recovering after a hypoglycaemic episode by at least about 37% relative to placebo.
38. The GLP-1 receptor agonist for use according to any one of the preceding embodiments wherein the GLP-1 receptor agonist is administered at a dose sufficient to reduce the exogenous glucose requirement of the patient to recover from a hypoglycaemic episode.
39. The GLP-1 receptor agonist for use according to any one of the preceding embodiments wherein the GLP-1 receptor agonist is administered at a dose sufficient to maximally reduce the exogenous glucose requirement in the patient to recover from a potential hypoglycaemic episode.
40. The GLP-1 receptor agonist for use according to any one of the preceding embodiments wherein the GLP-1 receptor agonist is semaglutide.
41. The GLP-1 receptor agonist for use according to any one of the preceding embodiments wherein the GLP-1 receptor agonist administered is liraglutide and the dose is sufficient to optimally reduce the exogenous glucose requirement in the patient recovering from potential hypoglycaemic episodes is between 0.6 mg per day and 1.8 mg per day (for example, between 0.6 mg and 1.2 mg per day).
42. The GLP-1 receptor agonist for use according to any one of the preceding embodiments wherein the GLP-1 receptor agonist is liraglutide and the dose sufficient to optimally reduce the exogenous glucose requirement in the patient recovering from potential hypoglycaemic episodes is selected from the group consisting of 0.6 mg per day; 1.2 mg per day and 1.8 mg per day.
43. The GLP-1 receptor agonist for use according to any one of the preceding embodiments wherein the GLP-1 receptor agonist is administered at a dose of 0.6 mg once daily to reduce the exogenous glucose requirement of the patient to recover from a hypoglycaemic episode.
44. The GLP-1 receptor agonist for use according to any one of the preceding embodiments wherein the GLP-1 receptor agonist is administered at a dose of 1.2 mg once daily to reduce the exogenous glucose requirement of the patient to recover from a hypoglycaemic episode.
45. The GLP-1 receptor agonist for use according to any one of the preceding embodiments wherein the GLP-1 receptor agonist is administered at a dose of 1.8 mg once daily to reduce the exogenous glucose requirement of the patient to recover from a hypoglycaemic episode.
46. The GLP-1 receptor agonist for use according to any one of the preceding embodiments wherein the type 1 diabetes exhibits complete or partial insulin deficiency.
47. The GLP-1 receptor agonist for use according to any one of the preceding embodiments wherein the type 1 diabetes exhibits absolute, or substantially absolute, insulin deficiency.
48. The GLP-1 receptor agonist for use according to any one of the preceding embodiments wherein the GLP-1 receptor agonist is formulated together with a pharmaceutically acceptable carrier, vehicle, diluent and/or excipient.
49. The GLP-1 receptor agonist for use according to any one of the preceding embodiments wherein the GLP-1 receptor agonist is for use in treatment period of greater than one day (for example, a period of, or at least of, 2 days, a period of, or at least of, 3 days, a period of, or at least of, 4 days, a period of, or at least of, 5 days, a period of, or at least of, 6 days, a period of, or at least of, 1 week, a period of, or at least of, 2 weeks, a period of, or at least of, 3 weeks, a period of, or at least of, 4 weeks, a period of, or at least of, 1 month, a period of, or at least of, 2 months, a period of, or at least of, 3 months, a period of, or at least of, 4 months, a period of, or at least of, 5 months, a period of, or at least of, 6 months, and a continuous or indefinite period).
50. The GLP-1 receptor agonist for use according to any one of the preceding embodiments wherein the GLP-1 receptor agonist is for use in a daily treatment.
51. The GLP-1 receptor agonist for use according to any one of the preceding embodiments wherein the GLP-1 receptor agonist is for use in a weekly treatment.
52. The GLP-1 receptor agonist for use according to any one of the preceding embodiments wherein the GLP-1 receptor agonist is for use in combination with one or more additional drug.
53. The GLP-1 receptor agonist for use according to any one of the preceding embodiments wherein the one or more further or additional drug is administered, concurrently or consecutively, with the GLP-1 receptor agonist.
54. The GLP-1 receptor agonist for use according to any one of the preceding embodiments wherein the GLP-1 receptor agonist is for use in combination with one or more additional anti-diabetic drug.
55. The GLP-1 receptor agonist for use according to any one of the preceding embodiments wherein the one or more additional anti-diabetic drug is administered, concurrently or consecutively, with the GLP-1 receptor agonist.
56. The GLP-1 receptor agonist for use according to any one of the preceding embodiments wherein the one or more additional anti-diabetic drug is administered, concurrently with the GLP-1 receptor agonist for use in the same formulation.
57. The GLP-1 receptor agonist for use according to any one according to any one of the preceding embodiments wherein the one or more additional anti-diabetic drug is selected from the group consisting of: Insulins; and Sensitizers (such as biguanides and thiazolidinediones).
58. The GLP-1 receptor agonist for use according to any one of the preceding embodiments wherein the one or more anti-diabetic drug is, or includes, an insulin.
59. The GLP-1 receptor agonist for use according to any one of the preceding embodiments wherein the use and/or the GLP-1 receptor agonist is as defined herein.
60. The use of a GLP-1 agonist in the preparation of a medicament for
   i) reducing the number of hypoglycaemic episodes;
   ii) reducing the severity of blood glucose excursions;
   iii) reducing the amount of exogenous glucose needed for recovering from a hypoglycaemic episode;
   iv) reducing the duration of hypoglycaemic episodes; and/or
   v) treating or preventing hypoglycaemia or hypoglycaemic episodes;
in patients suffering from type 1 diabetes.
61. The use of a GLP-1 receptor agonist in the preparation of a medicament according to the preceding embodiment wherein the use and/or the GLP-1 receptor agonist is as defined herein.
62. A method for reducing the number of hypoglycaemias or hypoglycaemic episodes suffered by a subject being treated with insulin, said method comprising administering to said subject an amount of a GLP-1 receptor agonist effective to reduce the number of hypoglycaemias or hypoglycaemic episodes suffered by said subject.
63. A method for reducing the severity of blood glucose excursions suffered by a subject being treated with insulin, said method comprising administering to said subject an amount of a GLP-1 receptor agonist effective to reduce the severity of blood glucose excursions suffered by said subject.
64. A method for reducing the amount of exogenous glucose needed for recovering from a hypoglycaemia or a hypoglycaemic episode in a subject being treated with insulin, said method comprising administering to said subject an amount of a GLP-1 receptor agonist effective to reduce the amount of exogenous glucose needed for recovering from a hypoglycaemia or a hypoglycaemic episode by said subject.
65. A method for reducing the duration of hypoglycaemias or hypoglycaemic episodes in a subject being treated with insulin, said method comprising administering to said subject an amount of a GLP-1 receptor agonist effective to reduce the duration of hypoglycaemias or hypoglycaemic episodes suffered by said subject.
66. The method according to any one of the preceding embodiments wherein the subject has type 1 diabetes.
67. A method for treating type 1 diabetes in a subject in need of such treatment, said method comprising administering to said subject an amount of a GLP-1 receptor agonist effective to treat type 1 diabetes and reduce the number of hypoglycaemias or hypoglycaemic episodes suffered by said subject.
68. A method for treating type 1 diabetes in a subject in need of such treatment, said method comprising administering to said subject an amount of a GLP-1 receptor agonist effective to treat type 1 diabetes and reduce the severity of blood glucose excursions suffered by said subject.
69. A method for treating type 1 diabetes in a subject in need of such treatment, said method comprising administering to said subject an amount of a GLP-1 receptor agonist effective to treat type 1 diabetes and reduce the amount of exogenous glucose needed for recovering from a hypoglycaemia or a hypoglycaemic episode suffered by said subject.
70. A method for treating type 1 diabetes in a subject in need of such treatment, said method comprising administering to said subject an amount of a GLP-1 receptor agonist effective to treat type 1 diabetes and reduce the duration of hypoglycaemias or hypoglycaemic episodes suffered by said subject.
71. The method according to embodiment 62 or 66 wherein said number of hypoglycaemias or hypoglycaemic episodes suffered by said subject in a period of time is reduced compared to the number of hypoglycaemias or hypoglycaemic episodes suffered by said subject for the same period of time prior to administration of said GLP-1 receptor agonist.
72. The method according to embodiment 70 wherein the period of time is one year.
73. The method according to any one of the preceding embodiments wherein said reduction is as compared to the subject prior to treatment with a GLP-1 receptor agonist.
74. The method according to any one of the preceding embodiments wherein said reduction is as compared to placebo not comprising the GLP-1 receptor agonist.
75. The method according to any one of the preceding embodiments wherein said reduction is at least 5%, such as at least 10% or at least 15%.
76. The method according to any one of the preceding embodiments wherein the GLP-1 receptor agonist is a GLP-1 fragment, derivative or analogue.
77. The method according to any one of the preceding embodiments wherein the GLP-1 receptor agonist comprises no more than 5, such as no more than 4 or no more than 3, amino acid residues which have been substituted, inserted or deleted as compared to GLP-1(7-37).
78. The method according to any one of the preceding embodiments wherein the GLP-1 receptor agonist is selected from the group consisting of: GLP-1(7-37); GLP-1(7-36) amide; Exenatide; Exenatide LAR; Liraglutide; Semaglutide; Taspoglutide; Albiglutide; Lixisenatide; and Dulaglutide.
79. The method according to any one of the preceding embodiments wherein the GLP-1 receptor agonist is Liraglutide.
80. The method according to any one of the preceding embodiments for use in treating hypoglycaemia or hypoglycaemic episodes associated with insulin therapy of type 1 diabetes.
81. The method according to any one of the preceding embodiments wherein the GLP-1 receptor agonist is for use in the treatment or prevention of one or more additional symptoms associated with insulin therapy of type 1 diabetes, wherein said one or more additional symptoms associated with insulin therapy of type 1 diabetes may be selected from the group consisting of: reduced glycaemic control; insulin deficiency; and obesity.
82. The method according to any one of the preceding embodiments wherein the treatment or prevention of hypoglycaemia or hypoglycaemic episodes in patients suffering from type 1 diabetes comprises or consists of treating or preventing one or more symptom selected from the group consisting of: hypoglycaemia; overall hypoglycaemic episodes; nocturnal hypoglycaemic episodes; and daytime hypoglycaemic episodes.
83. The method according to any one of the preceding embodiments wherein the type 1 diabetes exhibits complete or partial insulin deficiency; and/or absolute, or substantially absolute, insulin deficiency.
84. The method according to any one of the preceding embodiments wherein the GLP-1 receptor agonist is formulated together with a pharmaceutically acceptable carrier, vehicle, diluent and/or excipient.
85. The method according to any one of the preceding embodiments wherein the GLP-1 receptor agonist is for use in combination with one or more additional anti-diabetic drugs, such as insulin.
86. The method according to any one of the preceding embodiments wherein the method and/or the GLP-1 receptor agonist is as defined herein.

### Additional Non-Limiting List of Embodiments

1. A GLP-1 receptor agonist for use in treating or preventing hypoglycaemia or hypoglycaemic episodes in patients suffering from type 1 diabetes.
2. The GLP-1 receptor agonist for use according to Embodiment 1 wherein the GLP-1 receptor agonist is suitable for once daily administration.
3. The GLP-1 receptor agonist for use according to Embodiment 1 wherein the GLP-1 receptor agonist is suitable for once weekly administration.
4. The GLP-1 receptor agonist for use according to Embodiment 1 wherein the GLP-1 receptor agonist is a GLP-1 fragment, derivative or analogue.
5. The GLP-1 receptor agonist for use according to Embodiment 1 wherein the GLP-1 derivative or analogue is selected from the group consisting of: GLP-1(7-37); GLP-1(7-36) amide; Exenatide (Byetta®); Exenatide LAR (BYDUREON®); Liraglutide (Victoza®); Semaglutide; Taspoglutide; Albiglutide; Lixisenatide (Sanofi®); and Dulaglutide.
6. The GLP-1 receptor agonist for use according to Embodiment 1 wherein the GLP-1 derivative or analogue is selected from the group consisting of: Liraglutide (Victoza®) and Semaglutide.
7. The GLP-1 receptor agonist for use according to Embodiment 1 wherein GLP-1 derivative or analogue is a long-acting GLP-1 derivative or analogue.
8. The GLP-1 receptor agonist for use according to Embodiment 7 wherein the long-acting GLP-1 derivative or analogue is selected from the group consisting of: Liraglutide (Victoza®); Semaglutide; Taspoglutide; Albiglutide; Taspoglutide; Dulaglutide; and Exenatide LAR.
9. The GLP-1 receptor agonist for use according to Embodiment 7 wherein the long-acting GLP-1 derivative or analogue is Liraglutide (Victoza™)
10. The GLP-1 receptor agonist for use according to Embodiment 7 wherein the long-acting GLP-1 derivative or analogue is Semaglutide.
11. The GLP-1 receptor agonist according to any one of the preceding embodiments for use in treating hypoglycaemia or hypoglycaemic episodes associated with insulin therapy of type 1 diabetes.
12. The GLP-1 receptor agonist for use according to any one of the preceding embodiments wherein the GLP-1 receptor agonist is for use in the treatment or prevention of one or more additional symptoms associated with insulin therapy of type 1 diabetes.
13. The GLP-1 receptor agonist for use according to Embodiment 12, wherein the one or more additional symptoms associated with insulin therapy of type 1 diabetes are selected from the group consisting of: reduced glycaemic control; insulin deficiency; and obesity.
14. The GLP-1 receptor agonist for use according to any one of the preceding embodiments wherein the treatment or prevention of hypoglycaemia or hypoglycaemic episodes in patients suffering from type 1 diabetes comprises or consists of treating or preventing one or more symptom selected from the group consisting of: hypoglycaemia; overall hypoglycaemic episodes; nocturnal hypoglycaemic episodes; and daytime hypoglycaemic episodes.
15. The GLP-1 receptor agonist for use according to Embodiment 14 wherein the GLP-1 receptor agonist is capable of reducing overall nocturnal hypoglycaemic episodes.
16. The GLP-1 receptor agonist for use according to Embodiment 14 wherein the GLP-1 receptor agonist is capable of reducing the duration of nocturnal hypoglycaemic episodes.
17. The GLP-1 receptor agonist for use according to Embodiment 14 wherein the GLP-1 receptor agonist is capable of reducing overall nocturnal hypoglycaemic episodes by at least 25%, for example, 30%, 35%, or 40%.
18. The GLP-1 receptor agonist for use according to Embodiment 14 wherein the GLP-1 receptor agonist is capable of reducing overall nocturnal hypoglycaemic episodes by at least 31%.
19. The GLP-1 receptor agonist for use according to Embodiment 14 wherein the GLP-1 receptor agonist is capable of reducing overall nocturnal hypoglycaemic episodes by at least 35%.
20. The GLP-1 receptor agonist for use according to Embodiment 14 wherein the GLP-1 receptor agonist is capable of reducing overall nocturnal hypoglycaemic episodes by at least 39%.
21. The GLP-1 receptor agonist for use according to Embodiment 18 wherein the dose of GLP-1 receptor agonist is 0.6 mg/day.
22. The GLP-1 receptor agonist for use Embodiment 19 wherein the dose of GLP-1 receptor agonist is 1.2 mg/day.
23. The GLP-1 receptor agonist for use according to Embodiment 20 wherein the dose of GLP-1 receptor agonist is 1.2 mg/day.
24. The GLP-1 receptor agonist for use according to any of the preceding embodiments wherein the GLP-1 receptor agonist is capable of reducing the amount of exogenous glucose needed for recovering from a hypoglycaemic episode and/or reducing the severity of blood glucose excursions.
25. The GLP-1 receptor agonist for use according to any of the preceding embodiments wherein the GLP-1 receptor agonist, when administered at a dose 1.2 mg/mL once daily, is capable of reducing the amount of exogenous glucose needed for recovering after a hypoglycaemic episode by at least 19%.
26. The GLP-1 receptor agonist for use according to any of Embodiments 1 to 24 wherein the GLP-1 receptor agonist, when administered in a dose 0.6 mg/mL once daily, is capable of reducing the amount of exogenous glucose needed for recovering after a hypoglycaemic episode by at least 23%.
27. The GLP-1 receptor agonist for use according to any of Embodiments 1 to 24 wherein the GLP-1 receptor agonist, when administered in a dose 1.8 mg/mL once daily, is capable of reducing the amount of exogenous glucose needed for recovering after a hypoglycaemic episode by at least about 37% relative to placebo.
28. The GLP-1 receptor agonist for use according to any one of the preceding embodiments wherein the GLP-1 receptor agonist is administered at a dose sufficient to reduce the exogenous glucose requirement of the patient to recover from a hypoglycaemic episode.
29. The GLP-1 receptor agonist for use according to any one of the preceding embodiments wherein the GLP-1 receptor agonist is administered at a dose sufficient to maximally reduce the exogenous glucose requirement in the patient to recover from a potential hypoglycaemic episode.
30. The GLP-1 receptor agonist for use according to any one of Embodiments 1 to 4 and 11 to 29 wherein the GLP-1 receptor agonist is semaglutide.
31. The GLP-1 receptor agonist for use according to any one of Embodiments 1 to 4 and 11 to 29 wherein the GLP-1 receptor agonist administered is liraglutide and the dose is sufficient to optimally reduce the exogenous glucose requirement in the patient recovering from potential hypoglycaemic episodes is between 0.6 mg per day and 1.8 mg per day (for example, between 0.6 mg and 1.2 mg per day).
32. The GLP-1 receptor agonist for use according to any one of Embodiments 1 to 4 and 11 to 29 wherein the GLP-1 receptor agonist is liraglutide and the dose sufficient to optimally reduce the exogenous glucose requirement in the patient recovering from potential hypoglycaemic episodes is selected from the group consisting of 0.6 mg per day; 1.2 mg per day and 1.8 mg per day.
33. The GLP-1 receptor agonist for use according to any one of Embodiments 1 to 4 and 11 to 29 wherein the GLP-1 receptor agonist is administered at a dose of 0.6 mg/mL once daily to reduce the exogenous glucose requirement of the patient to recover from a hypoglycaemic episode.
34. The GLP-1 receptor agonist for use according to any one of Embodiments 1 to 4 and 11 to 29 wherein the GLP-1 receptor agonist is administered at a dose of 1.2 mg/mL once daily to reduce the exogenous glucose requirement of the patient to recover from a hypoglycaemic episode.
35. The GLP-1 receptor agonist for use according to any one of Embodiments 1 to 4 and 11 to 29 wherein the GLP-1 receptor agonist is administered at a dose of 1.8 mg/mL once daily to reduce the exogenous glucose requirement of the patient to recover from a hypoglycaemic episode.
36. The GLP-1 receptor agonist for use according to any one of the preceding embodiments wherein the type 1 diabetes exhibits complete or partial insulin deficiency.
37. The GLP-1 receptor agonist for use according to any one Embodiments 1 to 36 wherein the type 1 diabetes exhibits absolute, or substantially absolute, insulin deficiency.
38. The GLP-1 receptor agonist for use according to any one of the preceding embodiments wherein the GLP-1 receptor agonist is formulated together with a pharmaceutically acceptable carrier, vehicle, diluent and/or excipient.
39. The GLP-1 receptor agonist for use according to any one of the preceding embodiments wherein the GLP-1 receptor agonist is for use in treatment period of greater than one day (for example, a period of, or at least of, 2 days, a period of, or at least of, 3 days, a period of, or at least of, 4 days, a period of, or at least of, 5 days, a period of, or at least of, 6 days, a period of, or at least of, 1 week, a period of, or at least of, 2 weeks, a period of, or at least of, 3 weeks, a period of, or at least of, 4 weeks, a period of, or at least of, 1 month, a period of, or at least of, 2 months, a period of, or at least of, 3 months, a period of, or at least of, 4 months, a period of, or at least of, 5 months, a period of, or at least of, 6 months, and a continuous or indefinite period).
40. The GLP-1 receptor agonist for use according to any one of the preceding embodiments wherein the GLP-1 receptor agonist is for use in a daily treatment.
41. The GLP-1 receptor agonist for use according to any one of the preceding embodiments wherein the GLP-1 receptor agonist is for use in a weekly treatment.
42. The GLP-1 receptor agonist for use according to any one of the preceding embodiments wherein the GLP-1 receptor agonist is for use in combination with one or more additional anti-diabetic drug.
43. The GLP-1 receptor agonist for use according to Embodiment 42 wherein the one or more further or additional drug is administered, concurrently or consecutively, with the GLP-1 receptor agonist for use.
44. The GLP-1 receptor agonist for use according to Embodiment 43 wherein the one or more additional anti-diabetic drug is administered, concurrently or consecutively, with the GLP-1 receptor agonist for use.
45. The GLP-1 receptor agonist for use according to Embodiment 44 wherein the one or more additional anti-diabetic drug is administered, concurrently with the GLP-1 receptor agonist for use in the same formulation.
46. The GLP-1 receptor agonist for use according to any one of Embodiments 42 to 45 wherein the one or more additional anti-diabetic drug is selected from the group consisting of: Insulins; and Sensitizers (such as biguanides and thiazolidinediones).
47. The GLP-1 receptor agonist for use according to Embodiment 46 wherein the one or more anti-diabetic drug is, or includes, an insulin.
48. The use of a GLP-1 agonist in the preparation of a medicament for treating or preventing hypoglycaemia or hypoglycaemic episodes in patients suffering from type 1 diabetes.
49. A method of treating or preventing hypoglycaemia or hypoglycaemic episodes in patients suffering from type 1 diabetes comprising administering an effective amount of a GLP-1 agonist to an individual in need of treatment.

### EXAMPLES

The invention will now be described in more detail with the aid of the following Examples and Figures.

### Abbreviations

CGM=continuous glucose monitoring

IG=interstitial glucose

PDE=person days of exposure

### Example A

### Liraglutide as Adjunct to Insulin in Type 1 Diabetes: Effects on Glycaemic Control and Safety in a Randomized, Double-Blind Crossover Trial

### Introduction

Small-scale studies and single case reports in subjects with type 1 diabetes indicate that treatment with a GLP-1 agonist¹, such as liraglutide^{2,3}, may result in:
Improvement in glycaemic control
   Reduction of glucose excursions
Reduction in frequency of hypoglycaemia
Reduction in insulin requirements
Weight reduction

In a trial investigating the effects of liraglutide treatment adjunct to insulin on counterregulatory responses during a hypoglycaemic clamp, we also assessed glycaemic control and safety during the 4-week treatment period.

### Methods

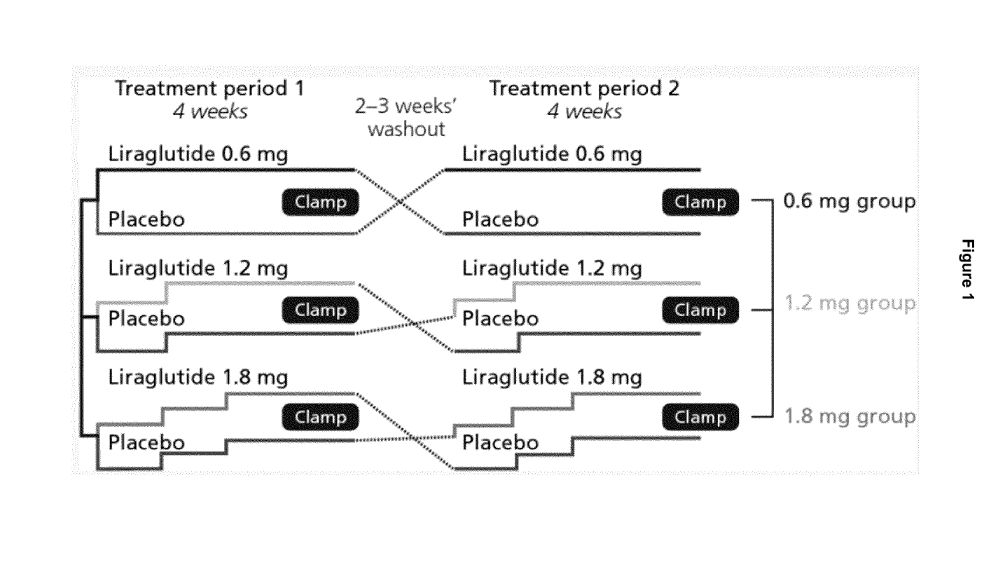

45 adults with type 1 diabetes were randomised to 1 of 3 dose groups of liraglutide adjunct to insulin or placebo adjunct to insulin and 1 of 2 sequences (liraglutide/placebo or placebo/liraglutide) in a crossover design with a wash-out period between treatments (FIG. 1).

Liraglutide starting dose was 0.6 mg/day with weekly increases of 0.6 mg until target dose was reached. Subjects received target dose for a minimum of 2 weeks.

After 4 weeks of treatment, estimated treatment differences in insulin dose, HbA1c, pulse and body weight were assessed. Hypoglycaemia and hypoglycaemic episodes and overall safety parameters were assessed throughout the treatment period.

### Results

All results are for liraglutide adjunct to insulin compared to placebo adjunct to insulin. Subject characteristics at baseline were comparable between groups (Table 1).

### Insulin Dose

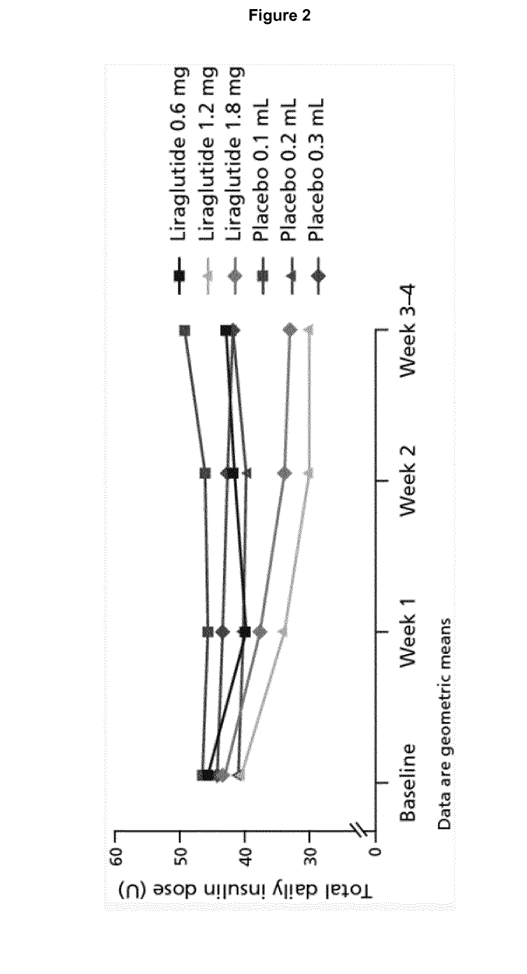

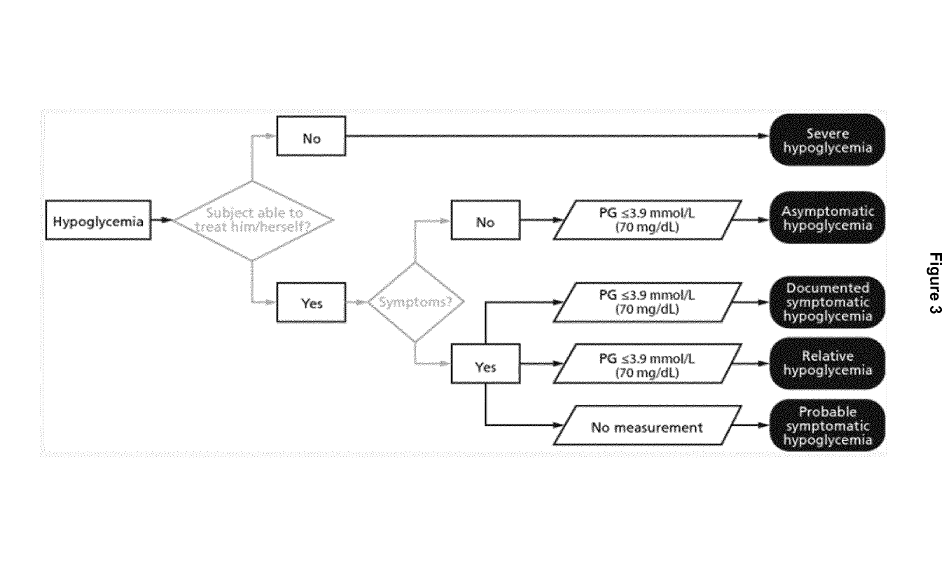

Daily insulin dose was reduced by 27% with 1.2 mg liraglutide (p<0.001) and by 24% with 1.8 mg liraglutide (p<0.001). No difference was seen between liraglutide 0.6 mg and placebo (FIG. 2).

### HbA1c

No changes and no difference in changes in HbA1c from baseline and after 4 weeks of treatment were seen for liraglutide compared to placebo.

Hypoglycaemia and/or Hypoglycaemic Episodes

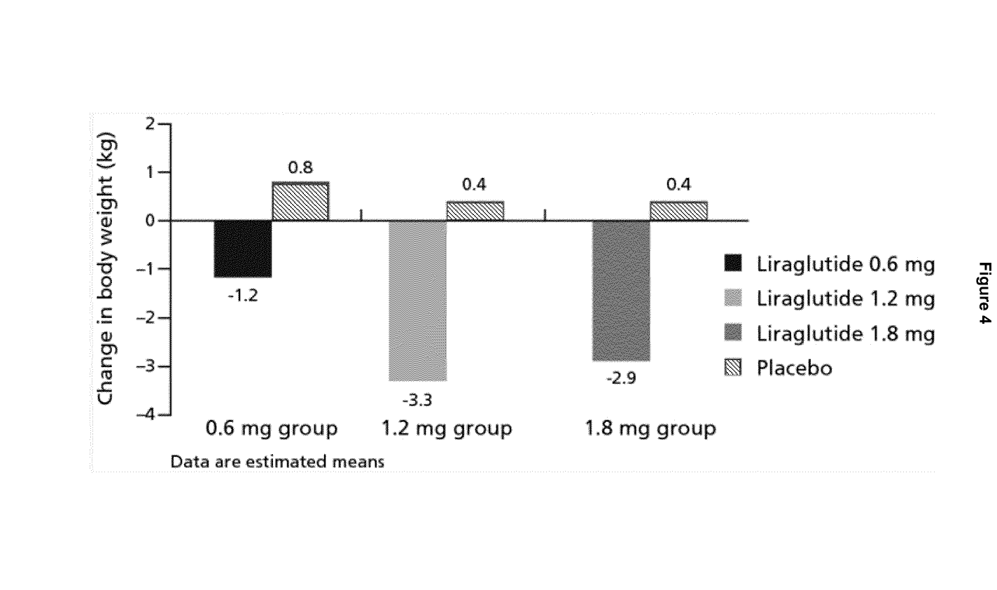

Hypoglycaemia and/or hypoglycaemic episodes were defined and assessed according to the ADA Hypoglycaemic Classification (FIG. 3).

No systematic differences in number of subjects with hypoglycaemias or number of hypoglycaemias were seen across treatments. No systematic differences in number of subjects with hypoglycaemic episodes or number of hypoglycaemic episodes were seen across treatments. None of the hypoglycaemias or hypoglycaemic episodes were severe (Table 2).

### Adverse Events

The number of adverse events (AEs) was higher during liraglutide treatment compared to placebo (Table 3).

The difference was due to more gastrointestinal (GI) AEs with liraglutide. An increasing number of GI AEs was seen with increasing dose. Nausea was most frequently reported. All GI AEs were mild or moderate in severity.

### Vital Signs

No clinically significant findings in vital signs (including blood pressure, pulse and body temperature) were reported during this trial.

### Body Weight

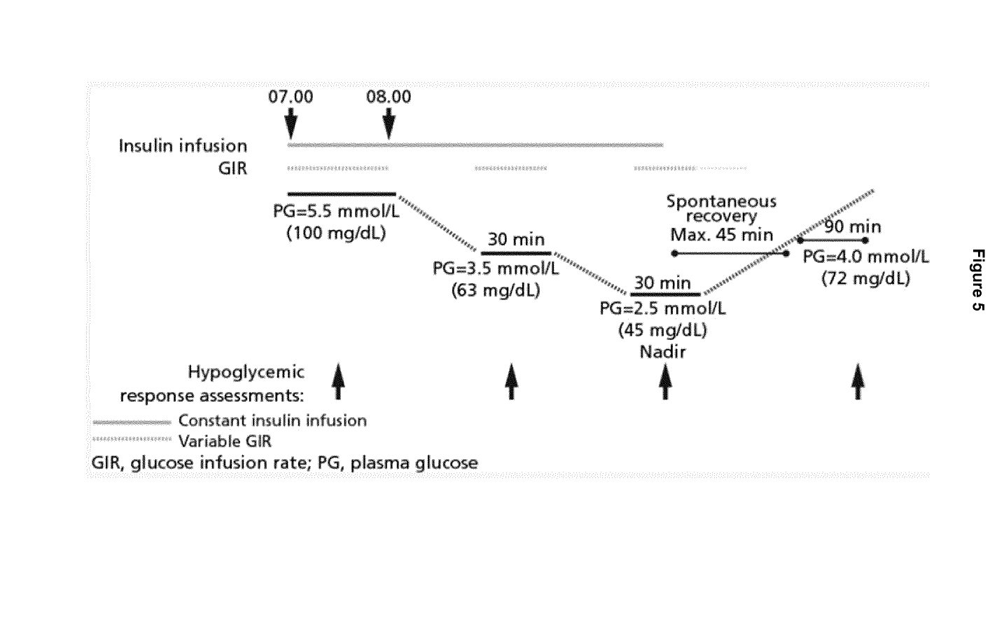

A statistically significant reduction in body weight was seen with all liraglutide groups compared to placebo. Estimated treatment differences: 0.6 mg group=−2.0 kg, p<0.001; 1.2 mg group=−3.7 kg, p<0.001; 1.8 mg group=−3.3 kg, p<0.001 (FIG. 4).

### Conclusions

A significant reduction in insulin dose after liraglutide treatment with 1.2 mg and 1.8 mg indicates that liraglutide adjunct to insulin has a pharmacologic effect in type 1 diabetes that may contribute to glycaemic control.

Liraglutide adjunct to insulin did not affect the number of hypoglycaemias or overall number of hypoglycaemic episodes.

No unexpected liraglutide-related safety or tolerability issues were identified in this short-term trial in subjects with type 1 diabetes.

The observed weight loss with liraglutide is of potential clinical benefit to subjects with type 1 diabetes.

### REFERENCES

1) Kielgast et al. Diabetes 2011; 60(5): 1599-1607;
2) Kielgast et al. Diabetes Care 2011; 34(7): 1463-1468
3) Varanasi et al. Eur J Endocrinol 2011; 165(1): 77-84.

### Example B

### Effects of Liraglutide as Adjunct to Insulin on Counterregulatory Hormone Responses to Hypoglycaemia in Type 1 Diabetes: A Randomized, Double-Blind Crossover Trial

### Introduction

Incretin-based therapy may reduce glucagon levels in subjects with type 1 diabetes¹⁻³.

Glucagon is an important counterregulatory hormone to hypoglycaemia, but glucagon response to hypoglycaemia may be deficient in subjects with type 1 diabetes⁴. If the glucagon response is further compromised by treatment with a GLP-1 receptor agonist, such as a GLP-1 analogue, it could be a limiting factor for the safe use of liraglutide as an adjunct to insulin treatment.

This trial served to investigate the counterregulatory hormone response to hypoglycaemia in subjects with type 1 diabetes following 4 weeks of treatment with liraglutide as adjunct to insulin treatment.

### Methods

45 adults with type 1 diabetes were randomised to 1 of 3 dose groups of liraglutide adjunct to insulin or placebo adjunct to insulin, and 1 of 2 sequences (liraglutide/placebo or placebo/liraglutide) in a crossover design with a wash-out period between treatments (FIG. 1).

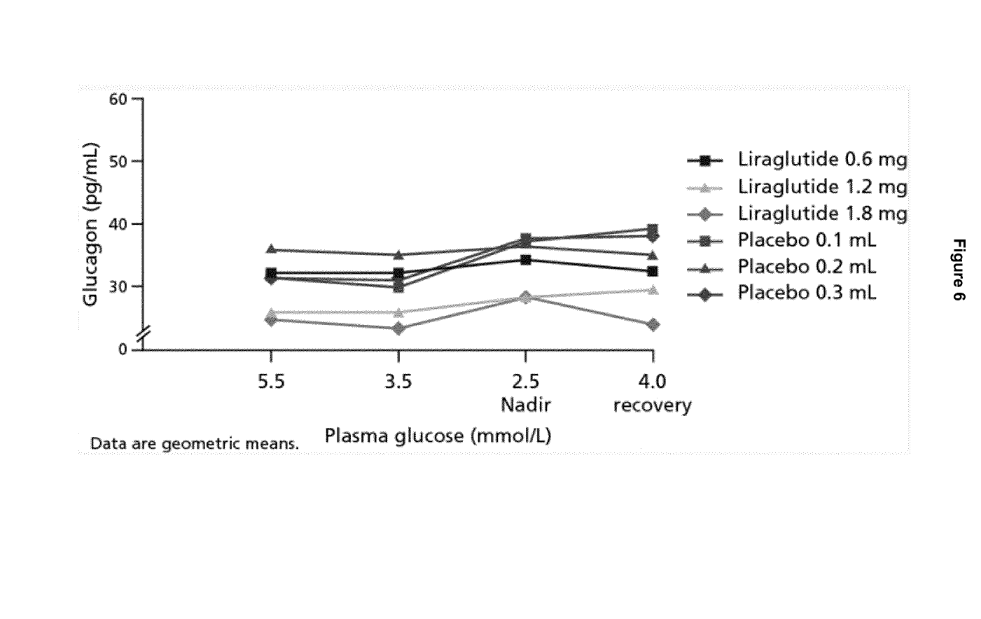

A stepwise hypoglycaemic clamp was performed at the end of each treatment period via controlled i.v. insulin/glucose infusion (FIG. 5). The successive plasma glucose (PG) levels were 5.5, 3.5, 2.5 mmol/L (nadir), and 4.0 mmol/L (recovery). Counterregulatory hormones (glucagon, adrenalin, noradrenalin, cortisol, and growth hormone) and vital signs (pulse, blood pressure) were measured at each successive PG level during the clamp. Glucose infusion rate was also assessed.

### Results

All results are for liraglutide adjunct to insulin compared to placebo adjunct to insulin.

Subject characteristics at baseline were comparable between groups: Age: 34.5±11.2 years, BMI: 23.9±2.4 kg/m2, HbA1c: 7.6±0.8%, duration of diabetes: 16.6±9.4 years [mean±SD] (see Table 1).

### Counterregulatory Response

Little glucagon response to hypoglycaemia was seen in all groups with no systematic differences between treatments (FIG. 6).

No significant differences in glucagon were seen with liraglutide vs. placebo at nadir PG: 0.6 mg group=0.91 [0.66;1.25], 1.2 mg group=0.77 [0.56;1.08], 1.8 mg group=0.76 [0.55;1.05] (ratio [95% CI]). However, there was a trend toward lower glucagon levels at nadir and other PG levels in the 1.2 mg and 1.8 mg groups (FIG. 6).

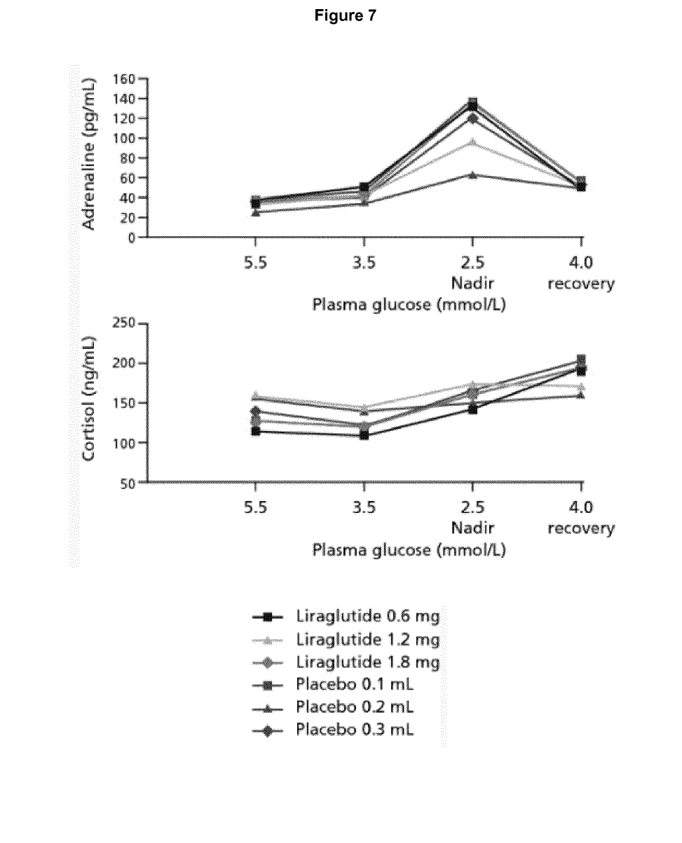

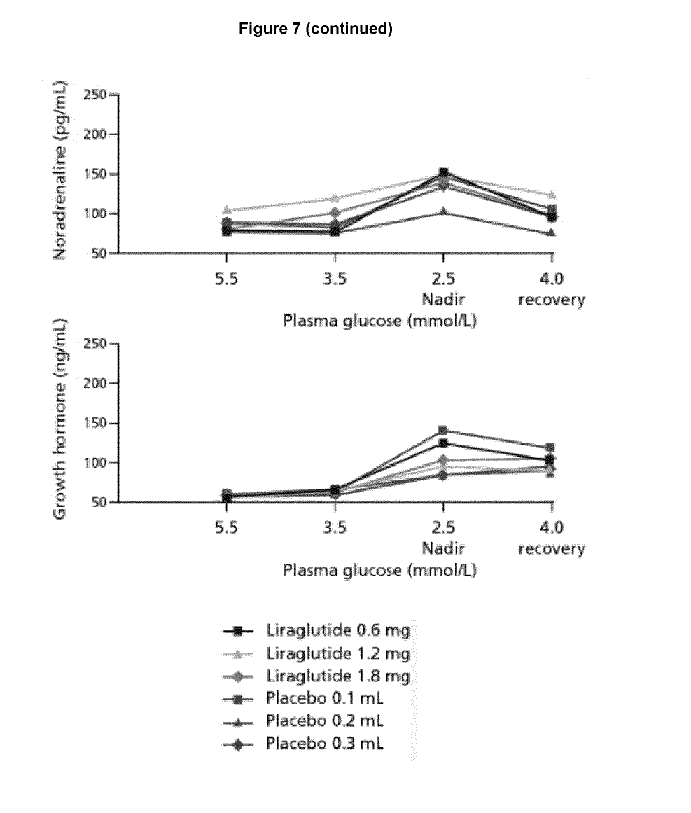

Increased concentration of other counterregulatory hormones to hypoglycaemia was seen, but no systematic differences were observed between liraglutide and placebo (FIG. 7).

### Vital Signs

Pulse was significantly higher at all plasma glucose levels for all liraglutide doses compared to placebo. However, when looking at the change in pulse (beats per minute [BPM]), there was no significant difference from plasma glucose level 5.5 mmol/L to nadir level between liraglutide and placebo: 0.6 mg group=0.6 BPM [−5.5;6.7], 1.2 mg group=2.3 BPM [−4.0;8.7], 1.8 mg group=−3.4 BPM [−9.6;2.9] (estimated treatment difference [95% CI]).

There were no systematic differences in systolic and diastolic blood pressure between liraglutide and placebo during the hypoglycaemic clamp.

### Glucose Infusion Rate

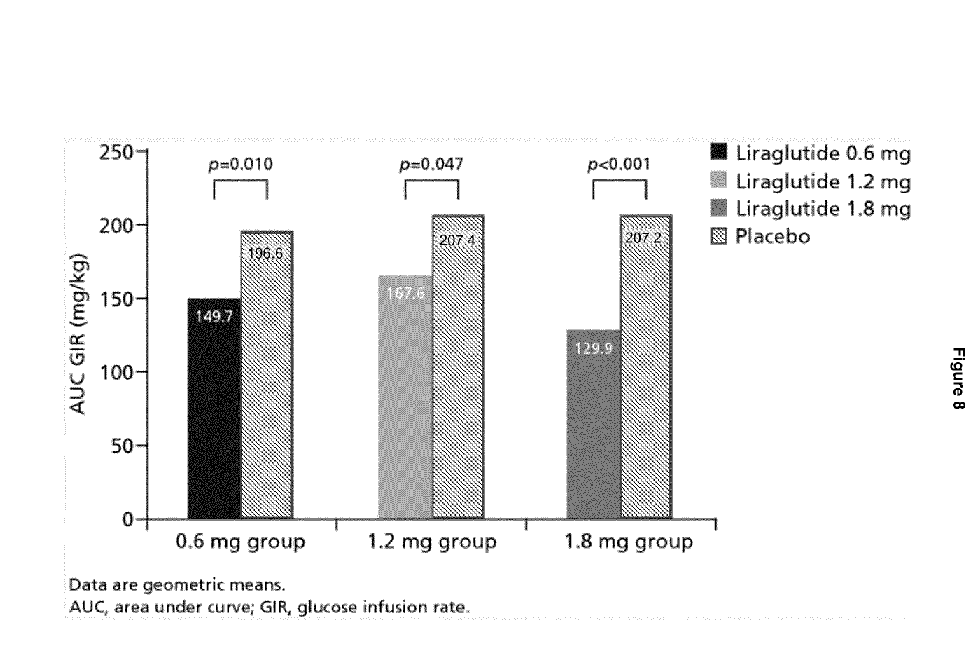

Area under the curve for glucose infusion rate during the hypoglycaemic clamp was significantly lower for all liraglutide dose groups compared to placebo: 0.6 mg group=0.76 [0.62;0.93] p=0.010, 1.2 mg group=0.81 [0.66;1.00] p=0.047, 1.8 mg group=0.63 [0.51;0.77] p<0.001 (ratio [95% CI] p-value) (FIG. 8).

Number of Hypoglycaemia and/or Hypoglycaemic Episodes

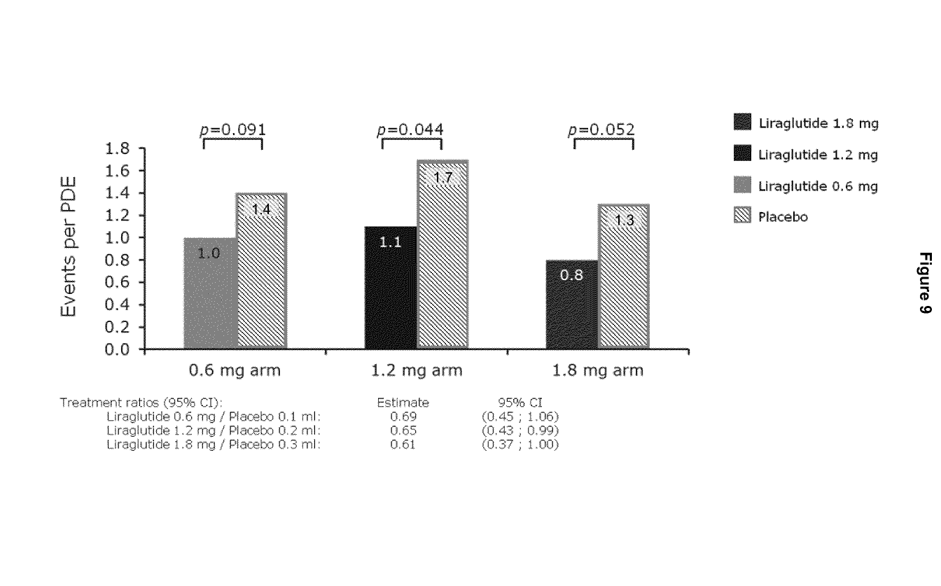

The number of hypoglycaemic events (defined as IG mmol/L) was analysed by a negative binomial regression model with a log link and log (duration of CGM [day]) as offset. The model included treatment and period as fixed factors and subject as a random effect. CGM=continuous glucose monitoring; IG=interstitial glucose; PDE=person days of exposure. The results are shown in FIG. 9.

Lower amount of hypos less than 3.9 mmol/L during continuous glucose monitoring for liraglutide vs placebo in all doses were statistically significant for 1.2 mg.

The estimate was 0.65 as seen in FIG. 9 which corresponds to a 35% lower number of hypos with liraglutide 1.2 mg vs placebo. However 0.6 (0.69=31% lower) is very close to statistical significance. 1.8 mg lira (0.61=39% lower) significance level was set to p=0.050.

A lower number of hypoglycaemic values≦3.9 mmol/L was seen for all liraglutide groups; for liraglutide 1.2 mg, the number was statistically significantly lower compared to placebo (1.1 versus 1.7 event per 1 patient day exposure, p=0.044). Similar results were seen with values<3.1 mmol/L.

Duration of Hypoglycaemia and/or Hypoglycaemic Episodes

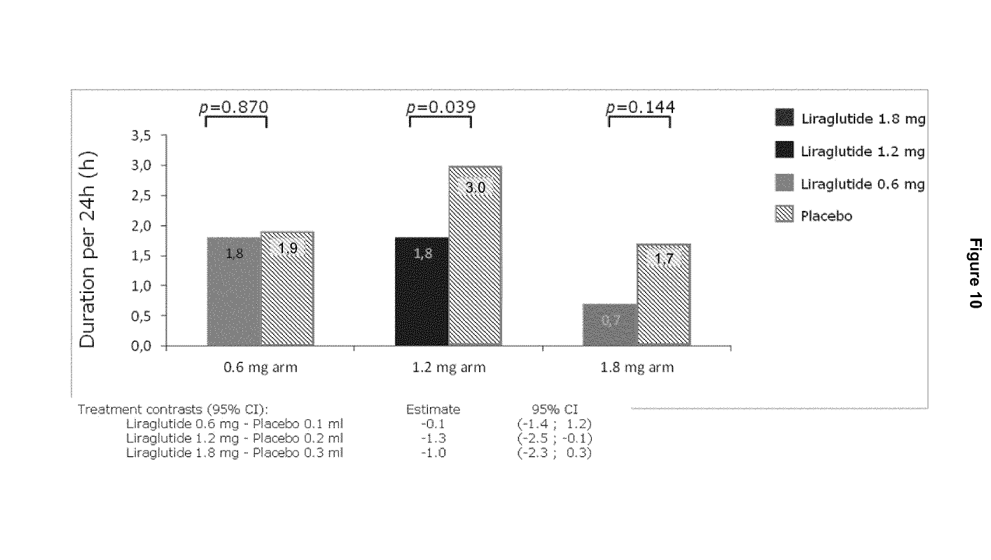

The duration of hypoglycaemic episodes (IG≦3.9 mmol/L) during CGM was analysed and the results are shown in FIG. 10. The duration of hypoglycaemic episodes was reduced for liraglutide vs. placebo. This trend was seen for the doses 0.6 mg and 1.8 mg and in 1.2 mg the results were statistically significant, significance level was set to p=0.050.

### Conclusions

No safety concerns related to counterregulatory response to hypoglycaemia were raised with liraglutide as adjunct to insulin treatment in subjects with type 1 diabetes.

Subjects with type 1 diabetes treated with liraglutide adjunct to insulin needed less glucose to obtain the selected plasma glucose levels throughout the hypoglycaemic clamp.

Subjects with type 1 diabetes treated with liraglutide adjunct to insulin had fewer hypoglycaemic episodes.

Subjects with type 1 diabetes treated with liraglutide adjunct to insulin had shorter duration of hypoglycaemic episodes.

### REFERENCES

1) Kielgast et al. Diabetes 2011; 60(5): 1599-1607
2) Kielgast et al. Diabetes Care 2011; 34(7): 1463-1468
3) Varanasi et al. Eur J Endocrinol 2011; 165(1): 77-84
4) Cryer P E. Endocrinol Metab Clin North Am 2010; 39(3): 641-654.

### Example C

### General Methods

The following methods may be used in determining one or more factors described in respect of the present invention.

### In Vitro Potency (AlphaScreen, Membranes)

The purpose of this assay is to test the activity, or potency, of the GLP-1 receptor agonist, such as GLP-1 derivatives, in vitro. The potencies of GLP-1 receptor agonists, such as GLP-1 derivatives, may be determined as described below, i.e. as the stimulation of the formation of cyclic AMP (cAMP) in a medium containing membranes expressing the human GLP-1 receptor.

### Principle

Purified plasma membranes from a stable transfected cell line, BHK467-12A (tk-ts13), expressing the human GLP-1 receptor is stimulated with the GLP-1 receptor agonists, such as a GLP-1 analogue or derivative, in question, and the potency of cAMP production is measured using the AlphaScreen™ cAMP assay kit from Perkin Elmer Life Sciences. The basic principle of The AlphaScreen assay is a competition between endogenous cAMP and exogenously added biotin-cAMP. The capture of cAMP is achieved by using a specific antibody conjugated to acceptor beads.

### Cell Culture and Preparation of Membranes

A stably transfected cell line and a high expressing clone is selected for screening. The cells are grown at 5% CO₂ in DMEM, 5% FCS, 1% Pen/Strep (Penicillin/Streptomycin) and 0.5 mg/ml of the selection marker G418.

Cells at approximate 80% confluence are washed twice with PBS and harvested with Versene (aqueous solution of the tetrasodium salt of ethylenediaminetetraacetic acid), centrifuged 5 min at 1000 rpm and the supernatant is removed. The additional steps are all made on ice. The cell pellet is homogenised by the Ultrathurax for 20-30 s in 10 ml of Buffer 1 (20 mM Na-HEPES, 10 mM EDTA, pH=7.4), centrifuged 15 min at 20,000 rpm and the pellet resuspended in 10 ml of Buffer 2 (20 mM Na-HEPES, 0.1 mM EDTA, pH=7.4). The suspension is homogenised for 20-30 s and centrifuged 15 min at 20,000 rpm. Suspension in Buffer 2, homogenisation and centrifugation is repeated once and the membranes resuspended in Buffer 2. The protein concentration is determined and the membranes stored at −80° C. until use.

The assay is performed in flat-bottom 96-well plates (Costar cat. no: 3693). The final volume per well is 50 μl.

### Solutions and Reagents

AlphaScreen cAMP assay kit from Perkin Elmer Life Sciences (cat. No: 6760625M); containing Anti-cAMP Acceptor beads (10 U/μl), Streptavidin Donor beads (10 U/μl) and Biotinylated-cAMP (133 U/μl).

AlphaScreen Buffer, pH=7.4: 50 mM Tris-HCl (Sigma, cat. no: T3253); 5 mM HEPES (Sigma, cat. no: H3375); 10 mM MgCl₂, 6H₂O (Merck, cat. no: 5833); 150 mM NaCl (Sigma, cat. no: S9625); 0.01% Tween (Merck, cat. no: 822184). The following was added to the AlphaScreen Buffer prior to use (final concentrations indicated): BSA (Sigma, cat. no. A7906): 0.1%; IBMX (Sigma, cat. no. 15879): 0.5 mM; ATP (Sigma, cat. no. A7699): 1 mM; GTP (Sigma, cat. no. G8877): 1 μM.

cAMP standard (dilution factor in assay=5): cAMP Solution: 5 μL of a 5 mM cAMP-stock+495 μL AlphaScreen Buffer.

Suitable dilution series in AlphaScreen Buffer is prepared of the cAMP standard as well as the GLP-1 receptor agonist, such as the GLP-1 analogue or derivative, to be tested, e.g. the following eight concentrations of the GLP-1 compound: 10⁻⁷, 10⁻⁸, 10⁻⁹, 10⁻¹⁰, 10⁻¹¹, 10⁻¹², 10⁻¹³ and 10⁻¹⁴M, and a series from, e.g., 10⁻⁶ to 3×10⁻¹¹ of cAMP.

### Membrane/Acceptor Beads

Membranes are prepared from hGLP-1/BHK 467-12A cells with a concentration of 6 μg/well corresponding to 0.6 mg/ml (the amount of membranes used pr. well may vary)

“No membranes”: Acceptor Beads (15 μg/ml final) in AlphaScreen buffer

“6 μg/well membranes”: membranes+Acceptor Beads (15 μg/ml final) in AlphaScreen buffer

An aliquot (10 μl) of “No membranes” added to the cAMP standard (per well in duplicates) and the positive and negative controls

An aliquot 10 μl of “6 μg/well membranes” was added to GLP-1 and analogues (per well in duplicate or triplicate wells)

Pos. Control: 10 μl “no membranes”+10 μl AlphaScreen Buffer

Neg. Control: 10 μl “no membranes”+10 μl cAMP Stock Solution (50 μM)

As the beads are sensitive to direct light, any handling is in the dark (as dark as possible), or in green light. All dilutions made on ice.

### Procedure

1. Make the AlphaScreen Buffer.

2. Dissolve and dilute the GLP-1/Analogues/cAMP standard in AlphaScreen Buffer.

3. Make the Donor Beads Solution by mixing streptavidin donor beads (2 units/well) and biotinylated cAMP (1.2 units/well) and incubate 20-30 min in the dark at room temperature.

4. Add the cAMP/GLP-1/Analogues to the plate: 10 μl per well.

5. Prepare membrane/Acceptor Beads solution and add this to the plates: 10 μl per well.

6. Add the Donor Beads: 30 μl per well.

7. Wrap the plate in aluminium foil and incubate on the shaker for 3 hours (very slowly) at RT.

8. Count on AlphaScreen—each plate pre incubates in the AlphaScreen for 3 minutes before counting.

### In Vitro Potency (CRE Luciferase; Whole Cells)

The purpose of this example is to test the activity, or potency, of the GLP-1 receptor agonist, such as GLP-1 derivatives, in vitro. The in vitro potency is the measure of human GLP-1 receptor activation in a whole cell assay. This method is preferred to that described above.

The potencies of GLP-1 receptor agonist, such as GLP-1 derivatives, may be determined as described below.

### Principle

In vitro potency is determined by measuring the response of the human GLP-1 receptor in a reporter gene assay. The assay is performed in a stably transfected BHK cell line that expresses the human GLP-1 receptor and contains the DNA for the cAMP response element (CRE) coupled to a promoter and the gene for firefly luciferase (CRE luciferase). When the human GLP-1 receptor is activated it results in the production of cAMP, which in turn results in the luciferase protein being expressed. When assay incubation is completed the luciferase substrate (luciferin) is added and the enzyme converts luciferin to oxyluciferin to produce bioluminescence. The luminescence is measured as the readout for the assay.

In order to test the binding of the GLP-1 receptor agonists, such as the derivatives, to albumin, the assay is performed in the absence of serum albumin as well as in the presence of a considerably higher concentration of serum albumin (1.0% final assay concentration). An increase of the in vitro potency, EC₅₀ value, in the presence of serum albumin would indicate an affinity to serum albumin and represents a method to predict a protracted pharmacokinetic profile of the test substance in animal models.

### Cell Culture and Preparation

The cells used in this assay (clone FCW467-12A/KZ10-1) are BHK cells with BHKTS13 as a parent cell line. The cells were derived from a clone (FCW467-12A) that expresses the human GLP-1 receptor and are established by further transfection with CRE luciferase to obtain the current clone.

The cells are cultured at 5% CO₂ in Cell Culture Medium. They are aliquoted and stored in liquid nitrogen. Before each assay an aliquot is taken up and washed twice in PBS before being suspended at the desired concentration in the assay specific buffer. For 96-well plates the suspension is made to give a final concentration of 5×10³ cells/well.

### Materials

The following chemicals are used in the assay: Pluronic F-68 (10%) (Gibco 2404), human serum albumin (HSA) (Sigma A9511), ovalbumin (Sigma A5503), DMEM w/o phenol red (Gibco 11880-028), 1 M Hepes (Gibco 15630), Glutamax 100x (Gibco 35050) and steadylite plus (PerkinElmer 6016757).

### Buffers

Cell Culture Medium consisted of DMEM medium with 10% FBS (Fetal Bovine Serum; Invitrogen 16140-071), 1 mg/ml G418 (Invitrogen 15140-122), 240 nM MTX (methotrexate; Sigma M9929) and 1% pen/strep (penicillin/streptomycin; Invitrogen 15140-122).

Assay Medium consisted of DMEM w/o phenol red, 10 mM Hepes and 1× Glutamax. The 1% Assay Buffer consisted of 2% ovalbumin, 0.2% Pluronic F-68 and 2% HSA in assay medium. The 0% Assay Buffer consisted of 2% ovalbumin and 0.2% Pluronic F-68 in Assay Medium.

### Procedure

1) Cell stocks thawed in a 37° C. water bath.

2) Cells washed three times in PBS.

3) The cells were counted and adjusted to 5×10³ cells/50 μl (1×10⁵ cells/ml) in Assay Medium. A 50 μl aliquot of cells transferred to each well in the assay plate.

4) Stocks of the test compounds and reference compounds diluted to a concentration of 0.2 μM in 0% Assay Buffer for the 0% HSA CRE luciferase assay and 1% Assay Buffer for the 1% HSA CRE luciferase assay. Compounds diluted 10-fold to give the following concentrations: 2×10⁻⁷ M, 2×10⁻⁸ M; 2×10⁻⁹ M, 2×10⁻¹⁰ M, 2×10⁻¹¹ M, 2×10⁻¹² M, 2×10⁻¹³ M, and 2×10⁻¹⁴ M.

5) A 50 μl aliquot of compound or blank transferred from the dilution plate to the assay plate. Compounds tested at the following final concentrations: 1×10⁻⁷ M, 1×10⁻⁸ M; 1×10⁻⁹ M, 1×10⁻¹⁰ M, 1×10⁻¹¹ M, 1×10⁻¹² M, 1×10⁻¹³ M, and 1×10⁻¹⁴ M.

6) The assay plate was incubated for 3 h in a 5% CO₂ incubator at 37° C.

7) The assay plate removed from the incubator and allowed to stand at room temperature for 15 min.

8) A 100 μl aliquot of steadylite plus reagent added to each well of the assay plate (reagent was light sensitive).

9) Each assay plate covered with aluminum foil to protect it from light and shaken for 30 min at room temperature.

10) Each assay plate read in a Packard TopCount NXT instrument.

### Calculations and Results

The data from the TopCount instrument are transferred to GraphPad Prism software. The software performs a non-linear regression (log(agonist) vs response).

### GLP-1 Receptor Binding

The purpose of this example is to test the receptor binding activity of the GLP-1 receptor agonist, such as the GLP-1 derivatives, in vitro. The receptor binding is a measure of affinity of a GLP-1 receptor agonist, such as a derivative, for the human GLP-1 receptor.

### Principle

The receptor binding of the GLP-1 receptor agonist, such as GLP-1 derivatives, to the human GLP-1 receptor are measured in a competitive binding assay. In this type of assay a labelled ligand (in this case ¹²⁵I-GLP-1) is bound to the receptor. Each GLP-1 receptor agonist, such as derivative, is added in a series of concentrations to isolated membranes containing the human GLP-1 receptor and displacement of the labelled ligand is monitored. The receptor binding is reported as the concentration at which half of the labelled ligand is displaced from the receptor, the IC₅₀ value. In order to test the binding of the GLP-1 receptor agonists, such as derivatives, to albumin, the assay is performed in a very low concentration of serum albumin (max. 0.001% final assay concentration as well as in the presence of a considerably higher concentration of serum albumin (2.0% final assay concentration). An increase of the IC₅₀ value in the presence of serum albumin indicates an affinity to serum albumin and represents a method to predict a protracted pharmacokinetic profile of the test substance in animal models.

### Materials

The following chemicals are used in the assay: Human serum albumin (HSA) (Sigma A1653), DMEM w/o phenol red (Gibco 11880-028), Pen/strep (Invitrogen 15140-122), G418 (Invitrogen 10131-027), 1 M Hepes (Gibco 15630), EDTA (Invitrogen 15575-038), PBS (Invitrogen 14190-094), fetal calf serum (Invitrogen 16140-071), EGTA, MgCl₂ (Merck 1.05832.1000), Tween 20 (Amresco 0850C335), SPA particles (wheat germ agglutinin (WGA) SPA beads, Perkin Elmer RPNQ0001), [¹²⁵I]-GLP-1]-(7-36)NH₂ (produced in-house), OptiPlate™-96 (Packard 6005290). Buffer 1 consists of 20 mM Na-HEPES plus 10 mM EDTA and pH was adjusted to 7.4. Buffer 2 consists of 20 mM Na-HEPES plus 0.1 mM EDTA and pH adjusted to 7.4. Assay buffer consists of 50 mM HEPES supplemented with 5 mM EGTA, 5 mM MgCl₂, 0.005% Tween 20 and pH was adjusted to 7.4. An 8% albumin stock consists of HSA dissolved at 8% (w/v) in assay buffer. An 0.02% albumin stock consists of HSA dissolved at 0.02% (w/v) in assay buffer.

### Cell Culture and Membrane Preparation

The cells used in this assay (clone FCW467-12A) are BHK cells with BHKTS13 as a parent cell line. The cells express the human GLP-1 receptor. The cells are grown at 5% CO₂ in DMEM, 10% fetal calf serum, 1% Pen/Strep (Penicillin/Streptomycin) and 1.0 mg/ml of the selection marker G418. To make a membrane preparation the cells are grown to approximately 80% confluence. The cells are washed twice in phosphate-buffered saline and harvested. The cells are pelleted using a brief centrifugation and the cell pellet was kept on ice. The cell pellet is homogenised with ULTRA-THURRAX™ dispersing instrument for 20-30 seconds in a suitable amount of buffer 1 (e.g., 10 ml). The homogenate is centrifuged for 15 minutes. The pellet is re-suspended (homogenised) in 10 ml buffer 2 and centrifuged. This step is repeated once more. The resulting pellet is re-suspended in buffer 2 and the protein concentration is determined. The membranes are aliquoted and stored at minus 80° C.

### Procedure

1. For the receptor binding assay in the presence of low HSA (0.005%) 50 μl of the assay buffer added to each well of an assay plate. Assay continued with step 3.

2. For the receptor binding assay in the presence of high HSA (2%) 50 μl of the 8% albumin stock added to each well of an assay plate. Assay continued with step 3.

3. Test compounds serially diluted to give the following concentrations: 8×10⁻⁷ M, 8×10⁻⁸ M, 8×10⁻⁹ M, 8×10⁻¹⁰ M, 8×10⁻¹¹ M, 8×10⁻¹² M and 8×10⁻¹³ M. Twenty-five μl added to appropriate wells in the assay plate.

4. Cell membrane aliquots thawed and diluted to their working concentration. Fifty μl added to each well in the assay plate.

5. WGA SPA beads suspended in assay buffer at 20 mg/ml. The suspension diluted to 10 mg/ml in assay buffer just prior to addition to the assay plate. Fifty μl added to each well in the assay plate.

6. The incubation started by adding 25 μl of 480 pM solution of [¹²⁵I]-GLP-1]-(7-36)NH₂ to each well of the assay plate. A 25 μl aliquot reserved for measuring total counts/well.

7. The assay plate incubated for 2 h at 30° C.

8. The assay plate centrifuged for 10 min.

9. The assay plate read in a Packard TopCount NXT instrument.

### Calculations

The data from the TopCount instrument are transferred to GraphPad Prism software. The software averages the values for the replicates and performs a non-linear regression. IC₅₀ values are calculated by the software and reported in nM.

### Pharmacodynamic Study in Db/Db Mice

The purpose of the study is to verify the acute effect of the GLP-1 receptor agonist, such as GLP-1 derivatives, on blood glucose (BG) and body weight (BW) in a diabetic setting.

The GLP-1 receptor agonists, such as GLP-1 derivatives, are tested in a dose-response study in an obese, diabetic mouse model (db/db mice) as described in the following.

Fifty-six db/db mice per compound to be tested (from Taconic, Denmark), fed from birth with the diet NIH31 (NIH 31M Rodent Diet, commercially available from Taconic Farms, Inc., US, see www.taconic.com), are enrolled for the study at the age of approximately 10 weeks. The mice are given free access to standard chow (e.g. Altromin 1324, Brogaarden, Gentofte, Denmark) and tap water and kept at 24° C. After 1-2 weeks of acclimatisation, the basal blood glucose is assessed twice on two consecutive days (i.e. at 9 am). The 8 mice with the lowest blood glucose values are excluded from the experiments. Based on the mean blood glucose values, the remaining 48 mice are selected for further experimentation and allocated to 8 groups (n=6) with matching blood glucose levels and body weight. The mice used in experiments with a duration of 48 hours, and for up to 4 times. After the last experiment the mice were euthanised.

The eight groups may receive treatment as follows: 1: Vehicle, s.c. 2: GLP-1 receptor agonist (e.g. GLP-1 derivative), 0.3 nmol/kg, s.c. 3: GLP-1 receptor agonist (e.g. GLP-1 derivative), 1.0 nmol/kg, s.c. 4: GLP-1 receptor agonist (e.g. GLP-1 derivative), 3.0 nmol/kg, s.c. 5: GLP-1 receptor agonist (e.g. GLP-1 derivative), 10 nmol/kg, s.c. 6: GLP-1 receptor agonist (e.g. GLP-1 derivative), 30 nmol/kg, s.c. 7: GLP-1 receptor agonist (e.g. GLP-1 derivative), 100 nmol/kg, s.c. 8: GLP-1 receptor agonist (e.g. GLP-1 derivative), 300 nmol/kg, s.c. The number of groups of mice and specific doses must be adapted as appropriate.

Vehicle: 50 mM sodium phosphate, 145 mM sodium chloride, 0.05% tween 80, pH 7.4. The GLP-1 receptor agonist (e.g. GLP-1 derivative) is dissolved in the vehicle, to concentrations of 0.05, 0.17, 0.5, 1.7, 5.0, 17.0, and 50 nmol/ml. Animals were dosed s.c. with a dose-volume of 6 ml/kg (i.e. 300 μl per 50 g mouse).

On the day of dosing, blood glucose is assessed at time−½ h (8.30 am), where after the mice were weighed. The GLP-1 receptor agonist (e.g. GLP-1 derivative) was dosed at approximately 9 am (time 0). On the day of dosing, blood glucose was assessed at times 1, 2, 4 and 8 h (10 am, 11 am, 1 pm and 5 pm).

On the following days, the blood glucose was assessed at time 24, and 48 h (other time periods, including 72 and 96 hours may be included) after dosing (i.e. at 9 am on day 2, and 3 (and perhaps days 4 and 5). On each day, the mice were weighed following blood glucose sampling.

The mice are weighed individually on a digital weight.

Samples for the measurement of blood glucose are obtained from the tail tip capillary of conscious mice. Blood, 10 μl, is collected into heparinised capillaries and transferred to 500 μl glucose buffer (EKF system solution, Eppendorf, Germany). The glucose concentration is measured using the glucose oxidase method (glucose analyser Biosen 5040, EKF Diagnostic, GmbH, Barleben, Germany). The samples are kept at room temperature for up to 1 h until analysis. If analysis has to be postponed, samples are kept at 4° C. for a maximum of 24 h.

ED₅₀ is the dose giving rise to half-maximal effect in nmol/kg. This value is calculated on the basis of the ability of the GLP-1 receptor agonists (e.g. derivatives) to lower body weight as well as the ability to lower blood glucose, as explained below.

ED₅₀ for body weight is calculated as the dose giving rise to half-maximum effect on delta BW 24 hours following the subcutaneous administration of the GLP-1 receptor agonist (e.g. derivative). For example, if the maximum decrease in body weight after 24 hours is 2.0 g, then ED₅₀ bodyweight would be that dose in nmol/kg which gives rise to a decrease in body weight after 24 hours of 1.0 g. This dose (ED₅₀ body weight) may be read from the dose-response curve.

ED₅₀ for blood glucose is calculated as the dose giving rise to half-maximum effect on AUC delta BG 24 hours and/or 48 hours following the subcutaneous administration of the GLP-1 receptor agonist (e.g. analogue).

The ED₅₀ value may only be calculated if a proper sigmoidal dose-response relationship exists with a clear definition of the maximum response. Thus, if this would not be the case the GLP-1 receptor agonist (e.g. derivative) in question is re-tested in a different range of doses until the sigmoidal dose-response relationship is obtained.

### Pharmacokinetic Study in Minipigs

The purpose of this study is to determine the protraction in vivo of the GLP-1 receptor agonist, such as GLP-1 derivatives, after i.v. administration to minipigs, i.e. the prolongation of their time in the body and thereby their time of action. This is done in a pharmacokinetic (PK) study, where the terminal half-life of the GLP-1 receptor agonist (e.g. derivative) in question is determined. By terminal half-life is generally meant the period of time it takes to halve a certain plasma concentration, measured after the initial distribution phase.

Study A:

Male Göttingen minipigs are obtained from Ellegaard Göttingen Minipigs (Dalmose, Denmark) approximately 7-14 months of age and weighing approximately 16-35 kg are used in the studies. The minipigs are housed either individually (pigs with permanent catheters) or in a group and fed restrictedly once or twice daily with SDS minipig diet (Special Diets Services, Essex, UK).

After at least 2 weeks of acclimatisation two permanent central venous catheters are implanted in vena cava caudalis or cranialis in each animal. The animals are allowed 1 week recovery after the surgery, and are then used for repeated pharmacokinetic studies with a suitable wash-out period between successive GLP-1 receptor agonist (e.g. GLP-1 derivative) dosings.

The GLP-1 receptor agonist, such as GLP-1 derivatives, are dissolved in 50 mM sodium phosphate, 145 mM sodium chloride, 0.05% tween 80, pH 7.4 to a concentration of usually from 20-60 nmol/ml. Intravenous injections (the volume corresponding to usually 1-2 nmol/kg, for example 0.033 ml/kg) of the compounds are given through one catheter or through the venflon, and blood is sampled at predefined time points for up till 13 days post dosing (preferably through the other catheter or by venipuncture). Blood samples (for example 0.8 ml) were collected in EDTA buffer (8 mM) and then centrifuged at 4° C. and 1942 G for 10 minutes.

Study B:

Male Göttingen minipigs are obtained from Ellegaard Göttingen Minipigs (Dalmose, Denmark) approximately 5 months of age and weighing from approximately 9 kg are used in the studies. The minipigs are housed in pens with straw as bedding, six together in each pen and fed restrictedly once or twice daily with Altromin 9023 minipig diet (Chr. Petersen A/S, DK-4100 Ringsted). The pigs are used for repeated pharmacokinetic studies with a suitable wash-out period between successive GLP-1 receptor agonist (e.g. GLP-1 derivative) dosings. An acclimatisation period of 1 week is allowed during which time the minipigs are trained to be fixated on the backs for blood sampling and in slings for i.v. dosing. All handling, dosing and blood sampling of the animals will be performed by trained and skilled staff.

The animals are fasted for approximately 18 h before dosing and from 0 to 4 h after dosing, but have ad libitum access to water during the whole period.

The GLP-1 receptor agonist, such as GLP-1 derivatives, are dissolved in 50 mM sodium phosphate, 145 mM sodium chloride, 0.05% tween 80, pH 7.4 to a concentration of usually from 20-60 nmol/ml. Intravenous injections (the volume corresponding to usually 2 nmol/kg, for example 0.1 ml/kg) of the compounds are given as intravenous injections via a Venflon inserted in an ear vein, while they are placed unanaesthetized in a sling. The dose volume was 0.1 ml/kg, and blood is sampled at predefined time points for up till 17 days post dosing (samples was taken with syringe from a jugular vein). Blood samples (for example 0.8 ml) are collected in EDTA buffer (8 mM) and then centrifuged at 4° C. and 2000 G for 10 minutes.

### Sampling and Analysis (Study A and B):

Plasma is pippetted into Micronic tubes on dry ice, and kept at −20° C. until analyzed for plasma concentration of the respective GLP-1 compound using ELISA or a similar antibody based assay such as LOCI, or LC-MS. Individual plasma concentration-time profiles are analyzed by a non-compartmental model in WinNonlin v. 5.0 or Phoenix v. 6.2 (Pharsight Inc., Mountain View, Calif., USA), or other relevant software for PK analysis, and the resulting terminal half-lives (harmonic mean) determined.

### Pharmacodynamic Study in LYD Pig

The purpose of this experiment is to investigate the effect of GLP-1 receptor agonist, such as GLP-1 derivatives, on food intake in pigs. This is done in a pharmacodynamic (PD) study as described below, in which food intake is measured from 1 to 4 days after administration of a single dose of the GLP-1 receptor agonist (e.g. GLP-1 derivative), as compared to a vehicle-treated control group.

Female Landrace Yorkshire Duroc (LYD) pigs, approximately 3 months of age, weighing approximately 30-35 kg are used (n=3-4 per group). The animals are housed in a group for approximately 1 week during acclimatisation to the animal facilities. During the experimental period the animals are placed in individual pens at least 2 days before dosing and during the entire experiment for measurement of individual food intake. The animals are fed ad libitum with pig fodder (Svinefoder, Danish Top) at all times both during the acclimatisation and the experimental period. Food intake is monitored on line by logging the weight of fodder every 15 minutes. The system used is Mpigwin (Ellegaard Systems, Faaborg, Denmark).

The GLP-1 receptor agonist, such as GLP-1 derivatives, are dissolved in a phosphate buffer (50 mM phosphate, 0.05% tween 80, pH 8; or 50 mM phosphate, 145 mM sodium chloride, 0.05% tween 80, pH 7.4) at concentrations of 12, 40, 120, 400 or 1200 nmol/ml corresponding to doses of 0.3, 1, 3, 10 or 30 nmol/kg. The phosphate buffer serves as vehicle. Animals are dosed with a single subcutaneous dose of the GLP-1 receptor agonist (e.g. GLP-1 derivative) or vehicle (usual dose volume 0.025 ml/kg) on the morning of day 1, and food intake is measured for 1-4 days after dosing. On the last day of each study, 1-4 days after dosing, a blood sample for measurement of plasma exposure of the GLP-1 receptor agonist (e.g. GLP-1 derivative) is taken from the heart in anaesthetised animals. The animals are thereafter euthanised with an intra-cardial overdose of pentobarbitone. Plasma content of the GLP-1 receptor agonist (e.g. GLP-1 derivatives) is analysed using ELISA or a similar antibody based assay, or LC-MS.

Food intake is calculated as mean±SEM 24 h food intake in 24 h intervals (0-24 h, 24-48 h, 48-72 h, and 72-96 h).

Statistical comparisons of the food intake in the 24 hour intervals in the vehicle vs. GLP-1 receptor agonist (e.g. GLP-1 derivative) group are done using two-way-ANOVA repeated measures, followed by Bonferroni post-test.

### Pharmacokinetic Study in Rat

The purpose of this Example is to investigate terminal half-life in vivo in rats.

In vivo pharmacokinetic studies in rats are performed with the GLP-1 receptor agonists (e.g. GLP-1 derivatives) of the interest, as described in the following.

Male Sprague Dawley rats of same age with a body weight of approximately 400 g are obtained from Taconic (Denmark) and assigned to the treatments by simple randomisation on body weight, approximately 4 rats per group.

The GLP-1 receptor agonist (e.g. GLP-1 derivatives) (approximately 6 nmol/ml) are dissolved in 50 mM sodium phosphate, 145 mM sodium chloride, 0.05% tween 80, pH 7.4. Intravenous injections (1.0 ml/kg) of the compounds are given through a catheter implanted in the right jugular vein, or they are dosed IV via the lateral tail vein. Blood is sampled from vena sublingualis for 5 days post dosing. Blood samples (200 μl) were collected in EDTA buffer (8 mM) and then centrifuged at 4° C. and 10000 G for 5 minutes. Plasma samples were kept at −20° C. until analyzed for plasma concentration of the respective GLP-1 compound.

The plasma concentrations of the GLP-1 compounds are determined using ELISA or a similar antibody based assay such as LOCI, or LC-MS. LOCI refers to Luminescence Oxygen Channelling Immunoassay, which is generally described for the determination of insulin by Poulsen and Jensen in Journal of Biomolecular Screening 2007, vol. 12, p. 240-247. The donor beads are coated with streptavidin, while acceptor beads are conjugated with a monoclonal antibody recognising a mid-/C-terminal epitope of the peptide. Another monoclonal antibody, specific for the N-terminus, is biotinylated. The three reactants are combined with the analyte and formed a two-sited immuno-complex. Illumination of the complex released singlet oxygen atoms from the donor beads, which are channelled into the acceptor beads and triggered chemiluminescence which is measured in an Envision plate reader. The amount of light is proportional to the concentration of the compound.

Individual plasma concentration-time profiles are analysed by a non-compartmental model in WinNonlin v. 5.0 or Phoenix v. 6.2 (Pharsight Inc., Mountain View, Calif., USA) or other relevant software for PK analysis, and the resulting terminal half-lives (harmonic mean) determined.

### Tables

TABLE 1
Subject characteristics at baseline were comparable between groups

| | TREATMENT GROUPS | | |
|---|---|---|---|
| | 0.6 mg | 1.2 mg | 1.8 mg |
| | group | group | group |
| | n = 15 | n = 14 | n = 16 |
| Sex (n %) | | | |
| Female | 6 (40.0) | 3 (21.4) | 7 (43.8) |
| Male | 9 (60.0) | 11 (78.6) | 9 (56.3) |
| Age (years) | 38.9 ± 11.3 | 34.6 ± 12.5 | 30.4 ± 9.0 |
| Height (m) | 1.76 ± 0.10 | 1.77 ± 0.07 | 1.74 ± 0.06 |
| Body weight (kg) | 75.4 ± 14.0 | 72.5 ± 8.9 | 74.2 ± 11.2 |
| Duration of diabetes (years) | 17.7 ± 9.2 | 18.5 ± 10.3 | 13.9 ± 8.9 |
| HbA_{1c} (%) | 7.84 ± 0.88 | 7.49 ± 0.71 | 7.56 ± 0.68 |
| Daily actual insulin dose (U) | 48.7 ± 15.1 | 42.5 ± 11.2 | 44.9 ± 13.4 |

Data are presented as mean ± SD unless otherwise stated.
Abbreviations:
n = number of subjects;
U = unit

TABLE 2
Hypoglycaemic episodes during treatment

| | HYPOGLYCEMIC EPISODES | | |
|---|---|---|---|
| | N % E | | |
| | 0.6 mg | 1.2 mg | 1.8 mg |
| Classification of | liraglutide/ | liraglutide/ | liraglutide/ |
| hypoglycemic events | placebo | placebo | placebo |
| Total ADA classified | 14 (93.3) 289/ | 13 (92.9) 263/ | 14 (100) 316/ |
| | 14 (100) 313 | 13 (100) 326 | 15 (100) 295 |
| Severe | 0 (0) 0/ | 0 (0) 0/ | 0 (0) 0/ |
| | 0 (0) 0 | 0 (0) 0 | 0 (0) 0 |
| Documented | 13 (86.7) 125/ | 12 (85.7) 161/ | 14 (100) 152/ |
| symptomatic | 13 (92.9) 146 | 10 (76.9) 193 | 14 (93.3) 154 |
| Asymptomatic | 12 (80.0) 114/ | 10 (71.4) 73/ | 13 (92.9) 134/ |
| | 13 (92.9) 113 | 11 (84.6) 84 | 14 (93.3) 109 |
| Probable symptomatic | 1 (6.7) 3/ | 1 (7.1) 1/ | 1 (7.1) 4/ |
| | 1 (7.1) 2 | 2 (15.4) 6 | 1 (6.7) 2 |
| Relative | 5 (33.3) 47/ | 8 (57.1) 28/ | 8 (57.1) 26/ |
| | 6 (42.9) 52 | 6 (46.2) 43 | 6 (40.0) 30 |

Abbreviations:
E = number of events;
N = number of subjects;
% = percentage of subjects
Severe: an event requiring assistance from a third party.

TABLE 3
Adverse events during the trial

| | ADVERSE EVENTS | | |
|---|---|---|---|
| | N % E | | |
| | 0.6 mg | 1.2 mg | 1.8 mg |
| | liraglutide/ | liraglutide/ | liraglutide/ |
| | placebo | placebo | placebo |
| Total number of events | 12 (80.0) 27/ | 14 (100) 45/ | 13 (92.9) 54/ |
| | 9 (64.3) 17 | 12 (85.7) 24 | 10 (66.7) 18 |
| Serious AEs | 0/0 | 0/0 | 0/0 |
| AEs leading to withdrawal | 1 (6.7) 1/0 | 1 (7.1) 1/0 | 1 (7.1) 1/0 |
| | GASTROINTESTINAL | | |
| | ADVERSE EVENTS | | |
| | N % E | | |
| Total number of GI AEs | 10 (66.7) 12/ | 12 (85.7) 24/ | 13 (92.9) 31/ |
| | 2 (14.3) 2 | 1 (7.7) 2 | 3 (20.0) 5 |
| Nausea | 8 (53.3) 8/ | 11 (78.6)14/ | 11 (78.6)13/ |
| | 1 (7.1) 1 | 1 (7.7)1 | 2 (13.3)2 |
| Diarrhea | 0/ | 1 (7.1) 1/ | 5 (35.7) 5/ |
| | 1 (7.1) 1 | 1 (7.7) 1 | 1 (6.7) 1 |
| Vomiting | 1 (6.7) 1/0 | 1 (7.1) 1/0 | 3 (21.4) 4/0 |
| Regurgitation | 1 (6.7) 1/0 | 2 (14.3) 2/0 | 1 (7.1) 1/0 |
| Upper abdominal pain | 0/0 | 0/0 | 2 (14.3) 2/ |
| | | | 1 (6.7) 1 |
| Abdominal pain | 0/0 | 1 (7.1) 1/0 | 0/ |
| | | | 1 (6.7) 1 |
| Dyspepsia | 0/0 | 1 (7.1) 1/0 | 1 (7.1) 1/0 |
| Flatulence | 1 (6.7) 1/0 | 1 (7.1) 1/0 | 0/0 |
| Abdominal distention | 1 (6.7) 1/0 | 3 (21.4) 3/0 | 5 (35.7) 5/0 |

Abbreviations:
AE = adverse event;
E = number of events;
GI = gastrointestinal;
N = number of subjects
% = percentage of subjects

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for reducing the amount of exogenous glucose needed for recovering from hypoglycaemia or a hypoglycaemic episode, comprising administering a GLP-1 receptor agonist to a patient in need thereof
iv).

2.-5. (canceled)

6. The method according to claim 1, further comprising administering one or more additional anti-diabetic drugs.

7.-8. (canceled)

9. The method according to claim 1, wherein the amount of exogenous glucose needed is reduced by at least 5%.

10. The method according to claim 1, wherein the GLP-1 receptor agonist is a GLP-1 fragment, derivative, or analogue, and wherein the GLP-1 receptor agonist comprises no more than 5 amino acid residues that have been substituted, inserted or deleted as compared to GLP-1(7-37) (SEQ ID NO:1).

11. (canceled)

12. The method according to claim 1, wherein the GLP-1 receptor agonist is selected from the group consisting of: GLP-1(7-37) (SEQ ID NO:1); GLP-1(7-36) amide; Exenatide; Exenatide LAR; Liraglutide; Semaglutide; Taspoglutide; Albiglutide; Lixisenatide; and Dulaglutide.

13. The method according to claim 1, wherein the GLP-1 receptor agonist is Liraglutide.

14. The method according to claim 1, wherein the GLP-1 receptor agonist is administered with a pharmaceutically acceptable carrier, vehicle, diluent, and/or excipient.

15. (canceled)

16. The method according to claim 1, wherein the GLP-1 receptor agonist is administered in the range of 0.5-2.0 mg/day.

17. The method according to claim 16, wherein the GLP-1 receptor agonist is administered in the range of 0.6-1.8 mg/day.

18. The method according to claim 17, wherein the GLP-1 receptor is Liraglutide.

19. The method according to claim 17, wherein the GLP-1 receptor agonist is Liraglutide and is administered at 0.6 mg/day.

20. The method according to claim 17, wherein the GLP-1 receptor agonist is Liraglutide and is administered at 1.2 mg/day.

21. The method according to claim 17, wherein the GLP-1 receptor agonist is Liraglutide and is administered at 1.8 mg/day.

22. The method according to claim 6, wherein the anti-diabetic drug is insulin, wherein the GLP-1 receptor agonist is Liraglutide, and wherein the insulin is administered concurrently with the Liraglutide.

23. The method according to claim 6, wherein the anti-diabetic drug is insulin, wherein the GLP-1 receptor agonist is Liraglutide, and wherein the insulin is administered consecutively with the Liraglutide.

24. The method according to claim 9, wherein the amount of exogenous glucose needed is reduced by at least 10%.

25. The method according to claim 9, wherein the amount of exogenous glucose needed is reduced by at least 15%.

26. The method according to claim 10, wherein the GLP-1 receptor agonist comprises no more than 3 amino acid residues that have been substituted, inserted or deleted as compared to GLP-1(7-37) (SEQ ID NO:1).
